SEQUENCE LISTING (47 sequences)

SEQ ID NO: 1 - DNA, 1446 bp, Mus musculus
ggtcccatgccactagtgggagctgtgttacctgttgcagccccacccaaagcccctgctataggtcaagcaggaatcaccctgccatccccagcctggggcctggagtaccagatccaggaaactagcatcccttagctatagagatagccacacatcagcccattcctcagatgtgtatctggggctcagacatcatctcccgatctccgacaagggcaggatttccttaccgtctgatggggtctctgctggtatcctcagcccctagtctccagccttcaggcatgccgggcctattgagatgggagaacttggtaccggggtcctgtgcccaggaccctagcagtccccagctcaggtacaccccaaagcccagcagcagtgtcgggatcatggtgaggggctcctgtgctgatgctcagccttacaagtgactctcaaatttgctggtgatgtggtcttcaagcgaaatgtcagaaagaaaagaaaacacgaggacaacaaagggaggaagtggcctggtccggcccacccggcaagtctcatccgcccccgcccccgccccttccagcctggcccccttggaggcctccaaccactcaggtcaattcctgtgtcctgagggcacttgaatcagggacacgggatttggtagacacataaaggtggccccattaaacttatttctccaggactctgtcgtgggcctggaggagatctggtgccacccatactgttggccaggaagtggggaacgggcacatcacacctgctcggcaccttgggctatgggaactagcaggtgggtgggaactcagagaaggaaagggactatgctagaatcacacagcgggcagcccagtctagggcatggggagcagctttgggtgtttctggcctcagccttccaacaggtttggctagagctccaggctcaagagcatccaggatacagtggggaactggataacagggcagcctgcaggttggccattcattggattggccctgaccccggcccagcctgggacactgaggcatcatcagtcaaggcacttttcttctgcatataagagccagggcacgagaccaccagggctttccaaggatgaatgaggtgtaatgatagattaggatatgtccagcctccaacacgctctccctcccccagggccaacaagagtcagcagggcagaatagagccagtaggggcccgggccctgctcgctggtatccccgtgaggcatgccttctctctggcccgccctccctgcccccaccctggcccgggctggctgggctccagccagcagccacagcatcaatatttcatctgcgtcaataagaggcagtagcagcagagacagcggctgcggcagcactccagtccatagcttgcagcagccaggttccatccttgcaaacaccatggacgtcttcaag

SEQ ID NO: 2 - DNA, 1120 bp, Mus musculus
tgagccttcttattcagaccaccaaaattactttatttttccacatgaaagtattatgtggcttcctgtctgcaaagaggaagacatccatgaacactaatgacactgggtttgggctatgtccggagcagaggaatgaggccatgtagactaaatatgtgcatgtggaggaggctgaaaacacatcagagctagcgctgcaggaaatgcttctgcattgcatacccagagtttccttgctcatctgggagtctgtgtttttcctagatgtgtgcacttgtgtgagattctctgggtgtgagtcaaagtgttatctgaatgtgtaatgtgtgctcaatatgctcatgtgtgttaccctgagcttctgtgtctacatatatacctggatgcctgtgtgttctgtgatgtacatatatattctgtctttccttccttttctatttgtgttattccatgtgttctttcagattctcaccaccaagggcaaggatatgttaactacccaagtgtccacctccgcctgtctggtgatgtttacgccacccccgtgctcttttctttgcccgacagagttgttataggagatgtctccccgggaacactgcaggaaggagaatttctacatttatgttcccctctgagtgtgcttctatccccaaaatgccttcaaaggtgaaaatcaacactggaaacccaagtatctgggaagggcaagagtgtgtaagtgcaagttagcctaaggaataggaagaggttggtaaacagggtaggatcgtgggagggagtttcgttactacaggtccggaccctcaggacaagaaccccacccccactccccaaattgcgcatcccccgcccccatcagagggggaggggaagaggttgcggcgcggcgcatgcgcactgtcggattcagcaccgcggtcagagccttcgcctccgctgccggcgcgcaccaccacctccccagcaccaaaggctgactgacgtcactcactagccctccccaaactccccttcctcgccgccttggtcgcgtccatgctgccgtgagtccagtcggaccgcaccacgagaggtgcaagatagggaggtgcgggcgcgaccatacgctctgcgg

SEQ ID NO: 3 - DNA, 2807 bp, Homo sapiens
actcagtcctttttgtgctgtctcctcctctttcccagagttcctctctctcttctcccactaggcagggatgagcaagaggaatggctcacccttgagagctggggtccatagcccaggtcagttctccagctctcccacttaccagccaagacaggaggtgaggattgagatgggatgaacccagcaggcggccatgggttaaaggtcgccatgaatgtaatgtgcccagcacagtgcctgctaaaaggcaacactcccttcctggtctgaagaccaaacaagcagactgtactcaggaaagccagaagaaccttccagctgtctggaccagaaggtgccagcccaggggctgaagaagacgtaatgcccagagcaaaaagcgcctgcagccccctgaagggctgggtgctctggaatagatgagggggcgaaatggggctggggaccagggacggacagggtgggtccagcacctgcctcgcttccgaagggctgctccaacactgaaaaacacccaaccagcttcctttcagaaagactggaatattccaaaacttctcactggaggctccggaggaggtgggctccagctgaaaaggaaatgtggaggcgtgggcgctcccggcctgcatcctgcacctcttacactttggttttcccacagactcctgaagaataggtcagaagaaagggttaaagccttaaaaggggaacaaccattgcggggctcagggaggaggataatgttctttgggctgccgcaccctgatccccggggtcccgaaccctcccgtccctggccaggcctgccagccacagggtgagggcccccttccgccgcaacctgccactctcacaccaatgcgggaccgccttctcttccttccccaccccccaccccaccctgccgtcctttctcccccaatctccgcctctgattggctgagcccccggctccccgctccccctctcctccatccccggtgaaaactgcgggctccgagctgggtgcagcaaccggaggcggcggcgcgtctggaggaggctgcagcagcggaagaccccagtccaggtgggaactggagccggtgggacctggggctcggggaccgccgtcaggcgcccatgcaagacttcccaacactaggcttcgggccacggtccgagggcgcccagggaagaagggcgcagagcttagggaggggcctgctttccaggcaggggcgggagggggatgcttctgcagggcaggggccgcgtggcaccctgatgtctttcggggaaggcgctcccgggctttcgcccgctgggggactggtgtctggggctggggcgctggagaacagggaggaagggcaccaaggacagcctgtgggtctacattccacccagacgtccccaaacccagctcgcagaggcggggaggaggacggatgaaactgcggggagaggatggaggatggcgagctagagggaatctgccgggtgacctcgcggcgggctgggtgcggggcaccggaggagaaggaagccgcagtgccgcaggcggggactgggtggaaggcgggcggacgggggaggggagagctggaaaaggatgagagagggggaagggggactcatttgggaaaggagaggattggaatacggaaatggattaaggatgaggcccgccgggggcttgagagggaggaagagcagaccttctctgggtctggagccgcctgaggacacagaccagaggaaatgaatacagactgcacctccccagccgctctccacccctcccctggctcttctaccctctccagccccagacccatttcttccctttcttgctctggccattgctcccccttcccctcctagatcccaagcccgcacaacatctcaaacaagagtcctcgattcaaaagccagatgccgaccccccttcctcctggatctggctcagggcagcagctccaccccgggacagagagagcattgattgtagctgcagccgccgcgggatcctagcctcacccgtcaaggggctgagcgccagggaccctgaactcgtctagtggtgcgccctgcgcacccgggcgcactcaaccgaggcaatgccctgcgcgctctcgcgggtgcacgccccttctgtggcctctcctgggcgagcactgctctgcagataggctagactaccggctccgcgtcgcctcgccaagggttggttcagccaaggctgcaaaaaacaaaaaaagaccaggcagacagcctatccagggtggctattgaaactgggctggaaaactgcagtcccaggaactccagagagctggacattgggaagcatccttggctcacatacaatcggagatcactatgtctttctctcctccaggaacacgattagcttgtgtcctatccagataggaatagatgctccctatctgggagcatccttagctatggtgaatggtatctagccatccactggggatggcgagtgacttagggatttgtgtctcacgtatatgaagcagtcatcgccagatgttggttgtttttcttaacccccatcataacccggtgggtatgtaagattcagagagattcattcattcattcacaataaatatctttggagtgtatgctatatgccagtaatctgcaaacggaaacggttttgagcattggggattttcttctgaacaggaaatgggaagtccctaaatggggagtctttgtttaacagatacagagttttactttgaaagacaaaaagagttccggagatgggctgcatagcaacgtgaatg

SEQ ID NO: 4 - DNA, 2500 bp, Homo sapiens
cataggcagcttcaatctgatggctgtggccccttggcctcaacagaatacatcttggagccccctttttaccccaaacccccattcctccttgctgtcagctgcttgtgagccttctcacatccagagaatgtatcagcattgtgcagactgaaaagacccagaggaacaaggctccaatggcaaaattccaagtagaatgacaaataaatggggagccatctgagagcaagggagtcctgcccaacacccgccccatgcctttctcagggacctcagaccagccactcacctccatcctcccagcaccacctgcaaccagccccttgccctctgcaaactggagcacgactggatctttagatgggggaaaaatgcttcatcatgttctgctgcttcatgcaaaaccagaaactccctccccctcttccctcctcccagcgcactctccttccagtaaaaagtggttaaagggacagcgccatcaatttcccagctctgagggtctgcttagaactagggggctggaaggagacagagggcaaagagaaaggaactggcagaggtctttcctgggggatatgtctgttctgtcctggggatcctggagcaggaaaacccgcgtaaagtaggggtgtagtgggtgttgagataactgcctgggggaggttcagagtggaagtacgagtctacaaactctcaagggcgtctcagggctcccagcatccccaggggtcctttcgcaggggtccctaagcaggaggggaacagcccagaaaacacggaactggacccccgacaggaagtccagggaggggtccctggctcactatgtgaccctgctggatcacttgcctcccctctcgggtcccctcagcacagtgtccctcccttccttcccctaaagtaaaagcagagggttaatctctttccccgccccacgcccaacaaagagcaggccctgtccccggtgctgaagcgccagccgcagcaccacccccactcccacagcataaaacatgagccaaaaccaataaagagccaaatgtcacagccgttgcagggccccctaaatcctggggaccccttcttctacctgacatcctattggggtgagggactttggtactcagaaagcatctcatcacttccctgtaagagagaagggatgccgactcaggcgcctgcttgtctgttacaggagtgggggaagagaggacaagttgaggctgagaagatggggagggggagggagaaaagaggacttcctagtgttgacagaacggcaagatgtgggttccccatccccagttcagccagagacccctcaaagtggaacttcctggggcagtcgggggtcaggagttggagcttgtctctggggcaagaccccttcgttgtacagatggaaaaacaagggtgggaggacacagcttgtccaaggtcattcgaccagcaaactgcctagctgaccccagtgtgcagaagctggctcgggtgacacccatcatttccccccaccccacacaggggccagctctctcaacttcatgcccaagccctcctacggtacccccactgtaggttctctgcccctcaaactcagcccagctttctcctgcctgttcaggggaccttctgcccgcttcgctgagggtccgtcccctttactggggctggcagcagggtctcccatctcctctctcgggggccactgcagactttttagagaacgccttgcctccccccaaccccacccatccggggttccctctctccatcctctgcagtgtctcccatacccccattcagggtagccttgctattctccccaactccaggtcccccttcatctattccggggctggccgcggagtttcctgagcgctctccaagtgggtcctctagatgttaggagaacactgtacctcccccggtcaggggtctcctgtctccgttctatggagcgtccatgctcccattcaggactgccttgctccctcctctgttccggggctggctgcacagtctctgcaccccctatcctgaaagcctctcttaactatttggaaagcctcgtgtcctgtctcatacagggatcccctcatcctaatgactgcaatcttccattgctccatcccgagggcatcctgcccctattcccatcaggtttctccttgtcctctccctgtttcaagtcccctttcttattccgaacacactcgcaggctcttccgacgcgcacccgggggtcctcactggcccactccgggagtcctctgcccgcttccccgacctcgagggtctcctctgacgcagcgtcgattccccttccctcctcggtcccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgcc

SEQ ID NO: 5 - DNA, 2500 bp, Homo sapiens
gccttggctgtcctggaactcactcaggctgagtgaggctgacttcagattcacagagatctgcctgcctgcctctgcctcctgaatgctgggattaaaggcgtgcgctaccactgcacagcaaaaagaatcattctcagctctctctgggccatacgttttgctagagagctgattagaattcatccatctatccactcacaatgacaaactgggaagcagcatgcgggcaaggaccacagaaccccaggaggggacaaggctcagggtgaagggaaggtgaggctgaaggactgaccagggtctcagcattcatgacaaccttacagccacaaaagccacaccttttaccttcacacacaccccactcctatcattcatgtgtgctgtcaactgcttgtcagacttctcacccccaagaagggcatgtgcattctgcagacaactgaagagactcgaaggaacaagaatctaataacaaaaatccaagcagtatgggagataaatggggaagccatgtgggcgtaagggggtagaggtctgcatcccagtcccctccccatggcatctgcagtgcctcccagcctttctgacccctgcaaagagcagcatgactggacctttaaattgggaaaatgcttcatcatgttctgctccatcatgaaaaactagagtctcctccccctcctccctagtgcactctcctggcctgcagccaggggctgggaatgagacacaggacaggaaagggatctcttttagggaatctatcagttctcctcctagggatccctccaaaagagaaaaccacagcaaactggggtgcagtgaggcttgaggtaactgcctgggagaagttctgatctgaagaagtctatactggtttccagagcttgtcagtgggcattggagtggggctctctctgctccgggaagaggtttgcagggagaaagaacttcacagagagccaggcactggacaggacatgcaggggtgggtcacttacatacaaccgtaggtcgtttcgagcccgtcatatgactcatccaatcctcccctgtaccgcacagagggactgcttggaaaagctatggaacctccctactccgttaggcatagatttaacccttcccatccgaggagcggctgctgtccgtggtgctgaagcgatagcggcacgggcggctccgtccactaacaccgcttttgaccggaaaaccaaaccaagaacgagccgtataataaagcaagagctccaagtctaagcccctccgccgtccccgccctttcacctgaagcctcagtagggctcatgatggaggtcggtggactttggtactgaaaaaccactccaccacttcctcggagcatgaaaggggatgcttacggcagtactggttcatctattctggaaaaggaatgagatgccaagataaagcagaaaaatcgggcaaggaagggagaaagacaaagttctcaggtgagaggaactggttactattccgactggcaatatgtgggttctcctccccaaaatcagccagacatttcccaagttcgaacctcctaggggcacatgggagcttggagctgcatcttgtctcttgcacacaagggaaaaccaaacataggagaacacaatttgtacaaggtcattcagctagcgaagcacagaagctaaccccaccctgtggcagaacttggcttcggtgttgaggctcttgctgcctactgagggaccccctgttcttcgtaggcagttttcctttccgggcaagaggagactccactttccagtcgtggccactggaatttttagagagcaccacgttcctctcacccagcgctccctttctccgtctgcagtgttctccttctcagggtagctttgcggtcctttcaaactccacgcccaccccaaccccaaccccgaagccagctgtacagttccttaagcccctttgggtggcccagggccgctgtagtatctggggaacactgcaccgccagctagaaggtccccatttatcatcagtagcatccatcatgcaaccccatacagaatcccttcgtgggtgactgcagtctgcactcctcatctcaaggtcctctctaactatcagggaaccaaccctgtgctgcttctcaagtgggggtgtcctctcatagtaatcactgcagtctcccactgcttcaacccgaaggcgccctgacccatcagttctgcaatcctctccctatttccagtgccctctcttattctgagggtcttattctgactaatagggtcttccgacatgcacctggaggtctgcacttgtccgctccggaagtcctttactccttggtctgacctcgggaggctctactgacgatgcgtcgattccccttcactcctgggtcgtcccccccagccccgcccctctcactgcggagaagccggtcggcccggggccgcgggggaggaggtggagagggtggggccctcctccccagccc

SEQ ID NO: 6 - DNA, 199 bp, mus musculus
ccctgccccgcccctctcactgcggcggagccggtcggccggggggccgcaggggaggaggcggagagggcggggccctcctccccaccctctcactgccaaggggttggacccggccgcggcggctataaaagggccggcgccctggtgctgccgcagtgcctcccgccccgtcccggcctcgcgcacctgctcaggc

SEQ ID NO: 7 - DNA, 1349 bp, homo sapiens
ccaaggtccaggccctcttaccgtcctacggggtccttgccggtgtcctcagcctctgccttccagaccccaggtgtcctggagctctgcagatcagagaggctagtactggaagcctgggcccatgcccccagcagcccccagcccagggggcccccaaggctgaacagcaagctcaggatcatcttggtggtggggcaggctcagctcacactcagccttggcaagtagctccagaaactgctagtgacgttgtcttcaagttaaatctcaggaggaaaagaaaatacgaggacaacaaagagaggaagtggcctgggccggcctacccggtgggtcttgtcctgccccccaactaccctggctggccccacaggggccgccaaccacacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaagggaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacacccaccatggatgt

SEQ ID NO: 8 - DNA, 453 bp, homo sapiens
atgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtc

SEQ ID NO: 9 - DNA, 2954 bp, mus musculus
gagcagatctccagccaagaggcaaaggaatgggggaagctggagggcctccctctggttatccaggcttctgaaggttcaagcaaagaaagggttacaaccttaaaaggagagcgtcccggggtatgggtagaagactgctccaccccgacccccagggtccctaaccgtcttttccctgggcgagtcagcccaatcacaggactgagagtgcctctttagtagcagcaagccacttcggacacccaaatggaacacctccagtcagccctcgccgaccaccccaccccctccatccttttccctcagcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgaggatgcaagccttccccagctacagttttgggaaaggataccagggcgctcctatatgggggcgcgggaactggggaaagaaggtgctcccaggtcgaggtgggagaggaaggcagtgcggggtcacgggctttctccctgctaacggacgctttcgaagagtgggtgccggaggagaaccatgaggaaggacatcaaggacagcctttggtccccaagctcaaatcgctttagtggtgcgaatagagggaggaggtgggtggcaaactggagggagtccccagcgggtgacctcgtggctggctgggtgcggggcaccgcaggtaagaaaaccgcaatgttgcgggaggggactgggtggcaggcgcgggggaggggaaagctagaaaggatgcgagggagcggaggggggagggagcgggagaatctcaactggtagaggaagattaaaatgaggaaatagcatcagggtggggttagccaagccgggcctcagggaaagggcgcaaagtttgtctgggtgtgggcttaggtgggctgggtatgagattcggggcgccgaaaacactgctgcgcctctgccaaatcacgctacccctgtatctagttctgccaggcttctccagccccagccccaattcttttctctagtgttcccccttccctcccctgaatctcaagcccacactccctcctccataacccactgttatcaaatccaagtcatttgccacccaacaaccatcaggaggcggaagcagacgggaggagtttgagatcaacttgggctacatcacgagttccaggctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcgcagactgtgcctctcttgggcaaggacaggctagacagcctgccggtgtgttgagctagggcactgtggggaaggcagagaacctgtgcagggcagcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagaccccatcaaaatcctatttgagtgtacggttcggagaacctcatttatccggtaaatgtcttttactctgctctcagggagctgaggcaggacatcctgagatacattgggagaggagatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctgcctctgcctctctgcctctctgcctctctgcctctctctctgcctctctctgcctctctctgcccctctctgcccctctctgcccctctctgcccctctctgccgccctctgccttctgccctctgccctctggcctctggcctctgccctctgccctctggcctctggcctctgcctctgcctcttgagtgctggaatcaaaggtgtgagctctgtaggtcttaagttccagaagaaagtaatgaagtcacccagcagggaggtgctcagggacagcacagacacacacccaggacataggctcccacttccttggctttctctgagtggcaaaggaccttaggcagtgtcactccctaagagaaggggataaagagaggggctgaggtattcatcatgtgctccgtggatctcaagccctcaaggtaaatggggacccacctgtcctaccagctggctgacctgtagctttccccaccacagaatccaagtcggaactcttggcacc

SEQ ID NO: 10 - DNA, 471 bp, Artificial Sequence
  Laboratory synthesized sequence
atgggagcccgggagatctggatctgggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatgtggccgacttcgagtttgagcagatgtttaccgatgcccttggcattgacgactttgggggggggcgcgctacacagcctgctgaagaagcagcacgaaagagagaggttcgtctaatgaagaacagggaagcagcaagagaatgtcgtagaaagaagaaagaatatgtgaaatgtttagagaacagagtggcagtgcttgaaaaccaaaacaaaacattgattgaggagctaaaagcacttaaggacctttactgccacaagtcagattaa

SEQ ID NO: 11 - protein, 156 aa, Artificial Sequence
  Laboratory synthesized sequence
MGAREIWIWAPPTDVSLGDELHLDGEDVAMAHADALDDFDLDMLGDGDSPGPGFTPHDSAPYGALDVADFEFEQMFTDALGIDDFGGGRATQPAEEAARKREVRLMKNREAARECRRKKKEYVKCLENRVAVLENQNKTLIEELKALKDLYCHKSD

SEQ ID NO: 12 - DNA, 942 bp, mus musculus
atgctcaaagtcacggtgccctcctgtccctcctcgccctgctcctcggtcaccgccagtactgagaacctcgtcccggattactggatcgacggctctaaccgagatcctctgggcgatttcttcgaggtggagtcagagctgggacggggtgctacatccattgtgtacagatgcaaacagaaggggacccagaagccctatgctctcaaagtgttaaagaaaacagtggacaagaagattgtgagaacagaaataggagttctcctgcgtctctcacacccgaacatcataaaactcaaggaaatattcgaaacccccacagaaatcagcctggtccttgagctggtcacaggaggagaactgtttgacaggattgtggagaagggatactacagtgagcgcgatgcggctgacgcggtgaagcagatcctggaggccgttgcttacctgcatgaaaatgggattgtccatcgtgacctcaaaccagagaatcttctttatgcaactccagcccctgatgcacccctcaaaattgctgattttggactttcaaaaattgtggaacatcaagtgctcatgaagacagtgtgtggaaccccggggtactgcgcacctgagattctccgaggctgtgcctacggacctgaggtggacatgtggtctgtaggaataatcacctacatcctactttgtggatttgaaccattctatgacgagcgaggtgatcagttcatgttcaggagaattctgaattgtgaatattactttatctccccctggtgggatgaagtgtctttaaatgccaaggacttggtcaaaaagctcattgttttggatcccaagaaacggctgactacatttcaagccctccaacacccatgggtcacaggtaaagcggccaactttgttcacatggacactgctcagaagaaactttaa

SEQ ID NO: 13 - protein, 313 aa, mus musculus
MLKVTVPSCPSSPCSSVTASTENLVPDYWIDGSNRDPLGDFFEVESELGRGATSIVYRCKQKGTQKPYALKVLKKTVDKKIVRTEIGVLLRLSHPNIIKLKEIFETPTEISLVLELVTGGELFDRIVEKGYYSERDAADAVKQILEAVAYLHENGIVHRDLKPENLLYATPAPDAPLKIADFGLSKIVEHQVLMKTVCGTPGYCAPEILRGCAYGPEVDMWSVGIITYILLCGFEPFYDERGDQFMFRRILNCEYYFISPWWDEVSLNAKDLVKKLIVLDPKKRLTTFQALQHPWVTGKAANFVHMDTAQKKL

SEQ ID NO: 14 - DNA, 1410 bp, mus musculus
atgctcaaagtcacggtgccctcctgtccctcctcgccctgctcctcggtcaccgccagtactgagaacctcgtcccggattactggatcgacggctctaaccgagatcctctgggcgatttcttcgaggtggagtcagagctgggacggggtgctacatccattgtgtacagatgcaaacagaaggggacccagaagccctatgctctcaaagtgttaaagaaaacagtggacaagaagattgtgagaacagaaataggagttctcctgcgtctctcacacccgaacatcataaaactcaaggaaatattcgaaacccccacagaaatcagcctggtccttgagctggtcacaggaggagaactgtttgacaggattgtggagaagggatactacagtgagcgcgatgcggctgacgcggtgaagcagatcctggaggccgttgcttacctgcatgaaaatgggattgtccatcgtgacctcaaaccagagaatcttctttatgcaactccagcccctgatgcacccctcaaaattgctgattttggactttcaaaaattgtggaacatcaagtgctcatgaagacagtgtgtggaaccccggggtactgcgcacctgagattctccgaggctgtgcctacggacctgaggtggacatgtggtctgtaggaataatcacctacatcctactttgtggatttgaaccattctatgacgagcgaggtgatcagttcatgttcaggagaattctgaattgtgaatattactttatctccccctggtgggatgaagtgtctttaaatgccaaggacttggtcaaaaagctcattgttttggatcccaagaaacggctgactacatttcaagccctccaacacccatgggtcacaggtaaagcggccaactttgttcacatggacactgctcagaagaaacttcaagaattcaatgctcggcgcaagcttaaggcagcggtgaaggctgtggtggcctcttctcggctgggaagtgccagcagtagccacaccagcatccaagagaaccacaaggccagctcggatccaccttcaacccaagatgccaaggacagcacagatcttctgggaaagaaaatgcaagaggaggaccaagaggaggaccaagtggaggccgaggcttcagccgatgagatgaggaagctgcagtccgaggaggtggagaaagatgcaggtgtaaaagaggaggagacctccagtatggtgcctcaggatccagaggatgagctggaaacagatgacccagagatgaagagggattcagaggagaagctgaagagtgtggaggaagaaatggaccccatgactgaggaggaagcccctgacgcgggacttggggttccacagcaggatgcgattcagccagagtactaa

SEQ ID NO: 15 - protein, 469 aa, mus musculus
MLKVTVPSCPSSPCSSVTASTENLVPDYWIDGSNRDPLGDFFEVESELGRGATSIVYRCKQKGTQKPYALKVLKKTVDKKIVRTEIGVLLRLSHPNIIKLKEIFETPTEISLVLELVTGGELFDRIVEKGYYSERDAADAVKQILEAVAYLHENGIVHRDLKPENLLYATPAPDAPLKIADFGLSKIVEHQVLMKTVCGTPGYCAPEILRGCAYGPEVDMWSVGIITYILLCGFEPFYDERGDQFMFRRILNCEYYFISPWWDEVSLNAKDLVKKLIVLDPKKRLTTFQALQHPWVTGKAANFVHMDTAQKKLQEFNARRKLKAAVKAVVASSRLGSASSSHTSIQENHKASSDPPSTQDAKDSTDLLGKKMQEEDQEEDQVEAEASADEMRKLQSEEVEKDAGVKEEETSSMVPQDPEDELETDDPEMKRDSEEKLKSVEEEMDPMTEEEAPDAGLGVPQQDAIQPEY

SEQ ID NO: 16 - DNA, 954 bp, homo sapiens
atgctcaaagtcacggtgccctcctgctccgcctcgtcctgctcttcggtcaccgccagtgcggccccggggaccgcgagcctcgtcccggattactggatcgacggctccaacagggatgcgctgagcgatttcttcgaggtggagtcggagctgggacggggtgctacatccattgtgtacagatgcaaacagaaggggacccagaagccttatgctctcaaagtgttaaagaaaacagtggacaaaaaaatcgtaagaactgagataggagttcttcttcgcctctcacatccaaacattataaaacttaaagagatatttgaaacccctacagaaatcagtctggtcctagaactcgtcacaggaggagaactgtttgataggattgtggaaaagggatattacagtgagcgagatgctgcagatgccgttaaacaaatcctggaggcagttgcttatctacatgaaaatgggattgtccatcgtgatctcaaaccagagaatcttctttatgcaactccagccccagatgcaccactcaaaatcgctgattttggactctctaaaattgtggaacatcaagtgctcatgaagacagtatgtggaaccccagggtactgcgcacctgaaattcttagaggttgtgcctatggacctgaggtggacatgtggtctgtaggaataatcacctacatcttactttgtggatttgaaccattctatgatgaaagaggcgatcagttcatgttcaggagaattctgaattgtgaatattactttatctccccctggtgggatgaagtatctctaaatgccaaggacttggtcagaaaattaattgttttggatccaaagaaacggctgactacatttcaagctctccagcatccgtgggtcacaggtaaagcagccaattttgtacacatggataccgctcaaaagaagctctaa

SEQ ID NO: 17 - protein, 317 aa, homo sapiens
MLKVTVPSCSASSCSSVTASAAPGTASLVPDYWIDGSNRDALSDFFEVESELGRGATSIVYRCKQKGTQKPYALKVLKKTVDKKIVRTEIGVLLRLSHPNIIKLKEIFETPTEISLVLELVTGGELFDRIVEKGYYSERDAADAVKQILEAVAYLHENGIVHRDLKPENLLYATPAPDAPLKIADFGLSKIVEHQVLMKTVCGTPGYCAPEILRGCAYGPEVDMWSVGIITYILLCGFEPFYDERGDQFMFRRILNCEYYFISPWWDEVSLNAKDLVRKLIVLDPKKRLTTFQALQHPWVTGKAANFVHMDTAQKKL

SEQ ID NO: 18 - DNA, 1422 bp, homo sapiens
atgctcaaagtcacggtgccctcctgctccgcctcgtcctgctcttcggtcaccgccagtgcggccccggggaccgcgagcctcgtcccggattactggatcgacggctccaacagggatgcgctgagcgatttcttcgaggtggagtcggagctgggacggggtgctacatccattgtgtacagatgcaaacagaaggggacccagaagccttatgctctcaaagtgttaaagaaaacagtggacaaaaaaatcgtaagaactgagataggagttcttcttcgcctctcacatccaaacattataaaacttaaagagatatttgaaacccctacagaaatcagtctggtcctagaactcgtcacaggaggagaactgtttgataggattgtggaaaagggatattacagtgagcgagatgctgcagatgccgttaaacaaatcctggaggcagttgcttatctacatgaaaatgggattgtccatcgtgatctcaaaccagagaatcttctttatgcaactccagccccagatgcaccactcaaaatcgctgattttggactctctaaaattgtggaacatcaagtgctcatgaagacagtatgtggaaccccagggtactgcgcacctgaaattcttagaggttgtgcctatggacctgaggtggacatgtggtctgtaggaataatcacctacatcttactttgtggatttgaaccattctatgatgaaagaggcgatcagttcatgttcaggagaattctgaattgtgaatattactttatctccccctggtgggatgaagtatctctaaatgccaaggacttggtcagaaaattaattgttttggatccaaagaaacggctgactacatttcaagctctccagcatccgtgggtcacaggtaaagcagccaattttgtacacatggataccgctcaaaagaagctccaagaattcaatgcccggcgtaagcttaaggcagcggtgaaggctgtggtggcctcttcgcgcctgggaagtgccagcagcagccatggcagcatccaggagagccacaaggctagccgagacccttctccaatccaagatggcaacgaggacatgaaagctattccagaaggagagaaaattcaaggcgatggggcccaagccgcagttaagggggcacaggctgagctgatgaaggtgcaagccttagagaaagttaaaggtgcagatataaatgctgaagaggcccccaaaatggtgcccaaggcagtggaggatgggataaaggtggctgacctggaactagaggagggcctagcagaggagaagctgaagactgtggaggaggcagcagctcccagagaagggcaaggaagctctgctgtgggttttgaagttccacagcaagatgtgatcctgccagagtactaa

SEQ ID NO: 19 - protein, 473 aa, homo sapiens
MLKVTVPSCSASSCSSVTASAAPGTASLVPDYWIDGSNRDALSDFFEVESELGRGATSIVYRCKQKGTQKPYALKVLKKTVDKKIVRTEIGVLLRLSHPNIIKLKEIFETPTEISLVLELVTGGELFDRIVEKGYYSERDAADAVKQILEAVAYLHENGIVHRDLKPENLLYATPAPDAPLKIADFGLSKIVEHQVLMKTVCGTPGYCAPEILRGCAYGPEVDMWSVGIITYILLCGFEPFYDERGDQFMFRRILNCEYYFISPWWDEVSLNAKDLVRKLIVLDPKKRLTTFQALQHPWVTGKAANFVHMDTAQKKLQEFNARRKLKAAVKAVVASSRLGSASSSHGSIQESHKASRDPSPIQDGNEDMKAIPEGEKIQGDGAQAAVKGAQAELMKVQALEKVKGADINAEEAPKMVPKAVEDGIKVADLELEEGLAEEKLKTVEEAAAPREGQGSSAVGFEVPQQDVILPEY

SEQ ID NO: 20 - DNA, 876 bp, Rattus norvegicus
atggccaccacggtgacctgcacccgtttcacggacgagtaccagctatacgaggatattggcaagggggctttctctgtggtccgacgctgtgtcaagctctgcaccggccatgagtatgcagctaagatcattaacaccaagaagctgtcagctagagatcaccagaagctggagagggaggctcggatctgccgcctgctgaagcattccaacattgtacgcctccatgacagcatctctgaagagggcttccactacctggtcttcgacctggtcactggtggggagctctttgaagacattgtggcgagagagtactacagtgaggctgacgccagtcactgtatccagcagatcctggaggctgttctccattgtcaccaaatgggggtcgtccacagagacctcaagcctgaaaacctgctcctggccagcaaatgcaaaggggccgcagtgaaactggcagacttcggcctggccatcgaggttcagggagaccagcaggcatggtttggatttgcgggaacaccaggctacctgtctcccgaagttcttcggaaggaggcctatggcaaaccagtggatatctgggcatgtggggtgatcctgtatatcctgctggtgggatacccacctttctgggatgaggaccagcacaagctgtaccagcagatcaaggctggggcctatgacttcccatcccccgagtgggacaccgttacccctgaagccaaaaacctcatcaaccagatgttgaccatcaaccccgccaagcgcatcacggcccacgaggccctgaagcacccatgggtctgccaacgatccacggtggcctccatgatgcacagacaggagactgtggaatgtctgtga

SEQ ID NO: 21 - protein, 291 aa, Rattus norvegicus
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECL

SEQ ID NO: 22 - DNA, 1629 bp, Rattus norvegicus
atggccaccacggtgacctgcacccgtttcacggacgagtaccagctatacgaggatattggcaagggggctttctctgtggtccgacgctgtgtcaagctctgcaccggccatgagtatgcagctaagatcattaacaccaagaagctgtcagctagagatcaccagaagctggagagggaggctcggatctgccgcctgctgaagcattccaacattgtacgcctccatgacagcatctctgaagagggcttccactacctggtcttcgacctggtcactggtggggagctctttgaagacattgtggcgagagagtactacagtgaggctgacgccagtcactgtatccagcagatcctggaggctgttctccattgtcaccaaatgggggtcgtccacagagacctcaagcctgaaaacctgctcctggccagcaaatgcaaaggggccgcagtgaaactggcagacttcggcctggccatcgaggttcagggagaccagcaggcatggtttggatttgcgggaacaccaggctacctgtctcccgaagttcttcggaaggaggcctatggcaaaccagtggatatctgggcatgtggggtgatcctgtatatcctgctggtgggatacccacctttctgggatgaggaccagcacaagctgtaccagcagatcaaggctggggcctatgacttcccatcccccgagtgggacaccgttacccctgaagccaaaaacctcatcaaccagatgttgaccatcaaccccgccaagcgcatcacggcccacgaggccctgaagcacccatgggtctgccaacgatccacggtggcctccatgatgcacagacaggaggatgtggaatgtctgaagaagttcaatgcaaggaggaagctcaagggagccatcctcaccactatgctggccacacggaatttctcagtgggcagacagaccaccgctccggccacaatgtccaccgcggcctccggcaccaccatggggctggtggaacaagccaagagtttactcaacaagaaagcagacggagtcaagccccagacaaacagcaccaaaaacagctcggccatcaccagccccaaaggatccctccctcctgccgccctggagcctcaaaccaccgttatccataacccagtggacggcattaaggaatcttccgacagcaccaacacaaccatagaggacgaagatgccaaagcccggaagcaggaaatcatcaagaccacagagcagctcatcgaggccgtcaacaacggcgactttgaggcctatgcgaaaatctgtgacccaggcctgacctcatttgagcctgaagctctgggcaacctggtcgaagggatggatttccacagattctactttgagaacctgctggccaagaacagcaagccgatccacaccactatcctgaacccgcacgtgcacgtcatcggcgaggatgcagcctgcatcgcttacatccgcctcacacagtacatcgacggccagggcagaccccgcaccagccagtccgaagagacccgtgtgtggcaccgccgcgacggcaagtggcagaatgtccatttccactgctcgggcgctccagtggccccactgcagtga

SEQ ID NO: 23 - protein, 542 aa, Rattus norvegicus
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQEDVECLKKFNARRKLKGAILTTMLATRNFSVGRQTTAPATMSTAASGTTMGLVEQAKSLLNKKADGVKPQTNSTKNSSAITSPKGSLPPAALEPQTTVIHNPVDGIKESSDSTNTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 24 - DNA, 1629 bp, Rattus norvegicus
atggccaccacggtgacctgcacccgtttcacggacgagtaccagctatacgaggatattggcaagggggctttctctgtggtccgacgctgtgtcaagctctgcaccggccatgagtatgcagctaagatcattaacaccaagaagctgtcagctagagatcaccagaagctggagagggaggctcggatctgccgcctgctgaagcattccaacattgtacgcctccatgacagcatctctgaagagggcttccactacctggtcttcgacctggtcactggtggggagctctttgaagacattgtggcgagagagtactacagtgaggctgacgccagtcactgtatccagcagatcctggaggctgttctccattgtcaccaaatgggggtcgtccacagagacctcaagcctgaaaacctgctcctggccagcaaatgcaaaggggccgcagtgaaactggcagacttcggcctggccatcgaggttcagggagaccagcaggcatggtttggatttgcgggaacaccaggctacctgtctcccgaagttcttcggaaggaggcctatggcaaaccagtggatatctgggcatgtggggtgatcctgtatatcctgctggtgggatacccacctttctgggatgaggaccagcacaagctgtaccagcagatcaaggctggggcctatgacttcccatcccccgagtgggacaccgttacccctgaagccaaaaacctcatcaaccagatgttgaccatcaaccccgccaagcgcatcacggcccacgaggccctgaagcacccatgggtctgccaacgatccacggtggcctccatgatgcacagacaggagactgtggaatgtctgaagaagttcaatgcaaggaggaagctcaagggagccatcctcaccactatgctggccacacggaatttctcagtgggcagacagaccaccgctccggccacaatgtccaccgcggcctccggcaccaccatggggctggtggaacaagccaagagtttactcaacaagaaagcagacggagtcaagccccagacaaacagcaccaaaaacagctcggccatcaccagccccaaaggatccctccctcctgccgccctggagcctcaaaccaccgttatccataacccagtggacggcattaaggaatcttccgacagcaccaacacaaccatagaggacgaagatgccaaagcccggaagcaggaaatcatcaagaccacagagcagctcatcgaggccgtcaacaacggcgactttgaggcctatgcgaaaatctgtgacccaggcctgacctcatttgagcctgaagctctgggcaacctggtcgaagggatggatttccacagattctactttgagaacctgctggccaagaacagcaagccgatccacaccactatcctgaacccgcacgtgcacgtcatcggcgaggatgcagcctgcatcgcttacatccgcctcacacagtacatcgacggccagggcagaccccgcaccagccagtccgaagagacccgtgtgtggcaccgccgcgacggcaagtggcagaatgtccatttccactgctcgggcgctccagtggccccactgcagtga

SEQ ID NO: 25 - protein, 542 aa, Rattus norvegicus
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSVGRQTTAPATMSTAASGTTMGLVEQAKSLLNKKADGVKPQTNSTKNSSAITSPKGSLPPAALEPQTTVIHNPVDGIKESSDSTNTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 26 - DNA, 1437 bp, Rattus norvegicus
atggctaccatcacctgcacccgattcacggaagagtaccagctcttcgaggaactgggaaagggagccttctccgtggtgcgcaggtgtgtgaaggtgctggctggccaggagtatgctgccaagattatcaacaccaagaagctctcagccagagatcaccagaagttggaacgcgaggcccgcatctgccgcttgttgaagcaccccaatatcgtccgactccatgacagcatctccgaggaggggcaccactaccttatcttcgatctggtcactggtggggagctgttcgaagacattgtggcccgggagtattacagtgaggctgatgccagccactgtatccagcagatcctggaggctgtgctacactgtcaccagatgggggtggtgcatcgcgacctgaagcctgagaatctgttgctggcttcgaagctcaagggtgctgcggtgaagctggcagactttggcctggccatagaggttgagggagagcagcaggcatggtttgggttcgcagggacacctggatacctctccccagaagtgctgcggaaggacccatacgggaagcctgtggacctgtgggcctgtggcgtcatcctgtatatcttgctggttgggtatcccccattctgggatgaggaccagcaccgcctgtaccagcagatcaaagctggtgcctacgatttcccatcaccagaatgggacaccgtcaccccggaagccaaggatctgatcaataagatgctgaccatcaacccgtccaaacgcatcacggccgctgaggctctcaagcacccctggatctcgcaccgctccactgtggcctcctgcatgcacagacaggaggacgtggactgcctgaagaagttcaatgccaggaggaaactgaagggagccatcctcaccactatgctggccaccaggaacttctccggagggaagagtggaggaaacaagaagaatgatggcgtgaaggaatcctctgagagcaccaacaccaccatcgaggatgaagacaccaaagtgcgcaaacaggaaattatcaaagtgacagagcagctgatcgaagccataagcaatggagactttgaatcctacacgaagatgtgcgaccctggaatgacagcctttgaaccggaggccctggggaacctggtcgagggcctggactttcatcgattctattttgaaaacctgtggtcccggaacagcaagcccgtgcacaccaccatcctgaaccctcacatccacctgatgggtgacgagtcagcctgcatcgcctacatccgcatcactcagtacctggatgcgggtggcatcccccgcacggcccagtcagaggagacccgtgtctggcaccgcagggatggaaaatggcagatcgtccacttccacagatctggggcgccctccgtcctgccccattga

SEQ ID NO: 27 - protein, 478 aa, Rattus norvegicus
MATITCTRFTEEYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQEDVDCLKKFNARRKLKGAILTTMLATRNFSGGKSGGNKKNDGVKESSESTNTTIEDEDTKVRKQEIIKVTEQLIEAISNGDFESYTKMCDPGMTAFEPEALGNLVEGLDFHRFYFENLWSRNSKPVHTTILNPHIHLMGDESACIAYIRITQYLDAGGIPRTAQSEETRVWHRRDGKWQIVHFHRSGAPSVLPH

SEQ ID NO: 28 - DNA, 1437 bp, Rattus norvegicus
atggctaccatcacctgcacccgattcacggaagagtaccagctcttcgaggaactgggaaagggagccttctccgtggtgcgcaggtgtgtgaaggtgctggctggccaggagtatgctgccaagattatcaacaccaagaagctctcagccagagatcaccagaagttggaacgcgaggcccgcatctgccgcttgttgaagcaccccaatatcgtccgactccatgacagcatctccgaggaggggcaccactaccttatcttcgatctggtcactggtggggagctgttcgaagacattgtggcccgggagtattacagtgaggctgatgccagccactgtatccagcagatcctggaggctgtgctacactgtcaccagatgggggtggtgcatcgcgacctgaagcctgagaatctgttgctggcttcgaagctcaagggtgctgcggtgaagctggcagactttggcctggccatagaggttgagggagagcagcaggcatggtttgggttcgcagggacacctggatacctctccccagaagtgctgcggaaggacccatacgggaagcctgtggacctgtgggcctgtggcgtcatcctgtatatcttgctggttgggtatcccccattctgggatgaggaccagcaccgcctgtaccagcagatcaaagctggtgcctacgatttcccatcaccagaatgggacaccgtcaccccggaagccaaggatctgatcaataagatgctgaccatcaacccgtccaaacgcatcacggccgctgaggctctcaagcacccctggatctcgcaccgctccactgtggcctcctgcatgcacagacaggagaccgtggactgcctgaagaagttcaatgccaggaggaaactgaagggagccatcctcaccactatgctggccaccaggaacttctccggagggaagagtggaggaaacaagaagaatgatggcgtgaaggaatcctctgagagcaccaacaccaccatcgaggatgaagacaccaaagtgcgcaaacaggaaattatcaaagtgacagagcagctgatcgaagccataagcaatggagactttgaatcctacacgaagatgtgcgaccctggaatgacagcctttgaaccggaggccctggggaacctggtcgagggcctggactttcatcgattctattttgaaaacctgtggtcccggaacagcaagcccgtgcacaccaccatcctgaaccctcacatccacctgatgggtgacgagtcagcctgcatcgcctacatccgcatcactcagtacctggatgcgggtggcatcccccgcacggcccagtcagaggagacccgtgtctggcaccgcagggatggaaaatggcagatcgtccacttccacagatctggggcgccctccgtcctgccccattga

SEQ ID NO: 29 - protein, 478 aa, Rattus norvegicus
MATITCTRFTEEYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQETVDCLKKFNARRKLKGAILTTMLATRNFSGGKSGGNKKNDGVKESSESTNTTIEDEDTKVRKQEIIKVTEQLIEAISNGDFESYTKMCDPGMTAFEPEALGNLVEGLDFHRFYFENLWSRNSKPVHTTILNPHIHLMGDESACIAYIRITQYLDAGGIPRTAQSEETRVWHRRDGKWQIVHFHRSGAPSVLPH

SEQ ID NO: 30 - DNA, 873 bp, Rattus norvegicus
atggctaccatcacctgcacccgattcacggaagagtaccagctcttcgaggaactgggaaagggagccttctccgtggtgcgcaggtgtgtgaaggtgctggctggccaggagtatgctgccaagattatcaacaccaagaagctctcagccagagatcaccagaagttggaacgcgaggcccgcatctgccgcttgttgaagcaccccaatatcgtccgactccatgacagcatctccgaggaggggcaccactaccttatcttcgatctggtcactggtggggagctgttcgaagacattgtggcccgggagtattacagtgaggctgatgccagccactgtatccagcagatcctggaggctgtgctacactgtcaccagatgggggtggtgcatcgcgacctgaagcctgagaatctgttgctggcttcgaagctcaagggtgctgcggtgaagctggcagactttggcctggccatagaggttgagggagagcagcaggcatggtttgggttcgcagggacacctggatacctctccccagaagtgctgcggaaggacccatacgggaagcctgtggacctgtgggcctgtggcgtcatcctgtatatcttgctggttgggtatcccccattctgggatgaggaccagcaccgcctgtaccagcagatcaaagctggtgcctacgatttcccatcaccagaatgggacaccgtcaccccggaagccaaggatctgatcaataagatgctgaccatcaacccgtccaaacgcatcacggccgctgaggctctcaagcacccctggatctcgcaccgctccactgtggcctcctgcatgcacagacaggagaccgtggactgcctgtga

SEQ ID NO: 31 - protein, 290 aa, Rattus norvegicus
MATITCTRFTEEYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQETVDCL

SEQ ID NO: 32 - DNA, 1629 bp, homo sapiens
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggaggatgtggagtgtctgaaaaagttcaatgccaggagaaagctcaagggagccatcctcaccaccatgctggccacacggaatttctcagtgggcagacagaccaccgctccggccacaatgtccaccgcggcctccggcaccaccatggggctggtggaacaagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtga

SEQ ID NO: 33 - protein, 542 aa, homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQEDVECLKKFNARRKLKGAILTTMLATRNFSVGRQTTAPATMSTAASGTTMGLVEQAKSLLNKKADGVKPQTNSTKNSAAATSPKGTLPPAALEPQTTVIHNPVDGIKESSDSANTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 34 - DNA, 1629 bp, homo sapiens
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagtgggcagacagaccaccgctccggccacaatgtccaccgcggcctccggcaccaccatggggctggtggaacaagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtga

SEQ ID NO: 35 - protein, 542 aa, homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSVGRQTTAPATMSTAASGTTMGLVEQAKSLLNKKADGVKPQTNSTKNSAAATSPKGTLPPAALEPQTTVIHNPVDGIKESSDSANTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 36 - DNA, 1437 bp, homo sapiens
atggccaccatcacctgcacccgcttcacggaagagtaccagctcttcgaggaattgggcaagggagccttctcggtggtgcgaaggtgtgtgaaggtgctggctggccaggagtatgctgccaagatcatcaacacaaagaagctgtcagccagagaccatcagaagctggagcgtgaagcccgcatctgccgcctgctgaagcaccccaacatcgtccgactacatgacagcatctcagaggagggacaccactacctgatcttcgacctggtcactggtggggaactgtttgaagatatcgtggcccgggagtattacagtgaggcggatgccagtcactgtatccagcagatcctggaggctgtgctgcactgccaccagatgggggtggtgcaccgggacctgaagcctgagaatctgttgctggcctccaagctcaagggtgccgcagtgaagctggcagactttggcctggccatagaggtggagggggagcagcaggcatggtttgggtttgcagggactcctggatatctctccccagaagtgctgcggaaggacccgtacgggaagcctgtggacctgtgggcttgtggggtcatcctgtacatcctgctggttgggtaccccccgttctgggatgaggaccagcaccgcctgtaccagcagatcaaagccggcgcctatgatttcccatcgccggaatgggacactgtcaccccggaagccaaggatctgatcaataagatgctgaccattaacccatccaaacgcatcacagctgccgaagcccttaagcacccctggatctcgcaccgctccaccgtggcatcctgcatgcacagacaggaggacgtggactgcctgaagaagttcaatgccaggaggaaactgaagggagccattctcaccacgatgctggccaccaggaacttctccggagggaagagtgggggaaacaagaagagcgatggtgtgaaggaatcctcagagagcaccaacaccaccatcgaggatgaagacaccaaagtgcggaaacaggaaattataaaagtgacagagcagctgattgaagccataagcaatggagattttgagtcctacacgaagatgtgcgaccctggcatgacagccttcgaacctgaggccctggggaacctggttgagggcctggacttccatcgattctattttgaaaacctgtggtcccggaacagcaagcccgtgcacaccaccatcctgaatccccacatccacctgatgggcgacgagtcagcctgcatcgcctacatccgcatcacgcagtacctggacgctggcggcatcccacgcaccgcccagtcggaggagacccgtgtctggcaccgccgggatggcaaatggcagatcgtccacttccacagatctggggcgccctccgtcctgccccactga

SEQ ID NO: 37 - protein, 478 aa, homo sapiens
MATITCTRFTEEYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQEDVDCLKKFNARRKLKGAILTTMLATRNFSGGKSGGNKKSDGVKESSESTNTTIEDEDTKVRKQEIIKVTEQLIEAISNGDFESYTKMCDPGMTAFEPEALGNLVEGLDFHRFYFENLWSRNSKPVHTTILNPHIHLMGDESACIAYIRITQYLDAGGIPRTAQSEETRVWHRRDGKWQIVHFHRSGAPSVLPH

SEQ ID NO: 38 - DNA, 1437 bp, homo sapiens
atggccaccatcacctgcacccgcttcacggaagagtaccagctcttcgaggaattgggcaagggagccttctcggtggtgcgaaggtgtgtgaaggtgctggctggccaggagtatgctgccaagatcatcaacacaaagaagctgtcagccagagaccatcagaagctggagcgtgaagcccgcatctgccgcctgctgaagcaccccaacatcgtccgactacatgacagcatctcagaggagggacaccactacctgatcttcgacctggtcactggtggggaactgtttgaagatatcgtggcccgggagtattacagtgaggcggatgccagtcactgtatccagcagatcctggaggctgtgctgcactgccaccagatgggggtggtgcaccgggacctgaagcctgagaatctgttgctggcctccaagctcaagggtgccgcagtgaagctggcagactttggcctggccatagaggtggagggggagcagcaggcatggtttgggtttgcagggactcctggatatctctccccagaagtgctgcggaaggacccgtacgggaagcctgtggacctgtgggcttgtggggtcatcctgtacatcctgctggttgggtaccccccgttctgggatgaggaccagcaccgcctgtaccagcagatcaaagccggcgcctatgatttcccatcgccggaatgggacactgtcaccccggaagccaaggatctgatcaataagatgctgaccattaacccatccaaacgcatcacagctgccgaagcccttaagcacccctggatctcgcaccgctccaccgtggcatcctgcatgcacagacaggagaccgtggactgcctgaagaagttcaatgccaggaggaaactgaagggagccattctcaccacgatgctggccaccaggaacttctccggagggaagagtgggggaaacaagaagagcgatggtgtgaaggaatcctcagagagcaccaacaccaccatcgaggatgaagacaccaaagtgcggaaacaggaaattataaaagtgacagagcagctgattgaagccataagcaatggagattttgagtcctacacgaagatgtgcgaccctggcatgacagccttcgaacctgaggccctggggaacctggttgagggcctggacttccatcgattctattttgaaaacctgtggtcccggaacagcaagcccgtgcacaccaccatcctgaatccccacatccacctgatgggcgacgagtcagcctgcatcgcctacatccgcatcacgcagtacctggacgctggcggcatcccacgcaccgcccagtcggaggagacccgtgtctggcaccgccgggatggcaaatggcagatcgtccacttccacagatctggggcgccctccgtcctgccccactga

SEQ ID NO: 39 - protein, 478 aa, homo sapiens
MATITCTRFTEEYQLFEELGKGAFSVVRRCVKVLAGQEYAAKIINTKKLSARDHQKLEREARICRLLKHPNIVRLHDSISEEGHHYLIFDLVTGGELFEDIVAREYYSEADASHCIQQILEAVLHCHQMGVVHRDLKPENLLLASKLKGAAVKLADFGLAIEVEGEQQAWFGFAGTPGYLSPEVLRKDPYGKPVDLWACGVILYILLVGYPPFWDEDQHRLYQQIKAGAYDFPSPEWDTVTPEAKDLINKMLTINPSKRITAAEALKHPWISHRSTVASCMHRQETVDCLKKFNARRKLKGAILTTMLATRNFSGGKSGGNKKSDGVKESSESTNTTIEDEDTKVRKQEIIKVTEQLIEAISNGDFESYTKMCDPGMTAFEPEALGNLVEGLDFHRFYFENLWSRNSKPVHTTILNPHIHLMGDESACIAYIRITQYLDAGGIPRTAQSEETRVWHRRDGKWQIVHFHRSGAPSVLPH

SEQ ID NO: 40 - DNA, 876 bp, homo sapiens
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgtga

SEQ ID NO: 41 - protein, 291 aa, homo sapiens; identical to SEQ ID NO: 21

SEQ ID NO: 42 - DNA, 873 bp, homo sapiens
atggccaccatcacctgcacccgcttcacggaagagtaccagctcttcgaggaattgggcaagggagccttctcggtggtgcgaaggtgtgtgaaggtgctggctggccaggagtatgctgccaagatcatcaacacaaagaagctgtcagccagagaccatcagaagctggagcgtgaagcccgcatctgccgcctgctgaagcaccccaacatcgtccgactacatgacagcatctcagaggagggacaccactacctgatcttcgacctggtcactggtggggaactgtttgaagatatcgtggcccgggagtattacagtgaggcggatgccagtcactgtatccagcagatcctggaggctgtgctgcactgccaccagatgggggtggtgcaccgggacctgaagcctgagaatctgttgctggcctccaagctcaagggtgccgcagtgaagctggcagactttggcctggccatagaggtggagggggagcagcaggcatggtttgggtttgcagggactcctggatatctctccccagaagtgctgcggaaggacccgtacgggaagcctgtggacctgtgggcttgtggggtcatcctgtacatcctgctggttgggtaccccccgttctgggatgaggaccagcaccgcctgtaccagcagatcaaagccggcgcctatgatttcccatcgccggaatgggacactgtcaccccggaagccaaggatctgatcaataagatgctgaccattaacccatccaaacgcatcacagctgccgaagcccttaagcacccctggatctcgcaccgctccaccgtggcatcctgcatgcacagacaggagaccgtggactgcctgtga

SEQ ID NO: 43 - protein, 290 aa, homo sapiens; identical to SEQ ID NO: 31

SEQ ID NO: 44 - DNA, 882 bp, homo sapiens
atgctgggggcagtggaaggccccaggtggaagcaggcggaggacattagagacatctacgacttccgagatgttctgggcacgggggccttctcggaggtgatcctggcagaagataagaggacgcagaagctggtggccatcaaatgcattgccaaggaggccctggagggcaaggaaggcagcatggagaatgagattgctgtcctgcacaagatcaagcaccccaacattgtagccctggatgacatctatgagagtgggggccacctctacctcatcatgcagctggtgtcgggtggggagctctttgaccgtattgtggaaaaaggcttctacacggagcgggacgccagccgcctcatcttccaggtgctggatgctgtgaaatacctgcatgacctgggcattgtacaccgggatctcaagccagagaatctgctgtactacagcctggatgaagactccaaaatcatgatctccgactttggcctctccaagatggaggacccgggcagtgtgctctccaccgcctgtggaactccgggatacgtggcccctgaagtcctggcccagaagccctacagcaaggctgtggattgctggtccataggtgtcatcgcctacatcttgctctgcggttaccctcccttctatgacgagaatgatgccaaactctttgaacagattttgaaggccgagtacgagtttgactctccttactgggacgacatctctgactctgccaaagatttcatccggcacttgatggagaaggacccagagaaaagattcacctgtgagcaggccttgcagcacccatggattgcaggagatacagctctagataagaatatccaccagtcggtgagtgagcagtag

SEQ ID NO: 45 - protein, 293 aa, homo sapiens
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQ

SEQ ID NO: 46 - DNA, 1113 bp, homo sapiens
atgctgggggcagtggaaggccccaggtggaagcaggcggaggacattagagacatctacgacttccgagatgttctgggcacgggggccttctcggaggtgatcctggcagaagataagaggacgcagaagctggtggccatcaaatgcattgccaaggaggccctggagggcaaggaaggcagcatggagaatgagattgctgtcctgcacaagatcaagcaccccaacattgtagccctggatgacatctatgagagtgggggccacctctacctcatcatgcagctggtgtcgggtggggagctctttgaccgtattgtggaaaaaggcttctacacggagcgggacgccagccgcctcatcttccaggtgctggatgctgtgaaatacctgcatgacctgggcattgtacaccgggatctcaagccagagaatctgctgtactacagcctggatgaagactccaaaatcatgatctccgactttggcctctccaagatggaggacccgggcagtgtgctctccaccgcctgtggaactccgggatacgtggcccctgaagtcctggcccagaagccctacagcaaggctgtggattgctggtccataggtgtcatcgcctacatcttgctctgcggttaccctcccttctatgacgagaatgatgccaaactctttgaacagattttgaaggccgagtacgagtttgactctccttactgggacgacatctctgactctgccaaagatttcatccggcacttgatggagaaggacccagagaaaagattcacctgtgagcaggccttgcagcacccatggattgcaggagatacagctctagataagaatatccaccagtcggtgagtgagcagatcaagaagaactttgccaagagcaagtggaagcaagccttcaatgccacggctgtggtgcggcacatgaggaaactgcagctgggcaccagccaggaggggcaggggcagacggcgagccatggggagctgctgacaccagtggctggggggccggcagctggctgttgctgtcgagactgctgcgtggagccgggcacagaactgtcccccacactgccccaccagctctag

SEQ ID NO: 47 - protein, 370 aa, homo sapiens
MLGAVEGPRWKQAEDIRDIYDFRDVLGTGAFSEVILAEDKRTQKLVAIKCIAKEALEGKEGSMENEIAVLHKIKHPNIVALDDIYESGGHLYLIMQLVSGGELFDRIVEKGFYTERDASRLIFQVLDAVKYLHDLGIVHRDLKPENLLYYSLDEDSKIMISDFGLSKMEDPGSVLSTACGTPGYVAPEVLAQKPYSKAVDCWSIGVIAYILLCGYPPFYDENDAKLFEQILKAEYEFDSPYWDDISDSAKDFIRHLMEKDPEKRFTCEQALQHPWIAGDTALDKNIHQSVSEQIKKNFAKSKWKQAFNATAVVRHMRKLQLGTSQEGQGQTASHGELLTPVAGGPAAGCCCRDCCVEPGTELSPTLPHQL

(19) United States
(12) Patent Application Publication
(10) Pub. No.: US 2025/0018058 A1
(43) Pub. Date: Jan. 16, 2025

(54) METHOD FOR DECREASING DEGENERATION OF RETINAL GANGLION CELLS

(71) Applicant: ICAHN SCHOOL OF MEDICINE AT MOUNT SINAI, New York, NY (US)

(72) Inventors: Bo CHEN, New York, NY (US); Xinzheng GUO, New York (NY)

(73) Assignee: ICAHN SCHOOL OF MEDICINE AT MOUNT SINAI, New York, NY (US)

(21) Appl. No.: 18/547,930

(22) PCT Filed: Feb. 25, 2022
(86) PCT No.: PCT/US2022/017894

§ 371 Date: Aug. 25, 2023

Related U.S. Application Data
(60) Provisional application No. 63/154,432, filed on Feb. 26, 2021.
(60) Provisional application No. 63/177,230, filed on Apr. 20, 2021.

(51) Int. Cl. A61K 48/00; A61K 38/45; A61P 27/06

(52) U.S. Cl. CPC A61K 48/0058; A61K 38/45; A61K 48/0075; A61P 27/06

(57) ABSTRACT

A method of decreasing degeneration of retinal ganglion cells in a subject is provided, including administering to the subject a composition to increase activity of a calcium-calmodulin dependent kinase (CaMK) or cyclic-AMP response element-binding protein (CREB), wherein the composition comprises the CaMK or CREB or a polynucleotide encoding the CaMK or the CREB. Also provided is a method for treating vision loss in a subject is provided, including administering to the subject a composition to increase activity of a CaMK or CREB, wherein the composition comprises the CaMK or CREB or a polynucleotide encoding the CaMK or the CREB. Also provided is a pharmaceutical composition, including a polynucleotide and a vector, wherein the polynucleotide includes a retinal ganglion cell promoter and encodes a CaMK or CREB.

### CROSS-REFERENCE TO RELATED APPLICATIONS

This application is an International Application filed pursuant to the Patent Cooperation treaty, and claims benefit of priority from U.S. Provisional Patent Application No. 63/154,432, filed Feb. 26, 2021, and U.S. Provisional Patent Application No. 63/177,230, filed Apr. 20, 2021, the entire contents of which are incorporated herein by reference.

### GOVERNMENT RIGHTS STATEMENT

This disclosure was made with Government support under grant number EY028921 awarded by the National Institutes of Health. The Government has certain rights in the disclosure.

### SEQUENCE LISTING

The instant application contains a Sequence Listing, created on Feb. 3, 2022; the file, in ASCII format, is designated H2257236.txt and is 119.4 KB in size. The file is hereby incorporated by reference in its entirety into the instant application. The sequence listing submitted herewith is identical to the sequence listing forming part of the international application.

### BACKGROUND

Visual information is transmitted from the eye to higher processing centers in the brain via the optic nerve, a bundle of axons emerging from the retina's output neurons: the retinal ganglion cells (RGCs). The loss of RGCs is a leading cause of visual impairment and blindness in a variety of pathological states. Some conditions injure the RGC soma, including excitotoxicity and retinal ischemia, whereas others injure the RGC axon, including optic nerve transection, compression, papilledema and glaucoma. Indeed, glaucoma is the leading cause of irreversible visual impairment worldwide.

A barrier to restoring vision following RGC injury is inducing axons to regenerate. There is unmet clinical needs to develop effective neuroprotective approaches to preserve RGCs and their function. Early neuroprotective treatment is required to prevent acute and massive RGC loss for high-risk individuals of retinal ischemia and excitotoxicity. RGC neuroprotective intervention is also required for a significant proportion of glaucoma patients who still progress to blindness despite treatment to reduce intraocular pressure. For patients with traumatic optic nerve injury, promoting RGC survival is may aid efforts to regenerate retina-brain connections.

The present disclosure is directed to overcoming these and other deficiencies in the art.

### SUMMARY

It is therefore desirable to identify a therapeutic approach for vision impairment involving RGC degeneration or loss, such as identifying a target that is effective in protecting RGC somas and axons from diverse insults in a wide spectrum of pathological. The present disclosure includes identification of pharmaceutical compositions that increase activity of Ca2+/Calmodulin dependent protein kinase and use thereof in treating RGC degeneration or loss and in treating vision impairment, surprising in view of existing literature demonstrating that CaMK activity promotes excitotoxic cell death, including in RGCs. The present disclosure also includes identification of pharmaceutical compositions that increase activity of cyclic-AMP response element-binding protein (CREB) as use in treating RGC degeneration or loss and in treating vision impairment

Herein, in an aspect, provided is a method of decreasing degeneration of retinal ganglion cells in a subject, including administering to the subject a composition to increase activity of a CaMK, wherein the composition includes the CaMK or a polynucleotide encoding the CaMK. In an example, the composition further includes a vector, for example a viral vector. The viral vector may include an adeno-associated viral vector (AAV).

In another example, the CaMK is selected from one or more of CaMKI, CaMKII, and CaMKIV. In a further example, the CaMK is selected from one or more of CaMKIIα, CaMKIIβ, CaMKIIγ, and CaMKIIδ. In still another example, the CaMK is constitutively active. In still a further example, the CaMKII is selected from one or both of a CaMKIIα comprising a T286D substitution and a CaMKIIβ comprising a T287D substitution.

In another example, the composition includes a polynucleotide encoding the CaMK. In a further example, the polynucleotide further includes a retinal ganglion cell promoter. In yet another example, the retinal ganglion cell promoter includes a gamma-Synuclein promoter, or a Synapsin 1 promoter, or a Neurofilament Heavy promoter, or a Thy-1 cell surface antigen promoter. In still another example, the retinal ganglion cell promoter includes a gamma-Synuclein promoter. In yet another example, the composition includes the CaMK.

In another example, the administering is selected from intraocular administration and systemic administration. In yet another example, the subject has or is at risk for having one or more of glaucoma, diabetic retinopathy, retinal ischemia, and optic nerve injury. In still another example, preventing degeneration includes preventing reduction of one or both of retinal ganglion cell somata and retinal ganglion call axons.

In another aspect, provided is a method of treating vision loss in a subject, including administering to the subject a composition to increase activity of a CaMK, wherein the composition includes the CaMK or a polynucleotide encoding the CaMK. In an example, the composition further includes a vector, for example a viral vector. The viral vector may include an AAV.

In another example, the CaMK is selected from one or more of CaMKI, CaMKII, and CaMKIV. In a further example, the CaMK is selected from one or more of CaMKIIα, CaMKIIβ, CaMKIIγ, and CaMKIIδ. In still another example, the CaMK is constitutively active. In still a further example, the CaMKII is selected from one or both of a CaMKIIα comprising a T286D substitution and a CaMKIIβ comprising a T287D substitution.

In another example, the composition includes a polynucleotide encoding the CaMK. In a further example, the polynucleotide further includes a retinal ganglion cell promoter. In yet another example, the retinal ganglion cell promoter includes a gamma-Synuclein promoter, or a Synapsin 1 promoter, or a Neurofilament Heavy promoter, or a Thy-1 cell surface antigen promoter. In still another example, the retinal ganglion cell promoter includes a gamma-Synuclein promoter. In yet another example, the composition includes the CaMK.

In another example, the administering is selected from intraocular administration and systemic administration. In yet another example, the subject has or is at risk for having one or more of glaucoma, diabetic retinopathy, retinal ischemia, and optic nerve injury. In still another example, preventing degeneration includes preventing reduction of one or both of retinal ganglion cell somata and retinal ganglion call axons. In still another example, treating includes preventing vision loss.

In yet another aspect, provided is a pharmaceutical composition, including a polynucleotide and a vector, wherein the polynucleotide includes a retinal ganglion cell promoter and encodes a CaMK. In yet another example, the retinal ganglion cell promoter includes a gamma-Synuclein promoter, or a Synapsin 1 promoter, or a Neurofilament Heavy promoter, or a Thy-1 cell surface antigen promoter. In an example, the retinal ganglion cell promoter includes a gamma-Synuclein promoter. In another example, the vector includes a viral vector. In yet another example, the vector includes an adeno-associated vector. In a further example, the CaMK is selected from one or more of CaMKI, CaMKII, and CaMKIV. In yet a further example, the CaMK is selected from one or more of CaMKIIα, CaMKIIβ, CaMKIIγ, and CaMKIIδ.

In another example, the CaMK is constitutively active. In still another example, the CaMKII is selected from one or both of a CaMKIIα comprising a T286D substitution and a CaMKIIβ comprising a T287D substitution.

In still another aspect, provided is a method of decreasing degeneration of retinal ganglion cells in a subject, including administering to the subject a composition to increase activity of a CREB, wherein the composition includes the CREB or a polynucleotide encoding the CREB. In an example, the composition further includes a vector, for example a viral vector. The viral vector may include an adeno-associated viral vector (AAV).

In another example, the CREB is constitutively active. In still another example, the CREB includes VP-16 CREB. In yet another example, the composition includes a polynucleotide encoding the CREB. In a further example, the polynucleotide further includes a retinal ganglion cell promoter. In yet another example, the retinal ganglion cell promoter includes a gamma-Synuclein promoter, or a Synapsin 1 promoter, or a Neurofilament Heavy promoter, or a Thy-1 cell surface antigen promoter. In still another example, the retinal ganglion cell promoter includes a gamma-Synuclein promoter. In yet another example, the composition includes the CREB.

In another example, the administering is selected from intraocular administration and systemic administration. In yet another example, the subject has or is at risk for having one or more of glaucoma, diabetic retinopathy, retinal ischemia, and optic nerve injury. In still another example, preventing degeneration includes preventing reduction of one or both of retinal ganglion cell somata and retinal ganglion call axons.

In another aspect, provided is a method of treating vision loss in a subject, including administering to the subject a composition to increase activity of a CREB, wherein the composition includes the CREB or a polynucleotide encoding the CREB. In an example, the composition further includes a vector, for example a viral vector. The viral vector may include an AAV.

In another example, the CREB is constitutively active. In still another example, the CREB includes VP-16 CREB. In yet another example, the composition includes a polynucleotide encoding the CREB. In a further example, the polynucleotide further includes a retinal ganglion cell promoter. In yet another example, the retinal ganglion cell promoter includes a gamma-Synuclein promoter, or a Synapsin 1 promoter, or a Neurofilament Heavy promoter, or a Thy-1 cell surface antigen promoter. In still another example, the retinal ganglion cell promoter includes a gamma-Synuclein promoter. In yet another example, the composition includes the CREB.

In another example, the composition includes a polynucleotide encoding the CREB. In a further example, the polynucleotide further includes a retinal ganglion cell promoter. In yet another example, the retinal ganglion cell promoter includes a gamma-Synuclein promoter, or a Synapsin 1 promoter, or a Neurofilament Heavy promoter, or a Thy-1 cell surface antigen promoter. In still another example, the retinal ganglion cell promoter includes a gamma-Synuclein promoter. In yet another example, the composition includes the CREB.

In another example, the administering is selected from intraocular administration and systemic administration. In yet another example, the subject has or is at risk for having one or more of glaucoma, diabetic retinopathy, retinal ischemia, and optic nerve injury. In still another example, preventing degeneration includes preventing reduction of one or both of retinal ganglion cell somata and retinal ganglion call axons. In still another example, treating includes preventing vision loss.

In yet another aspect, provided is a pharmaceutical composition, including a polynucleotide and a vector, wherein the polynucleotide includes a retinal ganglion cell promoter and encodes a CREB. In yet another example, the retinal ganglion cell promoter includes a gamma-Synuclein promoter, or a Synapsin 1 promoter, or a Neurofilament Heavy promoter, or a Thy-1 cell surface antigen promoter. In an example, the retinal ganglion cell promoter includes a gamma-Synuclein promoter. In another example, the vector includes a viral vector. In yet another example, the CREB is constitutively active. In still another example, the CREB includes VP-16 CREB.

### BRIEF DESCRIPTION OF THE DRAWINGS

These and other features, aspects, and advantages of the present disclosure will become better understood when the following detailed description is read with reference to the accompanying drawings, wherein:

FIGS. 1A-1Y disclose examples of excitotoxic and optic nerve injury leading to loss of CaMKII activity in RGCs, in accordance with aspects of the present disclosure. (A-F) Confocal images of retinal whole-mounts showing CaMKII phosphorylation (CaMKIIα at T286+CaMKIIβ at T287) in Tuj1-labeled RGCs at 2 hours after PBS (A-C) or NMDA (D-F) injection. Arrowheads, Tuj1⁺ RGCs maintaining (A-C) or losing (D-F) CaMKII activity. Scale bar, 20 μm. (G-H) Quantification of CaMKII phosphorylation in RGCs after excitotoxic injury. (G) The number of total Tuj1+RGCs and pCaMKII+/Tuj1+RGCs 2 hours after PBS control or NMDA injection. Data are presented as mean±s.d., n=5 retinas per group. (H) Percentage of pCaMKII+/Tuj1+RGCs 2 hours after PBS control or NMDA injection. Data are presented as mean±s.d., n=5 retinas per group. Unpaired t-test, *P<0.0001. (I-T) Confocal images of retinal whole-mounts showing CaMKII phosphorylation (CaMKIIα at T286+CaMKIIβ at T287) in Tuj1-labeled RGCs, without injury (I-K) or 5 days (L-N), 7 days (O-Q), and 9 days (R-T) after optic nerve crush (dpc). Arrowheads, Tuj l RGCs losing CaMKII activity (L-T). Scale bar, 20 μm. (U-V) Quantification of CaMKII phosphorylation in RGCs after optic nerve injury. (U) The number of total Tuj1+RGCs and pCaMKII+/Tuj1+RGCs in uninjured retinas and retinas 5 days, 7 days, and 9 days after crush. Data are presented as mean±s.d., n=6 retinas per group. (V) Percentage of pCaMKII+/Tuj1+RGCs in uninjured retinas and retinas 5 days, 7 days, and 9 days after crush. Data are presented as mean±s.d., n=6 retinas per group. One-way ANOVA with Tukey's multiple comparisons test, F:36.22, R²:0.8445, *P<0.0001. (W-X) Confocal images of retinal whole-mounts showing surviving RGCs labeled by Tuj1 immunoreactivity at 7 days after daily injection of PBS (W) or AIP (X). Scale bar, 40 μm. (Y) Quantification of RGC survival, expressed as numbers of RGCs (left Y-axis), and percentages of RGCs relative to that in the uninjured retina (right Y-axis). Data are presented as mean±s.d., n=5 retinas per group. Unpaired t-test, *P<0.0001.

FIGS. 2A-2H disclose examples of excitotoxic and optic nerve injuries leading to loss of CaMKII activity in RGCs, in accordance with aspects of the present disclosure. (A-F) Confocal images of retinal whole-mounts showing pCaMKII immunoreactivity in Tuj1 labeled RGCs without (B) or with (E) blocking peptide phosphorylated at Thr286 for CaMKIIα, (Thr287 for CaMKIIβ). Scale bar, 20 μm. (G) Western blot showing pCaMKII and GAPDH in purified RGCs from uninjured and injured retinas 2 hours after NMDA damage. (H) Relative pCaMKII levels in purified RGCs from uninjured and injured retinas 2 hours NMDA damage. Data are presented as mean±s.d., n=3 blots. Unpaired t-test, *P<0.0001.

FIGS. 3A-3T disclose examples of reactivation of CaMKII protecting RGCs from excitotoxic and optic nerve injuries, in accordance with aspects of the present disclosure. (A-D) Confocal images of retinal whole-mounts showing surviving RGCs labeled by Tuj1 immunoreactivity at 7 days after NMDA injection in control (AAV-EBFP), or AAV-CaMKIIα WT, AAV-CaMKIIα K42R, and AAV-CaMKIIα T286D treated eyes. Scale bar, 40 μm. (E) Quantification of RGC survival after treatment with CaMKIIα variants at 7 days post NMDA injection, expressed as numbers of RGCs (left Y-axis), and percentages of RGCs relative to those in the uninjured retina (right Y-axis). Data are presented as mean±s.d., n=5 retinas per group. One-way ANOVA with Tukey's multiple comparisons test, F:515.5, R²:0.9898, *P<0.0001. (F-I) Confocal images of retinal whole-mounts showing surviving RGCs labeled by Tuj1 immunoreactivity at 7 days after NMDA injection in control (AAV-EBFP), or AAV-CaMKIIβ WT, AAV-CaMKIIβ K43R, and AAV-CaMKIIβ T287D treated eyes. Scale bar, 40 μm. (J) Quantification of RGC survival after treatment with CaMKIIβ variants at 7 days post NMDA injection, expressed as numbers of RGCs (left Y-axis), and percentages of RGCs relative to those in the uninjured retina (right Y-axis). Data are presented as mean±s.d., n=5 retinas per group. One-way ANOVA with Tukey's multiple comparisons test, F:423.3, R²:0.9876, *P<0.0001. (K-N) Confocal images of retinal whole-mounts showing surviving RGCs labeled by Tuj1 immunoreactivity at 2 weeks after optic nerve crush in control (AAV-EBFP), or AAV-CaMKIIα WT, AAV-CaMKIIα K42R, and AAV-CaMKIIα T286D treated eyes. Scale bar, 40 μm. (O) Quantification of RGC survival after treatment with CaMKIIα variants at 2 weeks post optic nerve crush, expressed as numbers of RGCs (left Y-axis), and percentages of RGCs relative to those in the uninjured retina (right Y-axis). Data are presented as mean±s.d., n=5 retinas per group. One-way ANOVA with Tukey's multiple comparisons test, F:379.0, R²:0.9861, *P<0.0001. (P-S) Confocal images of retinal whole-mounts showing surviving RGCs labeled by Tuj1 immunoreactivity at 2 weeks after optic nerve crush in control (AAV-EBFP), or AAV-CaMKIIβ WT, AAV-CaMKIIβ K43R, and AAV-CaMKIIβ T287D treated eyes. Scale bar, 40 μm. (T) Quantification of RGC survival after treatment with CaMKIIβ variants at 2 weeks post optic nerve crush, expressed as numbers of RGCs (left Y-axis), and percentages of RGCs relative to those in the uninjured retina (right Y-axis). Data are presented as mean±s.d., n=5 retinas per group. One-way ANOVA with Tukey's multiple comparisons test, F:361.3, R²:0.9855, *P<0.0001.

FIGS. 4A-4K disclose examples of AAV-mediated gene transfer in RGCs, in accordance with aspects of the present disclosure. (A-C) Confocal images of retinal whole-mounts showing GFP expression in Tuj1+RGCs two weeks after intravitreal injection of AAV-GFP. Scale bar, 40 μm. (D) Transduction efficiency is expressed as a percentage of GFP+RGCs in total RGCs. Data are presented as mean±s.d., n=5 retinas. (E-J) Confocal images of retinal whole-mounts showing pan-CaMKII levels in RGCs two weeks after injection in control (AAV-EBFP) or AAV-CaMKIIα T286D treated eyes. Scale bar, 20 μm. (K) Quantification of pan-CaMKII intensity in RGCs. Data are presented as mean±s.d., n=3 retinas per group. Unpaired t-test,*P=0.0033.

FIGS. 5A-50 disclose examples of performance of more CaMKII variants as well as the RGC-specific promoter mSncg in RGC protections, in accordance with aspects of the present disclosure. (A-E) Confocal images of retinal whole-mounts showing surviving RGCs labeled by Tuj1 immunoreactivity at 7 days after NMDA injection in control (AAV-EBFP), or AAV-CaMKIIα K42D, AAV-CaMKIIα T286A, AAV-CaMKIIα T286D/T305A/T306A, and CaMKIIα T286D/T305D/T306D treated eyes. Scale bar, 40 μm. (F) Quantification of RGC survival after treatment with CaMKIIα variants at 7 days post NMDA injection, expressed as numbers of RGCs (left Y-axis), and percentages of RGCs relative to those in the uninjured retina (right Y-axis). Data are presented as mean±s.d., n=5 retinas per group. One-way ANOVA with Tukey's multiple comparisons test, F:761.4, R²:0.9935, *P=0.0001. (G-H) Confocal images of retinal whole-mounts showing surviving RGCs labeled by Tuj1 immunoreactivity at 7 days post NMDA injection in control (AAV-mSncg-EBFP) or AAV-mSncg-CaMKIIα T286D treated eyes. Scale bar, 40 μm. (I) Quantification of RGC survival at 7 days post NMDA injection, expressed as numbers of RGCs (left Y-axis), and percentages of RGCs relative to those in the uninjured retina (right Y-axis). Data are presented as mean±s.d., n=4 retinas per group. Unpaired t-test, *P<0.0001. (J-N) Confocal images of retinal whole-mounts showing surviving RGCs labeled by Tuj1 immunoreactivity at 7 days after NMDA injection in control (AAV-EBFP), or AAV-CaMKIIβ K43D, AAV-CaMKIIβ T287A, CaMKIIβ T287D/T306A/T307A, and CaMKIIβ T287D/T306D/T307D treated eyes. Scale bar, 40 μm. (0) Quantification of RGC survival after treatment with CaMKIIβ variants at 7 days post NMDA injection, expressed as numbers of RGCs (left Y-axis), and percentages of RGCs relative to those in the uninjured retina (right Y-axis). Data are presented as mean±s.d., n=5 retinas per group. One-way ANOVA with Tukey's multiple comparisons test, F:579.0, R²:0.9914, *P=0.0002.

FIGS. 6A-6O disclose examples of reactivation of CaMKII providing post-injury and long-term RGC protection after excitotoxic or axonal injuries, in accordance with aspects of the present disclosure. (A-B) Confocal images of retinal whole-mounts showing surviving RGCs labeled by Tuj1 immunoreactivity at 2 weeks after optic nerve crush in control (AAV-EBFP) or AAV-CaMKIIα T286D post-injury treatment. Scale bar, 40 μm. (C) Quantification of RGC survival 2 weeks after optic nerve crush, expressed as numbers of RGCs (left Y-axis), and percentages of RGCs relative to those in the uninjured retina (right Y-axis). Data are presented as mean±s.d., n=5 retinas per group. Unpaired t-test, *P<0.0001. (D-G) Confocal images of retinal whole-mounts showing surviving RGCs labeled by Tuj1 immunoreactivity at 2 months and 12 months post NMDA injection in control (AAV-EBFP) and AAV-CaMKIIβ T287D treated eyes. Scale bar, 40 μm. (H) Quantification of RGC survival 2 months and 12 months post NMDA injection, expressed as numbers of RGCs (left Y-axis), and percentages of RGCs relative to those in the uninjured retina (right Y-axis). Data are presented as mean±s.d., n=4 retinas per group. One-way ANOVA with Tukey's multiple comparisons test, F:1370, R²:0.9971, *P<0.0001. (I-N) Confocal images of retinal whole-mounts showing surviving RGCs labeled by Tuj1 immunoreactivity at 1 month, 2 months, and 6 months post optic nerve crush in control (AAV-EBFP) or AAV-CaMKIIβ T287D treated eyes. Scale bar, 40 μm. (0) Quantification of RGC survival 1 month, 2 months and 6 months post optic nerve injury, expressed as numbers of RGCs (left Y-axis), and percentages of RGCs relative to those in the uninjured retina (right Y-axis). Data are presented as mean±s.d., n=4 retinas per group. One-way ANOVA with Tukey's multiple comparisons test, F:523.2, R²:0.9932, *P<0.0001.

FIGS. 7A-7Z disclose examples of CREB acting downstream of CaMKII to protect RGCs from excitotoxic and optic nerve injuries, in accordance with aspects of the present disclosure. (A-C) Confocal images of retinal whole-mounts showing CREB phosphorylation in RGCs, from uninjured eyes (A), and 2 hours after NMDA injection in control (AAV-EBFP) (B) or AAV-CaMKIIα T286D (C) treated eyes. Arrowheads, Tuj1⁺ RGCs maintaining (A) or losing (B) CREB activity, which was restored after treatment with CaMKIIα T286D (C). Scale bar, 20 μm. (D-E) Quantification of CREB phosphorylation in RGCs 2 hours after NMDA-induced excitotoxic injury. (D) The number of total Tuj1+RGCs and pCREB+/Tuj1+RGCs in uninjured or NMDA-damaged eyes. Data are presented as mean±s.d., n=6 retinas per group. (E) Percentage of pCREB+/Tuj1+RGCs in uninjured or NMDA damaged eyes. Data are presented as mean±s.d., n=6 retinas per group. One-way ANOVA with Tukey's multiple comparisons test, F:6139, R²:0.9988, *P<0.0001. (F-G) Confocal images of retinal whole-mounts showing surviving RGCs labeled by Tuj1 immunoreactivity at 7 days after NMDA injection in AAV-CaMKIIα T286D+ Control (AAV-EBFP), or AAV-CaMKIIα T286D+AAV-A-CREB treated eyes. Scale bar, 40 μm. (H) Quantification of RGC survival, expressed as numbers of RGCs (left Y-axis), and percentages of RGCs relative to those in the uninjured retina (right Y-axis). Data are presented as mean±s.d., n=5 retinas per group. Unpaired t-test, *P<0.0001. (I-J) Confocal images of retinal whole-mounts showing surviving RGCs labeled by Tuj1 immunoreactivity at 7 days after NMDA injection in control (AAV-EBFP) or AAV-VP16-CREB treated eyes. Scale bar, 40 μm. (K) Quantification of RGC survival, expressed as numbers of RGCs (left Y-axis), and percentages of RGCs relative to those in the uninjured retina (right Y-axis). Data are presented as mean±s.d., n=5 retinas per group. Unpaired t-test, *P<0.0001. (L-R) Confocal images of retinal whole-mounts showing CREB phosphorylation in RGCs, from uninjured eyes (L), and 5 days, 7 days and 9 days after optic nerve crush in control (AAV-EBFP) (M-O) or AAV-CaMKIIα T286D (P-R) treated eyes. Arrowheads, Tuj1⁺ RGCs losing CREB activity (M-O). Scale bar, 20 μm. (S-T) Quantification of CREB phosphorylation in RGCs after optic nerve injury. (S) The number of total Tuj1+RGCs and pCREB+/Tuj1+RGCs in uninjured and injured retinas 5 days, 7 days, and 9 days after crush. Data are presented as mean±s.d., n=6 retinas per group. (T) Percentage of pCREB+/Tuj1+RGCs in uninjured and injured retinas 5 days, 7 days, and 9 days after crush. Data are presented as mean±s.d., n=6 retinas per group. One-way ANOVA with Tukey's multiple comparisons test, F:89.58, R²:0.9389, *P<0.0001. (U-V) Confocal images of retinal whole-mounts showing surviving RGCs labeled by Tuj1 immunoreactivity at 2 weeks after optic nerve crush in AAV-CaMKIIα T286D+ Control (AAV-EBFP), or AAV-CaMKIIα T286D+AAV-A-CREB treated eyes. Scale bar, 40 μm. (W) Quantification of RGC survival, expressed as numbers of RGCs (left Y-axis), and percentages of RGCs relative to those in the uninjured retina (right Y-axis). Data are presented as mean±s.d., n=5 retinas per group. Unpaired t-test, *P<0.0001. (X-Y) Confocal images of retinal whole-mounts showing surviving RGCs labeled by Tuj1 immunoreactivity at 2 weeks after optic nerve crush in control (AAV-EBFP) or AAV-VP16-CREB treated eyes. Scale bar, 40 μm. (Z) Quantification of RGC survival, expressed as numbers of RGCs (left Y-axis), and percentages of RGCs relative to those in the uninjured retina (right Y-axis). Data are presented as mean±s.d., n=5 retinas per group. Unpaired t-test, *P<0.0001.

FIGS. 8A-8V disclose examples of signaling mechanisms downstream of CaMKII in RGC protections, in accordance with aspects of the present disclosure. (A-B) Confocal images of retinal whole-mounts showing CREB phosphorylation in RGCs 2 hours after NMDA injection in AAV-CaMKIIα T286D+ control (AAV-EBFP), or AAV-CaMKIIα T286D+AAV-A-CREB treated eyes. Arrowheads, Tuj1⁺ RGCs losing CREB activity. Scale bar, 20 μm. (C-D) Quantification of CREB phosphorylation in RGCs after excitotoxic injury. (C) The number of total Tuj1+RGCs and pCREB+/Tuj1+RGCs 2 hours after NMDA injection. Data are presented as mean±s.d., n=4 retinas per group. (D) Percentage of pCREB+/Tuj1+RGCs 2 hours NMDA injection. Data are presented as mean±s.d., n=4 retinas per group. Unpaired t-test, *P<0.0001. (E-F) Confocal images of retinal whole-mounts showing CREB phosphorylation in RGCs 2 hours after NMDA injection in control (AAV-EBFP) or AAV-VP16-CREB treated eyes. Arrowheads, Tuj1⁺ RGCs losing CREB activity. Scale bar, 20 μm. (G-H) Quantification of CREB phosphorylation in RGCs after excitotoxic injury. (G) The number of total Tuj1+RGCs and pCREB+/Tuj1+RGCs 2 hours after NMDA injection. Data are presented as mean±s.d., n=3 retinas per group. (H) Percentage of pCREB+/Tuj1+RGCs 2 hours NMDA injection. Data are presented as mean±s.d., n=3 retinas per group. Unpaired t-test, *P=0.0003. (I) Confocal images of retinal whole-mounts showing TrkB phosphorylation in RGCs, from uninjured eyes, and 2 hours after NMDA injection in control (AAV-EBFP) or AAV-CaMKIIα T286D treated eyes. Scale bar, 20 μm. (J) Quantification of pTrkB intensity in RGCs. Data are presented as mean±s.d., n=3 retinas per group. One-way ANOVA with Tukey's multiple comparisons test, F:19.26, R²:0.8652, *P=0.0124. (K-L) Confocal images of retinal whole-mounts showing CREB phosphorylation in RGCs 5 days after optic nerve crush in AAV-CaMKIIα T286D+ control (AAV-EBFP), or AAV-CaMKIIα T286D+AAV-A-CREB treated eyes. Arrowheads, Tuj l RGCs losing CREB activity. Scale bar, 20 μm. (M-N) Quantification of CREB phosphorylation in RGCs 5 days after nerve injury. (M) The number of total Tuj1+RGCs and pCREB+/Tuj1+RGCs 5 days after optic nerve crush. Data are presented as mean±s.d., n=4 retinas per group. (N) Percentage of pCREB+/Tuj1+RGCs 5 days after optic nerve crush. Data are presented as mean±s.d., n=4 retinas per group. Unpaired t-test, *P<0.0001. (O-P) Confocal images of retinal whole-mounts showing CREB phosphorylation in RGCs 5 days after optic nerve crush in control (AAV-EBFP) or AAV-VP16-CREB treated eyes. Arrowheads, Tuj1⁺ RGCs losing CREB activity. Scale bar, 20 μm. (Q-R) Quantification of CREB phosphorylation in RGCs 5 days after nerve injury. (Q) The number of total Tuj1+RGCs and pCREB+/Tuj1+RGCs 5 days after optic nerve crush. Data are presented as mean±s.d., n=3 retinas per group. (R) Percentage of pCREB+/Tuj1+RGCs 5 days after optic nerve crush. Data are presented as mean±s.d., n=3 retinas per group. Unpaired t-test, *P=0.0002. (S) Confocal images of retinal whole-mounts showing DLK staining in RGCs, from uninjured eyes, and 3 days after optic nerve crush in control (AAV-EBFP) or AAV-CaMKIIα T286D treated eyes. Scale bar, 20 μm. (T) Quantification of DLK intensity in RGCs. Data are presented as mean±s.d., n=3 retinas per group. One-way ANOVA with Tukey's multiple comparisons test, F:192.3, R²:0.9846, n.s. (not significant, P=0.18). (U) Confocal images of retinal whole-mounts showing c-Jun phosphorylation in RGCs, from uninjured eyes, and 3 days after optic nerve crush in control (AAV-EBFP) or AAV-CaMKIIα T286D treated eyes. Scale bar, 20 μm. (V) Quantification of p-c-Jun intensity in RGCs. Data are presented as mean±s.d., n=3 retinas per group. One-way ANOVA with Tukey's multiple comparisons test, F:87.73, R²:0.9669, n.s. (not significant, P=0.09).

FIGS. 9A-9I disclose examples of CaMKII-mediated protection of RGCs in induced and genetic models of glaucoma, in accordance with aspects of the present disclosure. (A) Image of magnetic microbeads distributed evenly around the circumference of the anterior chamber using magnets after injection. (B) Image of the eye section after H&E staining shows microbeads accumulation at the iridocorneal angle. Scale bar, 100 μm. (C) Quantification of intraocular pressure (IOP) after injection of PBS (sham) or microbeads. Data are presented as mean±s.d., n=6 eyes per group. (D-E) Confocal images of retinal whole-mounts showing surviving RGCs labeled by Tuj1 immunoreactivity at 2 months after induction of elevated IOP in Control (AAV-EBFP) or AAV-CaMKIIα T286D treated eyes. Scale bar, 40 μm. (F) Quantification of RGC survival, expressed as numbers of RGCs (left Y-axis), and percentages of RGCs relative to those in the uninjured retina (right Y-axis). Data are presented as mean±s.d., n=6 retinas per group. Unpaired t-test, *P<0.0001. (G-H) Confocal images of retinal whole-mounts from 2-month-old GLAST−/− mice showing surviving RGCs labeled by Tuj1 immunoreactivity in Control (AAV-EBFP) or AAV-CaMKIIα T286D treated eyes. Scale bar, 40 μm. (I) Quantification of RGC survival in GLAST−/− retinas, expressed as numbers of RGCs (left Y-axis), and percentages of RGCs relative to those in the uninjured wild-type retina (right Y-axis). Data are presented as mean±s.d., n=5 retinas per group. Unpaired t-test, *P<0.0001.

FIGS. 10A-10P disclose examples of CaMKII-mediated protection of RGC axons in induced and genetic models of glaucoma, in accordance with aspects of the present disclosure. (A-D) Confocal images of retinal whole-mounts showing pan-CaMKII levels in RGCs of uninjured retinas or 2 weeks after microbeads injection in AAV-CaMKIIα T286D treated retinas. Scale bar, 20 μm. (E) Quantification of pan-CaMKII intensity in RGCs. Data are presented as mean±s.d., n=3 retinas per group. Unpaired t-test, *P<0.0001. (F-H) Light microscope images of semithin sections of optic nerve stained with toluidine blue, from uninjured eyes, and at 2 months after induction of elevated IOP in Control (AAV-EBFP) or AAV-CaMKIIα T286D treated eyes. Scale bar, 4 μm. (I) Quantification of axon survival, expressed as numbers of axons (left Y-axis), and percentages of axons relative to those in the uninjured eyes (right Y-axis). Data are presented as mean±s.d., n=4 nerves per group. Unpaired t-test, *P=0.0056. (J-M) Confocal images of retinal whole-mounts showing pan-CaMKII levels in RGCs of uninjured retinas or AAV-CaMKIIα T286D treated retinas of GLAST−/− mice at 3 weeks after AAV injection. Scale bar, 20 μm. (N) Quantification of pan-CaMKII intensity in RGCs. Data are presented as mean±s.d., n=3 retinas per group. Unpaired t-test, *P=0.0055. (O-P) Images of sections from 8-month-old GLAST−/− mice showing optic nerve head morphology (bright light) and nuclear layers stained with DAPI (blue) in Control (AAV-EBFP) or AAV-CaMKIIα T286D treated eyes. Scale bar, 80 μm.

FIGS. 11A-11M disclose examples of CaMKII reactivation protecting RGC axons and their target projections to the brain, in accordance with aspects of the present disclosure. (A) Schematic illustration of anterograde Cholera Toxin Subunit B (CTB) tracing of the optic nerve, lateral geniculate nucleus (LGN), and superior colliculus (SC). (B-D) Confocal images of anterograde CTB tracing of RGC axons in the optic nerve, from uninjured eyes, and 7 days after NMDA injection in control (PBS) or AAV-CaMKIIα T286D treated eyes. Scale bar, 300 μm. Inserts: whole-mount retinal images showing CTB filling in the retina. (E) Quantification of CTB intensity in the optic nerve. Data are presented as mean±s.d., n=4 nerves per group. One-way ANOVA with Tukey's multiple comparisons test, F:281.7, R²:0.9843, *P<0.0001. (F-H) Confocal images of anterograde CTB tracing of RGC axons projecting to the contralateral LGN from uninjured eyes, and 7 days after NMDA injection in control (PBS) or AAV-CaMKIIα T286D treated eyes. Scale bar, 300 μm. (I) Quantification of CTB intensity in the contralateral LGN. Data are presented as mean±s.d., n=4 brains per group. One-way ANOVA with Tukey's multiple comparisons test, F:155.8, R²:0.9719, *P<0.0001. (J-L) Confocal images of anterograde CTB tracing of RGC axons projecting to the contralateral SC, from uninjured eyes, and 7 days after NMDA injection in control (PBS) or AAV-CaMKIIα T286D treated eyes. Scale bar, 300 μm. (M) Quantification of CTB intensity in the contralateral LGN. Data are presented as mean±s.d., n=4 brains per group. One-way ANOVA with Tukey's multiple comparisons test, F:226.9, R²:0.9805, *P<0.0001.

FIGS. 12A-12H disclose examples of CaMKII reactivation protecting RGC axonal projections to the ipsilateral hemisphere, in accordance with aspects of the present disclosure. (A-C) Confocal images of anterograde CTB tracing of RGC axons projecting to the ipsilateral LGN, from uninjured eyes, and 7 days after NMDA injection in control (PBS) or AAV-CaMKIIα T286D treated eyes. Scale bar, 300 μm. (D) Quantification of CTB intensity in the ipsilateral LGN. Data are presented as mean±s.d., n=4 brains per group. One-way ANOVA with Tukey's multiple comparisons test, F:145.0, R²:0.9699, *P<0.0001. (E-G) Confocal images of anterograde CTB tracing of RGC axons projecting to the ipsilateral SC from uninjured eyes, and 7 days after NMDA injection in control (PBS) or AAV-CaMKIIα T286D treated eyes. Scale bar, 300 μm. (H) Quantification of CTB intensity in the ipsilateral SC. Data are presented as mean±s.d., n=4 brains per group. One-way ANOVA with Tukey's multiple comparisons test, F:162.2, R²:0.9730, *P<0.0001.

FIGS. 13A-13Q disclose examples of CaMKII reactivation preserving functional vision, in accordance with aspects of the present disclosure. (A-C) Representative responses of PERG recordings, from uninjured eyes, and 7 days after NMDA injection, in control (PBS) or AAV-CaMKIIα T286D treated eyes. (D) Quantification of PERG amplitudes. Data are presented as mean±s.d., n=4 mice per group. One-way ANOVA with Tukey's multiple comparisons test, F:47.95, R²:0.9142, *P<0.0001. (E-G) Representative responses of PVEP recordings from uninjured eyes, and 10 days after NMDA injection, in control (PBS) or AAV-CaMKIIα T286D treated eyes. (H) Quantification of PVEP amplitudes. PVEP amplitudes are shown for each animal (red) and averaged across the group (blue). Data are presented as mean±s.d., n=4 mice per group. One-way ANOVA with Tukey's multiple comparisons test, F:40.67, R²:0.9004,*P=0.0002. (I) Schematic diagram of the visual water task. (J-L) Visual water task performance as a function of spatial frequencies, from uninjured mice, and 4-14 days after NMDA injection, in control (PBS) or AAV-CaMKIIα T286D treated (both eyes) mice. For each column, each row shows the results from a single mouse. For each animal, a trendline of best fit was generated, and the point on the curve that intersected with 70% correct choices was adopted as the threshold for acuity. (M) Acuity (Spatial frequency thresholds) measured in the visual water task. Data are presented as mean±s.d., n=4 mice per group. One-way ANOVA with Tukey's multiple comparisons test, F:529.8, R²:0.9916, *P<0.0001. (N) Schematic diagram of the visual cliff test. (O) Visual cliff performance, from uninjured mice, and 7 days after NMDA injection in control (PBS) or AAV-CaMKIIα T286D treated mice. Data show the number (left Y-axis) and percentage (right Y-axis) of shallow/deep side choices. Fisher's exact test, *P=0.0373. (P) Schematic diagram of the looming response test. (Q) Performance in response to looming stimuli, from uninjured mice, and 7 days after NMDA injection in control (PBS) or AAV-CaMKIIα T286D treated mice. Data show the number (left Y-axis) and percentage (right Y-axis) of responders and non-responders. Fisher's exact test, *P=0.0028.

### DETAILED DESCRIPTION

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

This disclosure relates to a method of decreasing degeneration of retinal ganglion cells in a subject, a method of treating vision loss in a subject, and a pharmaceutical composition. In an example, the pharmaceutical composition includes one or more components applicable for use in the methods disclosed herein.

CaMK and CREB signaling are disclosed herein to be severely compromised after excitotoxic injury to RGC somas or optic nerve injury to RGC axons, and increasing activity of these pathways are disclosed herein to robustly protect RGCs from injury. CaMK is disclosed herein to protect RGCs in induced and genetic models of glaucoma, a leading cause of blindness characterized by loss of RGC somas and axons. Also disclosed herein is that increasing activity of CaMK protects long distance RGC axon projections and restores visual function in the entire visual pathway from the retina to primary visual cortex in the brain. Also disclosed herein is that increasing activity of CREB protects RGCs.

The present disclosure of a protective effect increasing CaMK activity has on RGC and vision is particularly surprising, in view of prior evidence that inhibiting CaMK blunts cytotoxicity. Calcium is a highly versatile intracellular signal responsible for regulating an array of cellular processes (Berridge et al., 2000). Loss of Ca2+ homeostasis, often in the form of cytoplasmic increases, leads to cell injuries (Dong et al., 2006). Aberrant Ca2+ activation is known to be involved in RGC death following insults such as excitotoxicity and optic nerve injury (Hartwick et al., 2008; Prilloff et al., 2007). CaMKII is a central coordinator and executor of Ca2+ signal transduction (Hudmon and Schulman, 2002a). Accordingly, previous studies have shown that CaMKII inhibition protects RGCs from excitotoxic cell death, suggesting that increased CaMK activity promotes RGC degeneration. On the contrary, as surprisingly disclosed herein, promoting CaMK activity is protective for RGC and vision.

As disclosed herein, excitotoxic insults to RGC somas or optic nerve injury to RGC axons led to inactivation of CaMK and its downstream target CREB in RGCs. Increasing activity of CaMK or CREB protected RGCs from both injuries. Furthermore, CaMK-mediated RGC protection slowed down the disease progression in induced and genetic animal models of glaucoma. Increasing CaMK activity not only saves RGC somas, but also protects long distance RGC axon projections from the retina to visual relay centers in the brain. Increasing CaMK mediated protection of RGCs also restores functional vision in the entire visual pathway, evidenced by improved visual responses in the retina and the primary visual cortex in the brain as well as visually-guided behavior. Also disclosed is targeting increased activity of CaMK or CREB as methods of decreasing degeneration of RGCs and of treating vision loss, and pharmaceutical compositions including compositions for increasing CaMK activity or CREB activity, including in RGC.

Isoforms of CaMK include CamKI, CaMKII, and CaMKIV. As disclosed herein, increasing activity of any of these CaMKs prevents RGC degeneration. For example, increasing activity of CaMKI, CaMKII, or CaMKIV prevents RGC degeneration. CaMKII includes several isoforms, including CaMKIIα, CaMKIIβ, CaMKIIγ, and CaMKIIδ. Increasing activity of CaMKIIα or CaMKIIβ prevents RGC degeneration. Increasing CaMKII activity also improves vision following insults known to impair RGC compared to subjects exposed to such insults with exposure to a treatment to increase CaMK activity. Given the known shared cellular functionalities among CaMKs, including CaMKI, CaMKII (including without limitation CaMKIIα, CaMKIIβ, CaMKIIγ, and CaMKIIδ), and CaMKIV, and ability of various CaMKs to prevent RGC degeneration and to treat vision loss as disclosed herein, a skilled person would apprehend that increasing activity of any one or more of CaMKI, CaMKII (including without limitation CaMKIIα, CaMKIIβ, CaMKIIγ, and CaMKIIδ), and CaMKIV may prevent RGC degeneration, may prevent RGC somata loss, may prevent loss of RGC axon projections in the brain, may prevent RGC axonal loss, may prevent vision loss, may treat vision loss, and any one or more of the foregoing.

In some examples, increasing activity of a CaMK may include increasing activity of a variant of a CaMK with an amino acid sequence that differs from a CaMK expressed by the subject, or expressed by the subject in a cell or cells in which activity of the CaMK is increased in accordance with the methods disclosed herein. For example, increasing activity of a CaMK may include increasing activity of a CaMK that differs from a CaMK as disclosed in the present disclosure, or from a CaMK encoded by the subject's genome, or from a CaMK that would otherwise be expressed in the subject's cell or cells in which CaMK activity is increased in accordance with the methods disclosed herein, by about 1% or more, or by about 2% or more, or by about 3% or more, or by about 4% or more, or by about 5% or more, or by about 6% or more, or by about 7% or more, or by about 8% or more, or by about 9% or more, or by about 10% or more, or by about 11% or more, or by about 12% or more, or by about 13% or more, or by about 14% or more, or by about 15% or more, or by about 16% or more, or by about 17% or more, or by about 18% or more, or by about 19% or more, or by about 20% or more, or by about 21% or more, or by about 22% or more, or by about 23% or more, or by about 24% or more, or by about 25% or more, or by about 30% or more, or by about 35% or more.

In other examples, increasing activity of a CaMK may include increasing activity of a CaMK that differs from a CaMK as disclosed in the present disclosure, or from a CaMK encoded by the subject's genome, or from a CaMK that would otherwise be expressed in the subject's cell or cells in which CaMK activity is increased in accordance with the methods disclosed herein by including one or more amino acid substitution, insertion, or deletion of about 1 or more, about 2 or more, about 3 or more, about 4 or more, about 5 or more, about 10 or more, about 15 or more, about 20 or more, about 25 or more, about 30 or more, about 25 or more, about 40 or more, or about 50 or more amino acids relative to a foregoing CaMK, alone or in combination.

In other examples, increasing activity of a CaMK may include increasing activity of a CaMK that is constitutively active. By constitutively active is meant a CaMK whose activity, or increased activity, or sustained activity, is not dependent on or diminished by one or more other cell signaling events otherwise or generally required to increase or capable of decreasing activity of a CaMK in the subject or in the subject's cell or cells in which CaMK activity is increased in accordance with the methods disclosed herein. As a nonlimiting example, activation of CaMKII may generally be initiated by Ca2+ influx and subsequent Ca2+/Calmodulin binding; the resultant conformation change of CaMKII allows its autophosphorylation at either Threonine 286 (T286) for CaMKIIα or Threonine 287 (T287) for CaMKIIβ, which may enhance activity of such isoform of both isoforms, whereby if autophosphorylation occurs, CaMK may remain active after Ca2+ concentration falls. In another example, a constitutively active CaMK may include a truncated, N-terminal catalytic domain of CaMKIIα, or a truncated, N-terminal catalytic domain of CaMKIIβ, which truncations are constitutively active.

In an example, increasing activity of CaMK may include increasing activity of a constitutively active variant of CaMKIIα or of CaMKIIβ, or of another CaMK, such as any of the foregoing variants, without limitation. As a non-limiting example, increasing activity of a CaMK in accordance with the present disclosure may include increasing levels, expression, or activity of a T286D substituted CaMKIIα, which, without being limited to any particular mechanism of action, may simulate an active, phosphorylated state of CaMK. In another non-limiting example, increasing levels, expression, or activity of a CaMK in accordance with the present disclosure may include increasing activity of a T287D substituted CaMKIIβ, which, without being limited to any particular mechanism of action, may simulate an active, phosphorylated state of CaMK. In another example, increasing activity of a CaMK may include increasing levels, expression, or activity of an N-terminal catalytic domain of CaMKIIα, or an N-terminal catalytic domain of CaMKIIβ, which are known to be constitutively active.

In some examples, increasing activity of CREB may include increasing activity of a variant of CREB with an amino acid sequence that differs from a CREB expressed by the subject, or expressed by the subject in a cell or cells in which activity of the CREB is increased in accordance with the methods disclosed herein. For example, increasing activity of a CREB may include increasing activity of a CREB that differs from a CREB as disclosed in the present disclosure, or from a CREB encoded by the subject's genome, or from a CREB that would otherwise be expressed in the subject's cell or cells in which CREB activity is increased in accordance with the methods disclosed herein, by about 1% or more, or by about 2% or more, or by about 3% or more, or by about 4% or more, or by about 5% or more, or by about 6% or more, or by about 7% or more, or by about 8% or more, or by about 9% or more, or by about 10% or more, or by about 11% or more, or by about 12% or more, or by about 13% or more, or by about 14% or more, or by about 15% or more, or by about 16% or more, or by about 17% or more, or by about 18% or more, or by about 19% or more, or by about 20% or more, or by about 21% or more, or by about 22% or more, or by about 23% or more, or by about 24% or more, or by about 25% or more, or by about 30% or more, or by about 35% or more.

In other examples, increasing activity of a CREB may include increasing activity of a CREB that differs from a CREB as disclosed in the present disclosure, or from a CREB encoded by the subject's genome, or from a CREB that would otherwise be expressed in the subject's cell or cells in which CREB activity is increased in accordance with the methods disclosed herein by including one or more amino acid substitution, insertion, or deletion of about 1 or more, about 2 or more, about 3 or more, about 4 or more, about 5 or more, about 10 or more, about 15 or more, about 20 or more, about 25 or more, about 30 or more, about 25 or more, about 40 or more, or about 50 or more amino acids relative to a foregoing CREB, alone or in combination.

In other examples, increasing activity of a CREB may include increasing activity of a CREB that is constitutively active. By constitutively active is meant a CREB whose activity, or increased activity, or sustained activity, is not dependent on or diminished by one or more other cell signaling events otherwise or generally required to increase or capable of decreasing activity of a CREB in the subject or in the subject's cell or cells in which CREB activity is increased in accordance with the methods disclosed herein. As a nonlimiting example, increasing activity of CREB may include increasing expression of a CREB variant known as VP16-CREB, a fusion between the activation domain of herpes simplex virus VP16 protein and the DNA binding domain of CREB, as disclosed in U.S. Pat. No. 9,587,000, incorporated herein in its entirety by reference.

CREB activity may be increased by increasing an amount of CREB in a subject, such as in a subject's retina, including in a subject's RGCs. CREB expression may be increased by transfecting cells such as RGC with a CREB or with a polynucleotide sequence encoding a CREB so as to cause expression of a CREB from the polynucleotide in the subject or cell or cells thereof. In some examples including transfecting a cell with a polynucleotide encoding a CREB, the polynucleotide may further include a cis-regulatory element operatively associated with the portion of the polynucleotide encoding the CREB so as to stimulate, promote, or enhance expression of CREB from the polynucleotide. Such a cis-regulatory element may include one or more of a promoter sequence and an enhancer sequence.

A cis-regulatory element may include a promotor, an enhancer, or both. In some cases, a sequence for a cis-regulatory element may be located within fewer than 10 nucleotides from a transcription start site, fewer than 20 nucleotides from a transcription start site, fewer than 30 nucleotides from a transcription start site, fewer than 40 nucleotides from a transcription start site, fewer than 50 nucleotides from a transcription start site, fewer than 60 nucleotides from a transcription start site, fewer than 70 nucleotides from a transcription start site, fewer than 80 nucleotides from a transcription start site, fewer than 90 nucleotides from a transcription start site, fewer than 100 nucleotides from a transcription start site, fewer than 125 nucleotides from a transcription start site, fewer than 150 nucleotides from a transcription start site, fewer than 175 nucleotides from a transcription start site, fewer than 200 nucleotides from a transcription start site, fewer than 225 nucleotides from a transcription start site, fewer than 250 nucleotides from a transcription start site, fewer than 275 nucleotides from a transcription start site, fewer than 300 nucleotides from a transcription start site, fewer than 325 nucleotides from a transcription start site, fewer than 35 nucleotides from a transcription start site, fewer than 375 nucleotides from a transcription start site, fewer than 400 nucleotides from a transcription start site, fewer than 425 nucleotides from a transcription start site, fewer than 450 nucleotides from a transcription start site, fewer than 475 nucleotides from a transcription start site, fewer than 500 nucleotides from a transcription start site, or between 500 and 1,000 nucleotides from a transcription start site

A promoter is a nucleotide sequence to which RNA polymerizing enzymes bind for initiation of transcription of a downstream gene sequence. Many genes that show tissue- or cell-type specific expression including a promotor upstream of the DNA sequence that codes for the RNA that is particularly active in cells where the gene is expressed. A promoter may be more active in some cells than other, such as being active only in specific ell- or tissue-types, or highly active in certain cell- or tissue-types relative to others. Promoters include a sequence where transcription is initiated. Eukaryotic promoters may and typically do include features such as a TATA box, a transcription factor IIB recognition site, and a core promotor sequence (or an initiator). Transcription factors bind and RNA polymerase bind to a promoter for transcription initiation.

Also included in a cis-regulatory element may be one or more enhancer sequence. An enhancer is part of a cis-regulatory element that enhances transcription initiated in or by the promotor. An enhancer may serve to promote an initiation of transcription at a promoter, for example, such as through binding of additional transcription factors to the enhancer that facilitate or enhance recruitment of other factors and transcriptional machinery to the promotor. As with promotors, many genes have enhances that are involved in cell- or tissue-specific or cell- or tissue-enhanced expression. In some examples, a cis-regulatory element may include other features such as intronic sequences, splice sites, exonic sequences, or any combinations thereof, that may influence transcript expression in a given cellular environment. In an example, a cell-type specific cis-regulatory element may include features that repress expression in cell types other than those in which the cell type-specific cis-regulatory element is intended to drive expression.

In an example, a cis-regulatory element may include a promiscuous cis-regulatory element. A promiscuous cis-regulatory element may include one or more polynucleotide sequence that may, or be designed to, drive expression without, or with minimal, regard to cell type transfected by the polynucleotide. A promiscuous cis-regulatory element may promote expression of a polynucleotide encoding a CaMK or CREB in different cell types, including cells of different tissues, lineages, ages, etc. Examples of promiscuous cis-regulatory elements include a CMV early enhancer/chicken R actin (CAG) promoter cis-regulatory element, a human 3-actin promoter cis-regulatory element, a human elongation factor-la promoter cis-regulatory element, a cytomegalovirus (CMV) promoter cis-regulatory element, a simian virus 40 promoter cis-regulatory element, and herpes simplex virus thymidine kinase In another example, a cis-regulatory element may include a cell-specific cis-regulatory element.

A cell-specific cis-regulatory element may include one or more polynucleotide sequence that may, or be designed to, drive expression only, or mostly, or preferentially, or predominantly, in a predetermined cell type or types. A cell-specific cis-regulatory element may include one or more polynucleotide sequence that may, or be designed to, drive expression only, or mostly, or preferentially, or predominantly, in a predetermined cell type or types without, or with minimal, or negligible, or insubstantial, expression in other cell types that may be transfected with the polynucleotide, or not so as to increase CaMK or CREB activity in such sells or to do so only to a minimal, or negligible, or insubstantial degree relative to activity induced in the cell type or types in which the cis-regulatory element is designed to drive expression.

A cell-specific cis-regulatory element, in accordance with the present disclosure, may increase expression in a transfected cell type in which the cis-regulatory element is intended or designed to drive expression by about 0%, about 5% or less, about 10% or less, 15% or less, about 20% or less, about 25% or less, about 30% or less, about 35% or less, about 40% or less, about 45% or less, about 50% or less, about 55% or less, about 60% or less, about 65% or less, about 70% or less, about 75% or less, about 80% or less, about 85% or less, about 90% or less, about 95% or less, about 100% or less, about 150% or less, about 200% or less, about 250% or less, about 300% or less, about 350% or less, about 400% or less, about 450% or less, about 500% or less, about 550% or less, about 600% or less, about 650% or less, about 700% or less, about 750% or less, about 800% or less, about 850% or less, about 900% or less, about 1,000% or less, or more, compared to a level of expression in a transfected cell type or types other than that in which the cell-specific cis-regulatory element is intended or designed to drive expression. In another example, a cell-specific cis-regulatory element, in accordance with the present disclosure, may cause expression in a transfected cell type in which the cis-regulatory element is intended or designed to drive expression but no, minimal, negligible, or undetectable levels of expression in a transfected cell type or types in which the cell-specific cis-regulatory element is not designed or intended to drive expression.

In an example, the cis-regulatory element may be a cis-regulatory element that drives expression of a transcript in RGC. For example, the cis-regulatory element may be a promoter that drives expression of a transcript in RGC, referred to herein as an RGC promoter. For example, the cis-regulatory element may be a promoter, enhancer, or both, of a transcript known to be expressed in RGC. The cis-regulatory element may be a promoter, enhancer, or both of a transcript known to be expressed in RGC to a higher degree than the transcript is expressed in other cells of the retina or other cells of the eye. In an example, an RGC promoter may be a promoter of a transcript whose expression is higher in RGCs relative to other cells of tissues of the eye, or relative to other cells of the retina. In an example, an RGC promoter may drive a level of expression of a transcript in RGC that is sufficiently higher that a level of expression of the transcript in other cells of tissues of the eye, or of other cells of the retina, sufficient to permit identification of a cell as an RGC on the basis of a the differentiable level of expression of the transcript in the RGC compared to other cells.

In an example, an RGC promoter may not drive expression of a transcript in cells of other tissue of the eye or other retinal cells, other than RGC. For example, a transcript may be detectable in an RGC (such as by in situ hybridization detection of mRNA of the transcript) but, in an example, not be detectable in cells of other tissues of the eye or, in another example, not be detectable in other cell types of the retina. In another example, an RGC promoter may drive expression of a transcript in an RGC that is at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 25, or at least about 50, or at least about 75, or at least about 100, or at least about 150, or at least about 200, or at least about 250, or at least about 300, or at least about 350, or at least about 400, or at least about 450, or at least about 500, or at least about 600, or at least about 700, or at least about 800, or at least about 900, or at least about 1,000, or at least about 5,000, or at least about 10,000, or at least about 25,000, or at least about 50,000, or at least about 75,000, or at least about 100,000 times a level of expression of the transcript in, n an example, a cell of another tissue of the eye or, in another example, another cell type of the retina. A level of expression may be determined by measurement of transgene expression by western blot analysis in purified RGC compared to other cell type, such as in other purified cell type of cell from eye tissue.

In an example, as disclosed herein, an RGC promoter may be a gamma-Synuclein promoter, such as a human gamma-Synuclein promoter, a mouse gamma-Synuclein promoter, or another gamma-Synuclein promoter that drives expression of a transcript in RGC of a subject. In another example, an RGC promoter may be a Synapsin 1 promoter, such as a human Synapsin 1 promoter, a mouse Synapsin 1 promoter, or another Synapsin 1 promoter that drives expression of a transcript in RGC of a subject. In another example, an RGC promoter may be a Thy-1 cell surface antigen promoter, such as a human Thy-1 cell surface antigen promoter, a mouse Thy-1 cell surface antigen promoter, or another Thy-1 cell surface antigen promoter that drives expression of a transcript in RGC of a subject. In another example, an RGC promoter may be a Neurofilament Heavy promoter, such as a human Neurofilament Heavy promoter, a mouse Neurofilament Heavy promoter, or another Neurofilament Heavy promoter that drives expression of a transcript in RGC of a subject. A Neurofilament Heavy promoter may be a long-form Neurofilament Heavy promoter or a short-form Neurofilament Heavy promoter. A subject in these examples may be a mammal, or a human, or a mouse, or a rat, or a dog, or a cat, or a horse, or a cow, or a sheep, or a pig. Examples of nucleotide sequences of the foregoing promoters, any of which is explicitly included as a possible example for all examples disclosed herein, are given in Table 1.

TABLE 1
examples of RGC promoters

| SEQ ID NO: | Promoter | Sequence |
|---|---|---|
| 1 | MOUSE | GGTCCCATGCCACTAGTGGGAGCTGTGTTACCTGTTGCA |
| | Gamma | GCCCCACCCAAAGCCCCTGCTATAGGTCAAGCAGGAATC |
| | synuclein | ACCCTGCCATCCCCAGCCTGGGGCCTGGAGTACCAGATC |
| | | CAGGAAACTAGCATCCCTTAGCTATAGAGATAGCCACAC |
| | | ATCAGCCCATTCCTCAGATGTGTATCTGGGGCTCAGACA |
| | | TCATCTCCCGATCTCCGACAAGGGCAGGATTTCCTTACC |
| | | GTCTGATGGGGTCTCTGCTGGTATCCTCAGCCCCTAGTC |
| | | TCCAGCCTTCAGGCATGCCGGGCCTATTGAGATGGGAGA |
| | | ACTTGGTACCGGGGTCCTGTGCCCAGGACCCTAGCAGTC |
| | | CCCAGCTCAGGTACACCCCAAAGCCCAGCAGCAGTGTCG |
| | | GGATCATGGTGAGGGGCTCCTGTGCTGATGCTCAGCCTT |
| | | ACAAGTGACTCTCAAATTTGCTGGTGATGTGGTCTTCAA |
| | | GCGAAATGTCAGAAAGAAAAGAAAACACGAGGACAACAA |
| | | AGGGAGGAAGTGGCCTGGTCCGGCCCACCCGGCAAGTCT |
| | | CATCCGCCCCCGCCCCCGCCCCTTCCAGCCTGGCCCCCT |
| | | TGGAGGCCTCCAACCACTCAGGTCAATTCCTGTGTCCTG |
| | | AGGGCACTTGAATCAGGGACACGGGATTTGGTAGACACA |
| | | TAAAGGTGGCCCCATTAAACTTATTTCTCCAGGACTCTG |
| | | TCGTGGGCCTGGAGGAGATCTGGTGCCACCCATACTGTT |
| | | GGCCAGGAAGTGGGGAACGGGCACATCACACCTGCTCGG |
| | | CACCTTGGGCTATGGGAACTAGCAGGTGGGTGGGAACTC |
| | | AGAGAAGGAAAGGGACTATGCTAGAATCACACAGCGGGC |
| | | AGCCCAGTCTAGGGCATGGGGAGCAGCTTTGGGTGTTTC |
| | | TGGCCTCAGCCTTCCAACAGGTTTGGCTAGAGCTCCAGG |
| | | CTCAAGAGCATCCAGGATACAGTGGGGAACTGGATAACA |
| | | GGGCAGCCTGCAGGTTGGCCATTCATTGGATTGGCCCTG |
| | | ACCCCGGCCCAGCCTGGGACACTGAGGCATCATCAGTCA |
| | | AGGCACTTTTCTTCTGCATATAAGAGCCAGGGCACGAGA |
| | | CCACCAGGGCTTTCCAAGGATGAATGAGGTGTAATGATA |
| | | GATTAGGATATGTCCAGCCTCCAACACGCTCTCCCTCCC |
| | | CCAGGGCCAACAAGAGTCAGCAGGGCAGAATAGAGCCAG |
| | | TAGGGGCCCGGGCCCTGCTCGCTGGTATCCCCGTGAGGC |
| | | ATGCCTTCTCTCTGGCCCGCCCTCCCTGCCCCCACCCTG |
| | | GCCCGGGCTGGCTGGGCTCCAGCCAGCAGCCACAGCATC |
| | | AATATTTCATCTGCGTCAATAAGAGGCAGTAGCAGCAGA |
| | | GACAGCGGCTGCGGCAGCACTCCAGTCCATAGCTTGCAG |
| | | CAGCCAGGTTCCATCCTTGCAAACACCATGGACGTCTTC |
| | | AAG |
| 2 | MOUSE | TGAGCCTTCTTATTCAGACCACCAAAATTACTTTATTTT |
| | Synapsin 1 | TCCACATGAAAGTATTATGTGGCTTCCTGTCTGCAAAGA |
| | | GGAAGACATCCATGAACACTAATGACACTGGGTTTGGGC |
| | | TATGTCCGGAGCAGAGGAATGAGGCCATGTAGACTAAAT |
| | | ATGTGCATGTGGAGGAGGCTGAAAACACATCAGAGCTAG |
| | | CGCTGCAGGAAATGCTTCTGCATTGCATACCCAGAGTTT |
| | | CCTTGCTCATCTGGGAGTCTGTGTTTTTCCTAGATGTGT |
| | | GCACTTGTGTGAGATTCTCTGGGTGTGAGTCAAAGTGTT |
| | | ATCTGAATGTGTAATGTGTGCTCAATATGCTCATGTGTG |
| | | TTACCCTGAGCTTCTGTGTCTACATATATACCTGGATGC |
| | | CTGTGTGTTCTGTGATGTACATATATATTCTGTCTTTCC |
| | | TTCCTTTTCTATTTGTGTTATTCCATGTGTTCTTTCAGA |
| | | TTCTCACCACCAAGGGCAAGGATATGTTAACTACCCAAG |
| | | TGTCCACCTCCGCCTGTCTGGTGATGTTTACGCCACCCC |
| | | CGTGCTCTTTTCTTTGCCCGACAGAGTTGTTATAGGAGA |
| | | TGTCTCCCCGGGAACACTGCAGGAAGGAGAATTTCTACA |
| | | TTTATGTTCCCCTCTGAGTGTGCTTCTATCCCCAAAATG |
| | | CCTTCAAAGGTGAAAATCAACACTGGAAACCCAAGTATC |
| | | TGGGAAGGGCAAGAGTGTGTAAGTGCAAGTTAGCCTAAG |
| | | GAATAGGAAGAGGTTGGTAAACAGGGTAGGATCGTGGGA |
| | | GGGAGTTTCGTTACTACAGGTCCGGACCCTCAGGACAAG |
| | | AACCCCACCCCCACTCCCCAAATTGCGCATCCCCCGCCC |
| | | CCATCAGAGGGGGAGGGGAAGAGGTTGCGGCGCGGCGCA |
| | | TGCGCACTGTCGGATTCAGCACCGCGGTCAGAGCCTTCG |
| | | CCTCCGCTGCCGGCGCGCACCACCACCTCCCCAGCACCA |
| | | AAGGCTGACTGACGTCACTCACTAGCCCTCCCCAAACTC |
| | | CCCTTCCTCGCCGCCTTGGTCGCGTCCATGCTGCCGTGA |
| | | GTCCAGTCGGACCGCACCACGAGAGGTGCAAGATAGGGA |
| | | GGTGCGGGCGCGACCATACGCTCTGCGG |
| 3 | HUMAN | ACTCAGTCCTTTTTGTGCTGTCTCCTCCTCTTTCCCAGA |
| | Thy-1 cell | GTTCCTCTCTCTCTTCTCCCACTAGGCAGGGATGAGCAA |
| | surface | GAGGAATGGCTCACCCTTGAGAGCTGGGGTCCATAGCCC |
| | antigen | AGGTCAGTTCTCCAGCTCTCCCACTTACCAGCCAAGACA |
| | | GGAGGTGAGGATTGAGATGGGATGAACCCAGCAGGCGGC |
| | | CATGGGTTAAAGGTCGCCATGAATGTAATGTGCCCAGCA |
| | | CAGTGCCTGCTAAAAGGCAACACTCCCTTCCTGGTCTGA |
| | | AGACCAAACAAGCAGACTGTACTCAGGAAAGCCAGAAGA |
| | | ACCTTCCAGCTGTCTGGACCAGAAGGTGCCAGCCCAGGG |
| | | GCTGAAGAAGACGTAATGCCCAGAGCAAAAAGCGCCTGC |
| | | AGCCCCCTGAAGGGCTGGGTGCTCTGGAATAGATGAGGG |
| | | GGCGAAATGGGGCTGGGGACCAGGGACGGACAGGGTGGG |
| | | TCCAGCACCTGCCTCGCTTCCGAAGGGCTGCTCCAACAC |
| | | TGAAAAACACCCAACCAGCTTCCTTTCAGAAAGACTGGA |
| | | ATATTCCAAAACTTCTCACTGGAGGCTCCGGAGGAGGTG |
| | | GGCTCCAGCTGAAAAGGAAATGTGGAGGCGTGGGCGCTC |
| | | CCGGCCTGCATCCTGCACCTCTTACACTTTGGTTTTCCC |
| | | ACAGACTCCTGAAGAATAGGTCAGAAGAAAGGGTTAAAG |
| | | CCTTAAAAGGGGAACAACCATTGCGGGGCTCAGGGAGGA |
| | | GGATAATGTTCTTTGGGCTGCCGCACCCTGATCCCCGGG |
| | | GTCCCGAACCCTCCCGTCCCTGGCCAGGCCTGCCAGCCA |
| | | CAGGGTGAGGGCCCCCTTCCGCCGCAACCTGCCACTCTC |
| | | ACACCAATGCGGGACCGCCTTCTCTTCCTTCCCCACCCC |
| | | CCACCCCACCCTGCCGTCCTTTCTCCCCCAATCTCCGCC |
| | | TCTGATTGGCTGAGCCCCCGGCTCCCCGCTCCCCCTCTC |
| | | CTCCATCCCCGGTGAAAACTGCGGGCTCCGAGCTGGGTG |
| | | CAGCAACCGGAGGCGGCGGCGCGTCTGGAGGAGGCTGCA |
| | | GCAGCGGAAGACCCCAGTCCAGGTGGGAACTGGAGCCGG |
| | | TGGGACCTGGGGCTCGGGGACCGCCGTCAGGCGCCCATG |
| | | CAAGACTTCCCAACACTAGGCTTCGGGCCACGGTCCGAG |
| | | GGCGCCCAGGGAAGAAGGGCGCAGAGCTTAGGGAGGGGC |
| | | CTGCTTTCCAGGCAGGGGGGGGAGGGGGATGCTTCTGCA |
| | | GGGCAGGGGCCGCGTGGCACCCTGATGTCTTTCGGGGAA |
| | | GGCGCTCCCGGGCTTTCGCCCGCTGGGGGACTGGTGTCT |
| | | GGGGCTGGGGCGCTGGAGAACAGGGAGGAAGGGCACCAA |
| | | GGACAGCCTGTGGGTCTACATTCCACCCAGACGTCCCCA |
| | | AACCCAGCTCGCAGAGGCGGGGAGGAGGACGGATGAAAC |
| | | TGCGGGGAGAGGATGGAGGATGGCGAGCTAGAGGGAATC |
| | | TGCCGGGTGACCTCGCGGCGGGCTGGGTGCGGGGCACCG |
| | | GAGGAGAAGGAAGCCGCAGTGCCGCAGGCGGGGACTGGG |
| | | TGGAAGGCGGGCGGACGGGGGAGGGGAGAGCTGGAAAAG |
| | | GATGAGAGAGGGGGAAGGGGGACTCATTTGGGAAAGGAG |
| | | AGGATTGGAATACGGAAATGGATTAAGGATGAGGCCCGC |
| | | CGGGGGCTTGAGAGGGAGGAAGAGCAGACCTTCTCTGGG |
| | | TCTGGAGCCGCCTGAGGACACAGACCAGAGGAAATGAAT |
| | | ACAGACTGCACCTCCCCAGCCGCTCTCCACCCCTCCCCT |
| | | GGCTCTTCTACCCTCTCCAGCCCCAGACCCATTTCTTCC |
| | | CTTTCTTGCTCTGGCCATTGCTCCCCCTTCCCCTCCTAG |
| | | ATCCCAAGCCCGCACAACATCTCAAACAAGAGTCCTCGA |
| | | TTCAAAAGCCAGATGCCGACCCCCCTTCCTCCTGGATCT |
| | | GGCTCAGGGCAGCAGCTCCACCCCGGGACAGAGAGAGCA |
| | | TTGATTGTAGCTGCAGCCGCCGCGGGATCCTAGCCTCAC |
| | | CCGTCAAGGGGCTGAGCGCCAGGGACCCTGAACTCGTCT |
| | | AGTGGTGCGCCCTGCGCACCCGGGCGCACTCAACCGAGG |
| | | CAATGCCCTGCGCGCTCTCGCGGGTGCACGCCCCTTCTG |
| | | TGGCCTCTCCTGGGCGAGCACTGCTCTGCAGATAGGCTA |
| | | GACTACCGGCTCCGCGTCGCCTCGCCAAGGGTTGGTTCA |
| | | GCCAAGGCTGCAAAAAACAAAAAAAGACCAGGCAGACAG |
| | | CCTATCCAGGGTGGCTATTGAAACTGGGCTGGAAAACTG |
| | | CAGTCCCAGGAACTCCAGAGAGCTGGACATTGGGAAGCA |
| | | TCCTTGGCTCACATACAATCGGAGATCACTATGTCTTTC |
| | | TCTCCTCCAGGAACACGATTAGCTTGTGTCCTATCCAGA |
| | | TAGGAATAGATGCTCCCTATCTGGGAGCATCCTTAGCTA |
| | | TGGTGAATGGTATCTAGCCATCCACTGGGGATGGCGAGT |
| | | GACTTAGGGATTTGTGTCTCACGTATATGAAGCAGTCAT |
| | | CGCCAGATGTTGGTTGTTTTTCTTAACCCCCATCATAAC |
| | | CCGGTGGGTATGTAAGATTCAGAGAGATTCATTCATTCA |
| | | TTCACAATAAATATCTTTGGAGTGTATGCTATATGCCAG |
| | | TAATCTGCAAACGGAAACGGTTTTGAGCATTGGGGATTT |
| | | TCTTCTGAACAGGAAATGGGAAGTCCCTAAATGGGGAGT |
| | | CTTTGTTTAACAGATACAGAGTTTTACTTTGAAAGACAA |
| | | AAAGAGTTCCGGAGATGGGCTGCATAGCAACGTGAATG |
| 4 | HUMAN | CATAGGCAGCTTCAATCTGATGGCTGTGGCCCCTTGGCC |
| | Neuro- | TCAACAGAATACATCTTGGAGCCCCCTTTTTACCCCAAA |
| | filament | CCCCCATTCCTCCTTGCTGTCAGCTGCTTGTGAGCCTTC |
| | Heavy | TCACATCCAGAGAATGTATCAGCATTGTGCAGACTGAAA |
| | (LONG | AGACCCAGAGGAACAAGGCTCCAATGGCAAAATTCCAAG |
| | FORM) | TAGAATGACAAATAAATGGGGAGCCATCTGAGAGCAAGG |
| | | GAGTCCTGCCCAACACCCGCCCCATGCCTTTCTCAGGGA |
| | | CCTCAGACCAGCCACTCACCTCCATCCTCCCAGCACCAC |
| | | CTGCAACCAGCCCCTTGCCCTCTGCAAACTGGAGCACGA |
| | | CTGGATCTTTAGATGGGGGAAAAATGCTTCATCATGTTC |
| | | TGCTGCTTCATGCAAAACCAGAAACTCCCTCCCCCTCTT |
| | | CCCTCCTCCCAGCGCACTCTCCTTCCAGTAAAAAGTGGT |
| | | TAAAGGGACAGCGCCATCAATTTCCCAGCTCTGAGGGTC |
| | | TGCTTAGAACTAGGGGGCTGGAAGGAGACAGAGGGCAAA |
| | | GAGAAAGGAACTGGCAGAGGTCTTTCCTGGGGGATATGT |
| | | CTGTTCTGTCCTGGGGATCCTGGAGCAGGAAAACCCGCG |
| | | TAAAGTAGGGGTGTAGTGGGTGTTGAGATAACTGCCTGG |
| | | GGGAGGTTCAGAGTGGAAGTACGAGTCTACAAACTCTCA |
| | | AGGGCGTCTCAGGGCTCCCAGCATCCCCAGGGGTCCTTT |
| | | CGCAGGGGTCCCTAAGCAGGAGGGGAACAGCCCAGAAAA |
| | | CACGGAACTGGACCCCCGACAGGAAGTCCAGGGAGGGGT |
| | | CCCTGGCTCACTATGTGACCCTGCTGGATCACTTGCCTC |
| | | CCCTCTCGGGTCCCCTCAGCACAGTGTCCCTCCCTTCCT |
| | | TCCCCTAAAGTAAAAGCAGAGGGTTAATCTCTTTCCCCG |
| | | CCCCACGCCCAACAAAGAGCAGGCCCTGTCCCCGGTGCT |
| | | GAAGCGCCAGCCGCAGCACCACCCCCACTCCCACAGCAT |
| | | AAAACATGAGCCAAAACCAATAAAGAGCCAAATGTCACA |
| | | GCCGTTGCAGGGCCCCCTAAATCCTGGGGACCCCTTCTT |
| | | CTACCTGACATCCTATTGGGGTGAGGGACTTTGGTACTC |
| | | AGAAAGCATCTCATCACTTCCCTGTAAGAGAGAAGGGAT |
| | | GCCGACTCAGGCGCCTGCTTGTCTGTTACAGGAGTGGGG |
| | | GAAGAGAGGACAAGTTGAGGCTGAGAAGATGGGGAGGGG |
| | | GAGGGAGAAAAGAGGACTTCCTAGTGTTGACAGAACGGC |
| | | AAGATGTGGGTTCCCCATCCCCAGTTCAGCCAGAGACCC |
| | | CTCAAAGTGGAACTTCCTGGGGCAGTCGGGGGTCAGGAG |
| | | TTGGAGCTTGTCTCTGGGGCAAGACCCCTTCGTTGTACA |
| | | GATGGAAAAACAAGGGTGGGAGGACACAGCTTGTCCAAG |
| | | GTCATTCGACCAGCAAACTGCCTAGCTGACCCCAGTGTG |
| | | CAGAAGCTGGCTCGGGTGACACCCATCATTTCCCCCCAC |
| | | CCCACACAGGGGCCAGCTCTCTCAACTTCATGCCCAAGC |
| | | CCTCCTACGGTACCCCCACTGTAGGTTCTCTGCCCCTCA |
| | | AACTCAGCCCAGCTTTCTCCTGCCTGTTCAGGGGACCTT |
| | | CTGCCCGCTTCGCTGAGGGTCCGTCCCCTTTACTGGGGC |
| | | TGGCAGCAGGGTCTCCCATCTCCTCTCTCGGGGGCCACT |
| | | GCAGACTTTTTAGAGAACGCCTTGCCTCCCCCCAACCCC |
| | | ACCCATCCGGGGTTCCCTCTCTCCATCCTCTGCAGTGTC |
| | | TCCCATACCCCCATTCAGGGTAGCCTTGCTATTCTCCCC |
| | | AACTCCAGGTCCCCCTTCATCTATTCCGGGGCTGGCCGC |
| | | GGAGTTTCCTGAGCGCTCTCCAAGTGGGTCCTCTAGATG |
| | | TTAGGAGAACACTGTACCTCCCCCGGTCAGGGGTCTCCT |
| | | GTCTCCGTTCTATGGAGCGTCCATGCTCCCATTCAGGAC |
| | | TGCCTTGCTCCCTCCTCTGTTCCGGGGCTGGCTGCACAG |
| | | TCTCTGCACCCCCTATCCTGAAAGCCTCTCTTAACTATT |
| | | TGGAAAGCCTCGTGTCCTGTCTCATACAGGGATCCCCTC |
| | | ATCCTAATGACTGCAATCTTCCATTGCTCCATCCCGAGG |
| | | GCATCCTGCCCCTATTCCCATCAGGTTTCTCCTTGTCCT |
| | | CTCCCTGTTTCAAGTCCCCTTTCTTATTCCGAACACACT |
| | | CGCAGGCTCTTCCGACGCGCACCCGGGGGTCCTCACTGG |
| | | CCCACTCCGGGAGTCCTCTGCCCGCTTCCCCGACCTCGA |
| | | GGGTCTCCTCTGACGCAGCGTCGATTCCCCTTCCCTCCT |
| | | CGGTCCCCTGCCCCGCCCCTCTCACTGCGGCGGAGCCGG |
| | | TCGGCCGGGGGGCCGCAGGGGAGGAGGCGGAGAGGGCGG |
| | | GGCCCTCCTCCCCACCCTCTCACTGCCAAGGGGTTGGAC |
| | | CCGGCCGCGGCGGCTATAAAAGGGCCGGCGCCCTGGTGC |
| | | TGCC |
| 5 | MOUSE | GCCTTGGCTGTCCTGGAACTCACTCAGGCTGAGTGAGGC |
| | Neuro- | TGACTTCAGATTCACAGAGATCTGCCTGCCTGCCTCTGC |
| | filament | CTCCTGAATGCTGGGATTAAAGGCGTGCGCTACCACTGC |
| | Heavy | ACAGCAAAAAGAATCATTCTCAGCTCTCTCTGGGCCATA |
| | (LONG | CGTTTTGCTAGAGAGCTGATTAGAATTCATCCATCTATC |
| | FORM) | CACTCACAATGACAAACTGGGAAGCAGCATGCGGGCAAG |
| | | GACCACAGAACCCCAGGAGGGGACAAGGCTCAGGGTGAA |
| | | GGGAAGGTGAGGCTGAAGGACTGACCAGGGTCTCAGCAT |
| | | TCATGACAACCTTACAGCCACAAAAGCCACACCTTTTAC |
| | | CTTCACACACACCCCACTCCTATCATTCATGTGTGCTGT |
| | | CAACTGCTTGTCAGACTTCTCACCCCCAAGAAGGGCATG |
| | | TGCATTCTGCAGACAACTGAAGAGACTCGAAGGAACAAG |
| | | AATCTAATAACAAAAATCCAAGCAGTATGGGAGATAAAT |
| | | GGGGAAGCCATGTGGGCGTAAGGGGGTAGAGGTCTGCAT |
| | | CCCAGTCCCCTCCCCATGGCATCTGCAGTGCCTCCCAGC |
| | | CTTTCTGACCCCTGCAAAGAGCAGCATGACTGGACCTTT |
| | | AAATTGGGAAAATGCTTCATCATGTTCTGCTCCATCATG |
| | | AAAAACTAGAGTCTCCTCCCCCTCCTCCCTAGTGCACTC |
| | | TCCTGGCCTGCAGCCAGGGGCTGGGAATGAGACACAGGA |
| | | CAGGAAAGGGATCTCTTTTAGGGAATCTATCAGTTCTCC |
| | | TCCTAGGGATCCCTCCAAAAGAGAAAACCACAGCAAACT |
| | | GGGGTGCAGTGAGGCTTGAGGTAACTGCCTGGGAGAAGT |
| | | TCTGATCTGAAGAAGTCTATACTGGTTTCCAGAGCTTGT |
| | | CAGTGGGCATTGGAGTGGGGCTCTCTCTGCTCCGGGAAG |
| | | AGGTTTGCAGGGAGAAAGAACTTCACAGAGAGCCAGGCA |
| | | CTGGACAGGACATGCAGGGGTGGGTCACTTACATACAAC |
| | | CGTAGGTCGTTTCGAGCCCGTCATATGACTCATCCAATC |
| | | CTCCCCTGTACCGCACAGAGGGACTGCTTGGAAAAGCTA |
| | | TGGAACCTCCCTACTCCGTTAGGCATAGATTTAACCCTT |
| | | CCCATCCGAGGAGCGGCTGCTGTCCGTGGTGCTGAAGCG |
| | | ATAGCGGCACGGGCGGCTCCGTCCACTAACACCGCTTTT |
| | | GACCGGAAAACCAAACCAAGAACGAGCCGTATAATAAAG |
| | | CAAGAGCTCCAAGTCTAAGCCCCTCCGCCGTCCCCGCCC |
| | | TTTCACCTGAAGCCTCAGTAGGGCTCATGATGGAGGTCG |
| | | GTGGACTTTGGTACTGAAAAACCACTCCACCACTTCCTC |
| | | GGAGCATGAAAGGGGATGCTTACGGCAGTACTGGTTCAT |
| | | CTATTCTGGAAAAGGAATGAGATGCCAAGATAAAGCAGA |
| | | AAAATCGGGCAAGGAAGGGAGAAAGACAAAGTTCTCAGG |
| | | TGAGAGGAACTGGTTACTATTCCGACTGGCAATATGTGG |
| | | GTTCTCCTCCCCAAAATCAGCCAGACATTTCCCAAGTTC |
| | | GAACCTCCTAGGGGCACATGGGAGCTTGGAGCTGCATCT |
| | | TGTCTCTTGCACACAAGGGAAAACCAAACATAGGAGAAC |
| | | ACAATTTGTACAAGGTCATTCAGCTAGCGAAGCACAGAA |
| | | GCTAACCCCACCCTGTGGCAGAACTTGGCTTCGGTGTTG |
| | | AGGCTCTTGCTGCCTACTGAGGGACCCCCTGTTCTTCGT |
| | | AGGCAGTTTTCCTTTCCGGGCAAGAGGAGACTCCACTTT |
| | | CCAGTCGTGGCCACTGGAATTTTTAGAGAGCACCACGTT |
| | | CCTCTCACCCAGCGCTCCCTTTCTCCGTCTGCAGTGTTC |
| | | TCCTTCTCAGGGTAGCTTTGCGGTCCTTTCAAACTCCAC |
| | | GCCCACCCCAACCCCAACCCCGAAGCCAGCTGTACAGTT |
| | | CCTTAAGCCCCTTTGGGTGGCCCAGGGCCGCTGTAGTAT |
| | | CTGGGGAACACTGCACCGCCAGCTAGAAGGTCCCCATTT |
| | | ATCATCAGTAGCATCCATCATGCAACCCCATACAGAATC |
| | | CCTTCGTGGGTGACTGCAGTCTGCACTCCTCATCTCAAG |
| | | GTCCTCTCTAACTATCAGGGAACCAACCCTGTGCTGCTT |
| | | CTCAAGTGGGGGTGTCCTCTCATAGTAATCACTGCAGTC |
| | | TCCCACTGCTTCAACCCGAAGGCGCCCTGACCCATCAGT |
| | | TCTGCAATCCTCTCCCTATTTCCAGTGCCCTCTCTTATT |
| | | CTGAGGGTCTTATTCTGACTAATAGGGTCTTCCGACATG |
| | | CACCTGGAGGTCTGCACTTGTCCGCTCCGGAAGTCCTTT |
| | | ACTCCTTGGTCTGACCTCGGGAGGCTCTACTGACGATGC |
| | | GTCGATTCCCCTTCACTCCTGGGTCGTCCCCCCCAGCCC |
| | | CGCCCCTCTCACTGCGGAGAAGCCGGTCGGCCCGGGGCC |
| | | GCGGGGGAGGAGGTGGAGAGGGTGGGGCCCTCCTCCCCA |
| | | GCCC |
| 6 | MOUSE | CCCTGCCCCGCCCCTCTCACTGCGGCGGAGCCGGTCGGC |
| | Neuro- | CGGGGGGCCGCAGGGGAGGAGGCGGAGAGGGCGGGGCCC |
| | filament | TCCTCCCCACCCTCTCACTGCCAAGGGGTTGGACCCGGC |
| | Heavy | CGCGGCGGCTATAAAAGGGCCGGCGCCCTGGTGCTGCCG |
| | (SHORT | CAGTGCCTCCCGCCCCGTCCCGGCCTCGCGCACCTGCTC |
| | FORM) | AGGC |
| 7 | HUMAN | CCAAGGTCCAGGCCCTCTTACCGTCCTACGGGGTCCTTG |
| | Gamma | CCGGTGTCCTCAGCCTCTGCCTTCCAGACCCCAGGTGTC |
| | synuclein | CTGGAGCTCTGCAGATCAGAGAGGCTAGTACTGGAAGCC |
| | | TGGGCCCATGCCCCCAGCAGCCCCCAGCCCAGGGGGCCC |
| | | CCAAGGCTGAACAGCAAGCTCAGGATCATCTTGGTGGTG |
| | | GGGCAGGCTCAGCTCACACTCAGCCTTGGCAAGTAGCTC |
| | | CAGAAACTGCTAGTGACGTTGTCTTCAAGTTAAATCTCA |
| | | GGAGGAAAAGAAAATACGAGGACAACAAAGAGAGGAAGT |
| | | GGCCTGGGCCGGCCTACCCGGTGGGTCTTGTCCTGCCCC |
| | | CCAACTACCCTGGCTGGCCCCACAGGGGCCGCCAACCAC |
| | | ACAAGCCAGTTCCTGTCCCTGAGGACTTGGCTCAGGGAC |
| | | TCTGGGAATGTGGTAGACATGGGGTGGCCCCACCAAATG |
| | | CATCCTTATGGGAACCTGCTCCCTGGGAGCCATGAAAAG |
| | | AGCGTGGACTTCGAGGTGGGGCCACAGGAAGTGGTCAGG |
| | | TCCATCTCAGGGGACCTGCTGCCCATCCACACTGCTGGC |
| | | CAGGAAATGGGGGGCAATTCATGCCTCCTCAGCACCTTC |
| | | AGCACTGGGCGGCTCAAAGAAGGGAAGGGACTATTCTGG |
| | | GGTCACACAGCATGCAGCCAGAGGCCAAGGCATGAGGAA |
| | | GTCCTTCATTTCCCCACCCCCACCCACCTCAGATCCTCC |
| | | AACCGGTTTCATGGCAGCCCAGGGTCCAGCGGCATCCAG |
| | | GATGCTGGTGGGTAGCTGCACAGCCCAGGCCGCGGGAGG |
| | | TTGGCTGCTCTCACCTAACAGGCCTATGTGGCCCTGACC |
| | | CCTACCTAGGAAGCTGGGGACAATGGCCAAGGCGCCTCC |
| | | CCTCTCTGTGCCTGTCTGTCCAGGTGCAGCATAGACACA |
| | | GCACCCCTGGGGCCAAGAGCACCCAGCCAGGGCTGCCCC |
| | | CATGGGTGGGCAGGGCAGTAAATGAATGAGGGACAGGTT |
| | | GGGAGGTGGCCAGCCCCCTCCAGCCCATGGAGGGCACGG |
| | | GGCAGGAGAGCTGGGCTGAGCCAGCAGGAGCCCAGGGAG |
| | | CCTGGTCTCTGCCTTCCTATCCTGGAGGAAGGTGAGGCT |
| | | GAACCTCCTTCCCTCCCTCCCTCCCTCCCCGCCCCCACT |
| | | GCACGCAGGGCTGGCTGGGCTCCAGCTGGCCTCCGCATC |
| | | AATATTTCATCGGCGTCAATAGGAGGCATCGGGGACAGC |
| | | CGCTGCGGCAGCACTCGAGCCAGCTCAAGCCCGCAGCTC |
| | | GCAGGGAGATCCAGCTCCGTCCTGCCTGCAGCAGCACAA |
| | | CCCTGCACACCCACCATGGATGT |
| 8 | HUMAN | ATGAGTGCAAGTGGGTTTTAGGACCAGGATGAGGCGGGG |
| | Synapsin 1 | TGGGGGTGCCTACCTGACGACCGACCCCGACCCACTGGA |
| | | CAAGCACCCAACCCCCATTCCCCAAATTGCGCATCCCCT |
| | | ATCAGAGAGGGGGAGGGGAAACAGGATGCGGCGAGGCGC |
| | | GTGCGCACTGCCAGCTTCAGCACCGCGGACAGTGCCTTC |
| | | GCCCCCGCCTGGCGGCGCGCGCCACCGCCGCCTCAGCAC |
| | | TGAAGGCGCGCTGACGTCACTCGCCGGTCCCCCGCAAAC |
| | | TCCCCTTCCCGGCCACCTTGGTCGCGTCCGCGCCGCCGC |
| | | CGGCCCAGCCGGACCGCACCACGCGAGGCGCGAGATAGG |
| | | GGGGCACGGGCGCGACCATCTGCGCTGCGGCGCCGGCGA |
| | | CTCAGCGCTGCCTCAGTCTGCGGTGGGCAGCGGAGGAGT |
| | | CGTGTCGTGCCTGAGAGCGCAGTC |
| 9 | MOUSE | GAGCAGATCTCCAGCCAAGAGGCAAAGGAATGGGGGAAG |
| | Thy-1 cell | CTGGAGGGCCTCCCTCTGGTTATCCAGGCTTCTGAAGGT |
| | surface | TCAAGCAAAGAAAGGGTTACAACCTTAAAAGGAGAGCGT |
| | antigen | CCCGGGGTATGGGTAGAAGACTGCTCCACCCCGACCCCC |
| | | AGGGTCCCTAACCGTCTTTTCCCTGGGCGAGTCAGCCCA |
| | | ATCACAGGACTGAGAGTGCCTCTTTAGTAGCAGCAAGCC |
| | | ACTTCGGACACCCAAATGGAACACCTCCAGTCAGCCCTC |
| | | GCCGACCACCCCACCCCCTCCATCCTTTTCCCTCAGCCT |
| | | CCGATTGGCTGAATCTAGAGTCCCTCCCTGCTCCCCCCT |
| | | CTCTCCCCACCCCTGGTGAAAACTGCGGGCTTCAGCGCT |
| | | GGGTGCAGCAACTGGAGGCGTTGGCGCACCAGGAGGAGG |
| | | CTGCAGCTAGGGGAGTCCAGGTGAGAGCAGGCCGACGGG |
| | | AGGGACCCGCACATGCAAGGACCGCCGCAGGGCGAGGAT |
| | | GCAAGCCTTCCCCAGCTACAGTTTTGGGAAAGGATACCA |
| | | GGGCGCTCCTATATGGGGGCGCGGGAACTGGGGAAAGAA |
| | | GGTGCTCCCAGGTCGAGGTGGGAGAGGAAGGCAGTGCGG |
| | | GGTCACGGGCTTTCTCCCTGCTAACGGACGCTTTCGAAG |
| | | AGTGGGTGCCGGAGGAGAACCATGAGGAAGGACATCAAG |
| | | GACAGCCTTTGGTCCCCAAGCTCAAATCGCTTTAGTGGT |
| | | GCGAATAGAGGGAGGAGGTGGGTGGCAAACTGGAGGGAG |
| | | TCCCCAGCGGGTGACCTCGTGGCTGGCTGGGTGCGGGGC |
| | | ACCGCAGGTAAGAAAACCGCAATGTTGCGGGAGGGGACT |
| | | GGGTGGCAGGCGCGGGGGAGGGGAAAGCTAGAAAGGATG |
| | | CGAGGGAGCGGAGGGGGGAGGGAGCGGGAGAATCTCAAC |
| | | TGGTAGAGGAAGATTAAAATGAGGAAATAGCATCAGGGT |
| | | GGGGTTAGCCAAGCCGGGCCTCAGGGAAAGGGCGCAAAG |
| | | TTTGTCTGGGTGTGGGCTTAGGTGGGCTGGGTATGAGAT |
| | | TCGGGGCGCCGAAAACACTGCTGCGCCTCTGCCAAATCA |
| | | CGCTACCCCTGTATCTAGTTCTGCCAGGCTTCTCCAGCC |
| | | CCAGCCCCAATTCTTTTCTCTAGTGTTCCCCCTTCCCTC |
| | | CCCTGAATCTCAAGCCCACACTCCCTCCTCCATAACCCA |
| | | CTGTTATCAAATCCAAGTCATTTGCCACCCAACAACCAT |
| | | CAGGAGGCGGAAGCAGACGGGAGGAGTTTGAGATCAACT |
| | | TGGGCTACATCACGAGTTCCAGGCTCACCAAGGCTTCTT |
| | | AAGGAGACCTTGTCTCTAAAATTAATTAATTAATTAATT |
| | | AATAGTCCCCTTTCTCTGCCACAGAACCTTGGGATCTGG |
| | | CTCCTGGTCGCAGCTCCCCCCACCCCAGGCTGACATTCA |
| | | CTGCCATAGCCCATCCGGAAATCCTAGTCTATTTCCCCA |
| | | TGGATCTTGAACTGCAGAGAGAATGGCAGAGTGGCCCGC |
| | | CCTGTGCAAAGGATGTTCCTAGCCTAGGTGGAGCTCGCG |
| | | AACTCGCAGACTGTGCCTCTCTTGGGCAAGGACAGGCTA |
| | | GACAGCCTGCCGGTGTGTTGAGCTAGGGCACTGTGGGGA |
| | | AGGCAGAGAACCTGTGCAGGGCAGCAATGAACACAGGAC |
| | | CAGAAAACTGCAGCCCTAGGAACACTCAAGAGCTGGCCA |
| | | TTTGCAAGCATCTCTGGCCTCCGTGCTTCTCACTCATGT |
| | | CCCATGTCTTATACAGGCCTCTGTGGCACCTCGCTTGCC |
| | | TGATCTCATCCCTAGCCGTTAAGCTTTCTGCATGACTTA |
| | | TCACTTGGGGCATAATGCTGGATACCTACCATTTTCTTA |
| | | GACCCCATCAAAATCCTATTTGAGTGTACGGTTCGGAGA |
| | | ACCTCATTTATCCGGTAAATGTCTTTTACTCTGCTCTCA |
| | | GGGAGCTGAGGCAGGACATCCTGAGATACATTGGGAGAG |
| | | GAGATACAGTTTCAATAAAATAATAGGTTGGGTGGAGGT |
| | | ACATGCCTATAATGCCACCACTCAGGAAATGGTGGCAGC |
| | | TTCGTGAGTTTGAGGCCAACCCAAGAAACATAGTGAAAC |
| | | CCTGTCAGTAAATAAGTAAGCAAGTATTTGAGTATCTAC |
| | | TATATGCTAGGGCTGACCTGGACATTAGGGGTCATCTTC |
| | | TGAACAAACTAGTGCTTGAGGGAGGTATTTGGGGTTTTT |
| | | GTTTGTTTAATGGATCTGAATGAGTTCCAGAGACTGGCT |
| | | ACACAGCGATATGACTGAGCTTAACACCCCTAAAGCATA |
| | | CAGTCAGACCAATTAGACAATAAAAGGTATGTATAGCTT |
| | | ACCAAATAAAAAAATTGTATTTTCAAGAGAGTGTCTGTC |
| | | TGTGTAGCCCTGGCTGTTCTTGAACTCACTCTGTAGACC |
| | | AGGCTGGCCTGGAAATCCATCTGCCTGCCTCTGCCTCTC |
| | | TGCCTCTCTGCCTCTCTGCCTCTCTCTCTGCCTCTCTCT |
| | | GCCTCTCTCTGCCCCTCTCTGCCCCTCTCTGCCCCTCTC |
| | | TGCCCCTCTCTGCCGCCCTCTGCCTTCTGCCCTCTGCCC |
| | | TCTGGCCTCTGGCCTCTGCCCTCTGCCCTCTGGCCTCTG |
| | | GCCTCTGCCTCTGCCTCTTGAGTGCTGGAATCAAAGGTG |
| | | TGAGCTCTGTAGGTCTTAAGTTCCAGAAGAAAGTAATGA |
| | | AGTCACCCAGCAGGGAGGTGCTCAGGGACAGCACAGACA |
| | | CACACCCAGGACATAGGCTCCCACTTCCTTGGCTTTCTC |
| | | TGAGTGGCAAAGGACCTTAGGCAGTGTCACTCCCTAAGA |
| | | GAAGGGGATAAAGAGAGGGGCTGAGGTATTCATCATGTG |
| | | CTCCGTGGATCTCAAGCCCTCAAGGTAAATGGGGACCCA |
| | | CCTGTCCTACCAGCTGGCTGACCTGTAGCTTTCCCCACC |
| | | ACAGAATCCAAGTCGGAACTCTTGGCACC |

In an example, an RGC promoter may have less than 100% sequence homology with a promoter disclosed in Table 1. In an example, an RGC promoter may have at least about 60% sequence homology with a promoter disclosed in Table 1, at least about 65% sequence homology with a promoter disclosed in Table 1, at least about 70% sequence homology with a promoter disclosed in Table 1, at least about 75% sequence homology with a promoter disclosed in Table 1, at least about 80% sequence homology with a promoter disclosed in Table 1, at least about 85% sequence homology with a promoter disclosed in Table 1, at least about 90% sequence homology with a promoter disclosed in Table 1, at least about 95% sequence homology with a promoter disclosed in Table 1, at least about 97% sequence homology with a promoter disclosed in Table 1, or at least about 99% sequence homology with a promoter disclosed in Table 1.

As disclosed herein, a vector may include a polynucleotide including a cis-regulatory element and a sequence encoding any CaMK, including without limitation any CaMK disclosed herein, including any constitutively active CaMK disclosed herein. As disclosed herein, a vector may include a polynucleotide including a cis-regulatory element and a sequence encoding any CREB, including without limitation any CREB disclosed herein, including any constitutively active CREB disclosed herein. In an example, any such cis-regulatory element may be a ubiquitous cis-regulatory element, including one or more of one or more enhancer and one or more promoter. A cis-regulatory element may be ubiquitously activity or promiscuous, meaning in can drive expression of a transcript in multiple different cell types, including as an example RGC. In another example, any such cis-regulatory element may be drive expression of a transcript in RGB but not in any other cell type or not in another cell type, or in RGC but not in other cells of the retina, or in RGC but not in cells of other tissues of the eye. Such a cis-regulatory element may be an RGC promoter.

Any combination of any of the foregoing cis-regulatory elements and a polynucleotide encoding any of the CaMK or CREB disclosed herein is explicitly included in the present disclosure, as is use thereof, including included in a vector, in any and all methods disclosed herein, without restriction. In an example, a polynucleotide including a cis-regulatory element and a sequence encoding a CaMK or CREB as disclosed herein may be recombinant. As used herein, recombinant means the cis-regulatory element of the polynucleotide and the sequence of the polynucleotide encoding the CREB or CaMK were created by splicing together sequences that do not occur naturally. For example, any such example may include a polynucleotide including a cis-regulatory element and a sequence encoding a CaMK or a CREB, wherein the cis-regulatory element is or includes one or more nucleotide sequence that is from a naturally occurring gene sequence other than the CaMK or CREB, respectfully. Ant vector as disclosed herein may include any of the foregoing examples, and any such example, including such vector, may be used in any of the methods as disclosed herein, without limitation.

In an example, activity of CaMK or CREB may be increased by contacting a cell with a vector, which vector may introduce the CaMK or CREB into the cell, or which vector may introduce a polynucleotide encoding a CaMK or CREB into the cell, leading to expression of the encoded CaMK or CREB. A vector refers to a macromolecule or complex of molecules comprising a protein, polypeptide, gene, or polynucleotide to be delivered into a cell. A vector may include, for example, a viral vector such as a retrovirus vector, a lentivirus vector, an adenovirus vector, an adeno-associated virus (AAV) vector, an alphavirus vector, a poxvirus vector, a herpes simplex virus vector. A vector may include liposome and other lipid-containing complexes, and other macromolecular complexes capable of mediating delivery of a polypeptide or polynucleotide to a target cell. Some embodiments of the present disclosure may include a vector comprising a recombinant nucleic acid.

In an example, a polynucleotide encoding a CaMK or CREB may by encapsidated within a recombinant adeno-associated virus. In an example the recombinant adeno-associated virus is of a serotype selected from one or more of AAV1, AAV2, AAV3, AAV4, AAV5, AAV6, AAV7, AAV8, AAV9, AAV10, AAV11, and AAV12. Other examples include viral vectors that are hybrids including components from two or more of the foregoing AAV serotypes.

Increasing activity in an RGC may include introducing a foregoing CaMK or CREB to an RGC such as by injection to a subject of a vector. Injection may be intraocular or systemic. Injection may be intramuscular, intracerebroventricular, intraperitoneal, subcutaneous, or by any other route that may result in contacting RGCs with the vector so as to increase activity of CaMK or CREB in RGC.

In an example, a method, incorporating any feature or features disclosed herein, alone or in any combination, may include decreasing degeneration of RGC. Decreasing degeneration of RGC may include decreasing one or more of a loss of number of RGC, death of RGC, loss of number of RGC somata, loss of number of RGC axons, and loss of RGC axonal projections including without limitation into the brain. RGC degeneration may include a loss of vision, including increased blindness. In an example, decreasing degeneration of RGC may include preventing or treating loss of vision. In an example, increasing CaMK or CREB activity in RGV prevents decrease in vision or visual ability, such as may be caused by excitotoxic or other insult or injury that may cause RGC disfunction or degeneration.

Degeneration of RGCs is a leading cause of visual impairment and blindness in a variety of pathological states. Some conditions injure RGC somata, including excitotoxicity and retinal ischemia, whereas others injure the RGC axon, including optic nerve transection, compression, papilledema and glaucoma or other instances of pathological or detrimental increase in intraocular pressure. Preventing RGC degeneration may prevent, diminish, or reduce reduced visual ability or acuity, or blindness, glaucoma patients including without limitation glaucoma patients who would otherwise progress to such impairments blindness even if given other treatment such as treatment to reduce intraocular pressure. In another example, CaMK or CREB activity may be increased in RGC of a subject wherein the subject possesses a genetic predisposition for the development or worsening of glaucoma. In some examples, a method may include increasing CaMK or CREB activity in RGC before RGC degeneration has occurred, or before it is believed to have occurred, or to prevent further RGC degeneration some degree of which may believe to have already occurred, or whether any degree of RGC degeneration is known or believed to have occurred but where the subject is believed to be susceptible to the development of such degeneration.

In some examples, a method may include increasing CaMK or CREB activity in RGC in a subject who has not experienced or been diagnosed with an impairment or decrease in visual ability or visual acuity or with blindness (which may include partial blindness or complete blindness), or in whom some impairment or decrease in visual acuity or ability or in whom some degree of blindness has been detected, diagnosed, or experienced by the subject, wherein the subject is believed to be at risk for development or worsening of such impairment, decrease, or loss of visual ability or acuity or of blindness (which may include partial blindness or complete blindness), such as to prevent, decrease, or lessen the development or worsening of any of the aforementioned visual impairments. In an example, such subject may have been diagnosed with glaucoma, or as susceptible to development of glaucoma, or an ischemic event or other trauma known, believed, expected, predicted, or having the potential to cause retinal degeneration or reduction in visual ability or acuity or development blindness (which may include partial blindness or complete blindness). Any one or more of any of the examples for increasing CaMK or CREB activity in RGC disclosed herein may be included in any foregoing method for decreasing or preventing RGC degeneration or foregoing method for treating vision loss in a subject.

A pharmaceutical composition as disclosed herein may include a polynucleotide and a vector, wherein the polynucleotide includes a retinal ganglion cell promoter and encodes a CaMK or a CREB. A CaMK or CREB encoded by such polynucleotide may include any of the aforementioned CaMK or CREB, without limitation, including a CaMKI, a CaMKII, a CaMKIV, a constitutively active CaMK, a CaMKIIα, a CaMKIIβ, a CaMKIIγ, a CaMKIIδ, a T268D substituted CaMKIIα, a T287D substituted CaMKIIβ, a truncated, N-terminal catalytic domain of CaMKIIα, a truncated, N-terminal catalytic domain of CaMKIIβ, a CREB, a constitutively active CREB, or a VP16-CREB. A polynucleotide included in such pharmaceutical composition may include a cis-regulatory element, such as a promiscuous cis-regulatory element, or a cell-specific cis-regulatory element, an RGC promoter, a gamma-Synuclein promoter, or other cis-regulatory element.

Some examples include a polynucleotide as disclosed herein, including a sequence encoding any of the foregoing CaMK or CREB, with or without any of the foregoing cis-regulatory elements, including without limitation an RGC promoter, wherein such polynucleotide is not part of a pharmaceutical composition. In an example, a vector, including any of the foregoing vectors, without limitation, may include such a polynucleotide, wherein such vector is not part of a pharmaceutical composition.

In another example, a composition, compound, agent, pharmaceutical, or other substance capable of increasing activity of a CaMK may be used to decrease degeneration of RGCs in accordance with the present disclosure. As an example, oleic acid (CAS 112-80-1) is known to stimulate CaMK activity. Given the present disclosure that increasing CaMK activity inhibits RGC degeneration and vision loss, as a CaMK activator, oleic acid would be expected to prevent RGC degeneration, and be used as a treatment for vision loss, as do other methods of increasing CaMK as disclosed herein. Explicitly included in the present disclosure are methods of decreasing degeneration of retinal ganglion cells in a subject, or of treating vision loss in a subject, including administering to the subject oleic acid, or a pharmaceutically acceptable salt thereof, or another stimulator or activator of CaMK activity or expression, or in place of a CaMK polynucleotide or vector in all examples of methods and in any subjects as disclosed herein, without limitation.

A pharmaceutical composition may include a formulation for administration to a subject. Such formulation may include any of those suitable for oral, parenteral (including subcutaneous, intradermal, intramuscular, intraocular, intravenous and intraarticular), rectal and topical (including dermal, buccal, sublingual and intraocular) administration. The most suitable route may depend upon the condition and disorder of a recipient or intended purpose of the administration. A formulation may conveniently be presented in unit dosage form and may be prepared by any of the methods well known in the art of pharmacy. Methods may include a step of bringing into association a CaMK or CREB or polynucleotide encoding CaMK or CREB, or vector including any of the foregoing, including any of the examples herein disclosed (“active ingredient”) with a carrier which constitutes one or more accessory ingredients. In general, formulations may be prepared by uniformly and intimately bringing into association an active ingredient with liquid carriers or finely divided solid carriers or both and then, if necessary, shaping the product into the desired formulation.

Formulations of the present disclosure suitable for administration to a subject may be presented as discrete units such as capsules, cachets or tablets each containing a predetermined amount of an active ingredient; as a powder or granules; as a solution or a suspension in an aqueous liquid or a non-aqueous liquid; or as an oil-in-water liquid emulsion or a water-in-oil liquid emulsion. Composition including an active ingredient may also be presented as a bolus, electuary or paste. For oral or other administration, an active ingredient may be suspended in a solution, or dissolved in a solvent, such as alcohol, DMSO, water, saline, or other solvent, which may be further diluted or dissolved in another solution or solvent, and may or may contain a carrier or other excipient in some examples.

In certain embodiments, an active ingredient may be incorporated with one or more excipients and used in the form of ingestible tablets, buccal tablets, troches, capsules, elixirs, suspensions, syrups, wafers, and the like. Tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

A tablet may be made by compression or molding, optionally with one or more accessory ingredients. Compressed tablets may be prepared by compressing in a suitable machine an active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. Tablets may optionally be coated or scored and may be formulated so as to provide sustained, delayed or controlled release of an active ingredient therein.

Formulations for parenteral or other administration include aqueous and non-aqueous sterile injection solutions which may contain anti-oxidants, buffers, bacteriostats and solutes which render a formulation isotonic with the blood of the intended recipient. Formulations for parenteral or other administration also may include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of a compound of Formula I to polymer and the nature of the particular polymer employed, the rate of a compound of Formula I release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

A compound of Formula I formulation may include different types of carriers depending on whether it is to be administered in solid, liquid or aerosol form, and whether it needs to be sterile for such routes of administration as injection. The present disclosure can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990.

As used herein, the term “effective amount” means an amount of active ingredient or pharmaceutical agent that may elicit a biological or medical response of a cell, tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of active ingredient, as well as salts, solvates, and physiological functional derivatives thereof, may be administered as the raw chemical. Additionally, the active ingredient may be presented as a pharmaceutical composition.

A pharmaceutical composition of the present disclosure may include an effective amount of a compound of Formula I and optionally one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains an active ingredient and optionally may include one or more additional active ingredient will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

Further in accordance with the present disclosure, the composition of the present disclosure suitable for administration may be provided in a pharmaceutically acceptable carrier with or without an inert diluent. The carrier may be assimilable and may include liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present disclosure is appropriate. Examples of carriers or diluents may include fats, oils, water, saline solutions, lipids, liposomes, resins, binders, fillers and the like, or combinations thereof. The composition may also include various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, a compound of Formula I may be combined with a carrier in any convenient and practical manner, i.e., by solution, suspension, emulsification, admixture, encapsulation, absorption and the like. Such procedures are routine for those skilled in the art.

In an example, the present disclosure may include the use of pharmaceutical lipid vehicle or a vector compositions that include an active ingredient and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples may include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, an active ingredient may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. The dispersion may or may not result in the formation of liposomes.

The actual dosage amount of a composition of the present disclosure administered to a subject (e.g., an animal or human patient) can be determined by physical and physiological factors such as body weight, severity of condition, the type of disease being treated, previous or concurrent therapeutic interventions, idiopathy of the patient and on the route of administration, and purpose of treatment. Depending upon the dosage and the route of administration, the number of administrations of a preferred dosage and/or an effective amount may vary according to the response of the subject or purpose of treatment. The practitioner responsible for administration may, in any event, determine the concentration of active ingredient(s) in a composition and appropriate dose(s) for the individual subject.

In certain embodiments, pharmaceutical compositions may include, for example, at least about 0.1% of an active compound. In other embodiments, an active compound may comprise between about 2% to about 75% of the weight of the unit, or between about 25% to about 60%, for example, and any range derivable therein. Naturally, the amount of active ingredient in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one of ordinary skill in the art of preparing such pharmaceutical formulations, and as such, a variety of dosages and treatment regimens may be desirable.

For parenteral administration in an aqueous solution, for example, a solution including an active ingredient may be suitably buffered if necessary and a liquid diluent first rendered isotonic with sufficient saline or glucose. These particular aqueous solutions are especially suitable for intravenous, intramuscular, subcutaneous, and intraperitoneal administration. In this connection, sterile aqueous media that may be employed. For example, one dosage may be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety and purity standards as required by FDA Office of Biologics standards.

Sterile injectable solutions may be prepared by incorporating an active ingredient in a solvent with various other ingredients enumerated above, followed by filtered sterilization. Generally, dispersions may be prepared by incorporating various sterilized active ingredients into a sterile vehicle which contains the basic dispersion medium and the required other ingredients from those enumerated above. In the case of sterile powders for the preparation of sterile injectable solutions, preferred methods of preparation include vacuum-drying and freeze-drying techniques which yield a powder of the active ingredient plus any additional desired ingredient from a previously sterile-filtered solution thereof. A powdered composition may be combined with a liquid carrier such as, e.g., water or a saline solution, with or without a stabilizing agent.

Examples of amino acid sequences encoding examples of CaMK and of CREB disclosed herein are presented in Table I, with examples of nucleotide sequences encoding therefor. As would be appreciated, owing to codon degeneracy, amino acid sequences presented in Table I may be encoded for by nucleotide sequences other than those presented in Table 2, and all such variations of possible nucleotide sequences encoding the amino acid sequences presented in Table 2 are explicitly included herein. N-terminal catalytic domains of CaMK proteins lack regulatory regions of full CaMK proteins (located C-terminal to the catalytic N-terminal domains), freeing the catalytic N-terminal domains from inhibitory control otherwise exerted thereon by regulatory regions.

TABLE 2
amino acid and nucleotide sequences

| SEQ ID NO: | Product | Sequence |
|---|---|---|
| 10 | VP16-CREB | ATGGGAGCCCGGGAGATCTGGATCTGGGCCCCC |
| | nucleotide | CCGACCGATGTCAGCCTGGGGGACGAGCTCCAC |
| | | TTAGACGGCGAGGACGTGGCGATGGCGCATGCC |
| | | GACGCGCTAGACGATTTCGATCTGGACATGTTG |
| | | GGGGACGGGGATTCCCCGGGTCCGGGATTTACC |
| | | CCCCACGACTCCGCCCCCTACGGCGCTCTGGATG |
| | | TGGCCGACTTCGAGTTTGAGCAGATGTTTACCGA |
| | | TGCCCTTGGCATTGACGACTTTGGGGGGGGGCG |
| | | CGCTACACAGCCTGCTGAAGAAGCAGCACGAAA |
| | | GAGAGAGGTTCGTCTAATGAAGAACAGGGAAGC |
| | | AGCAAGAGAATGTCGTAGAAAGAAGAAAGAAT |
| | | ATGTGAAATGTTTAGAGAACAGAGTGGCAGTGC |
| | | TTGAAAACCAAAACAAAACATTGATTGAGGAGC |
| | | TAAAAGCACTTAAGGACCTTTACTGCCACAAGT |
| | | CAGATTAA |
| 11 | VP16-CREB | MGAREIWIWAPPTDVSLGDELHLDGEDVAMAHA |
| | amino acid | DALDDFDLDMLGDGDSPGPGFTPHDSAPYGALDV |
| | | ADFEFEQMFTDALGIDDFGGGRATQPAEEAARKR |
| | | EVRLMKNREAARECRRKKKEYVKCLENRVAVLE |
| | | NQNKTLIEELKALKDLYCHKSD |
| 12 | MOUSE CaMKIV | ATGCTCAAAGTCACGGTGCCCTCCTGTCCCTCCT |
| | N-terminal catalytic | CGCCCTGCTCCTCGGTCACCGCCAGTACTGAGAA |
| | domain | CCTCGTCCCGGATTACTGGATCGACGGCTCTAAC |
| | nucleotide | CGAGATCCTCTGGGCGATTTCTTCGAGGTGGAGT |
| | | CAGAGCTGGGACGGGGTGCTACATCCATTGTGT |
| | | ACAGATGCAAACAGAAGGGGACCCAGAAGCCCT |
| | | ATGCTCTCAAAGTGTTAAAGAAAACAGTGGACA |
| | | AGAAGATTGTGAGAACAGAAATAGGAGTTCTCC |
| | | TGCGTCTCTCACACCCGAACATCATAAAACTCAA |
| | | GGAAATATTCGAAACCCCCACAGAAATCAGCCT |
| | | GGTCCTTGAGCTGGTCACAGGAGGAGAACTGTT |
| | | TGACAGGATTGTGGAGAAGGGATACTACAGTGA |
| | | GCGCGATGCGGCTGACGCGGTGAAGCAGATCCT |
| | | GGAGGCCGTTGCTTACCTGCATGAAAATGGGAT |
| | | TGTCCATCGTGACCTCAAACCAGAGAATCTTCTT |
| | | TATGCAACTCCAGCCCCTGATGCACCCCTCAAAA |
| | | TTGCTGATTTTGGACTTTCAAAAATTGTGGAACA |
| | | TCAAGTGCTCATGAAGACAGTGTGTGGAACCCC |
| | | GGGGTACTGCGCACCTGAGATTCTCCGAGGCTG |
| | | TGCCTACGGACCTGAGGTGGACATGTGGTCTGT |
| | | AGGAATAATCACCTACATCCTACTTTGTGGATTT |
| | | GAACCATTCTATGACGAGCGAGGTGATCAGTTC |
| | | ATGTTCAGGAGAATTCTGAATTGTGAATATTACT |
| | | TTATCTCCCCCTGGTGGGATGAAGTGTCTTTAAA |
| | | TGCCAAGGACTTGGTCAAAAAGCTCATTGTTTTG |
| | | GATCCCAAGAAACGGCTGACTACATTTCAAGCC |
| | | CTCCAACACCCATGGGTCACAGGTAAAGCGGCC |
| | | AACTTTGTTCACATGGACACTGCTCAGAAGAAA |
| | | CTTTAA |
| 13 | MOUSE CaMKIV | MLKVTVPSCPSSPCSSVTASTENLVPDYWIDGSNR |
| | N-terminal catalytic | DPLGDFFEVESELGRGATSIVYRCKQKGTQKPYAL |
| | domain | KVLKKTVDKKIVRTEIGVLLRLSHPNIIKLKEIFETP |
| | amino acid | TEISLVLELVTGGELFDRIVEKGYYSERDAADAVK |
| | | QILEAVAYLHENGIVHRDLKPENLLYATPAPDAPL |
| | | KIADFGLSKIVEHQVLMKTVCGTPGYCAPEILRGC |
| | | AYGPEVDMWSVGIITYILLCGFEPFYDERGDQFMF |
| | | RRILNCEYYFISPWWDEVSLNAKDLVKKLIVLDPK |
| | | KRLTTFQALQHPWVTGKAANFVHMDTAQKKL |
| 14 | MOUSE CaMKIV | ATGCTCAAAGTCACGGTGCCCTCCTGTCCCTCCT |
| | nucleotide | CGCCCTGCTCCTCGGTCACCGCCAGTACTGAGAA |
| | | CCTCGTCCCGGATTACTGGATCGACGGCTCTAAC |
| | | CGAGATCCTCTGGGCGATTTCTTCGAGGTGGAGT |
| | | CAGAGCTGGGACGGGGTGCTACATCCATTGTGT |
| | | ACAGATGCAAACAGAAGGGGACCCAGAAGCCCT |
| | | ATGCTCTCAAAGTGTTAAAGAAAACAGTGGACA |
| | | AGAAGATTGTGAGAACAGAAATAGGAGTTCTCC |
| | | TGCGTCTCTCACACCCGAACATCATAAAACTCAA |
| | | GGAAATATTCGAAACCCCCACAGAAATCAGCCT |
| | | GGTCCTTGAGCTGGTCACAGGAGGAGAACTGTT |
| | | TGACAGGATTGTGGAGAAGGGATACTACAGTGA |
| | | GCGCGATGCGGCTGACGCGGTGAAGCAGATCCT |
| | | GGAGGCCGTTGCTTACCTGCATGAAAATGGGAT |
| | | TGTCCATCGTGACCTCAAACCAGAGAATCTTCTT |
| | | TATGCAACTCCAGCCCCTGATGCACCCCTCAAAA |
| | | TTGCTGATTTTGGACTTTCAAAAATTGTGGAACA |
| | | TCAAGTGCTCATGAAGACAGTGTGTGGAACCCC |
| | | GGGGTACTGCGCACCTGAGATTCTCCGAGGCTG |
| | | TGCCTACGGACCTGAGGTGGACATGTGGTCTGT |
| | | AGGAATAATCACCTACATCCTACTTTGTGGATTT |
| | | GAACCATTCTATGACGAGCGAGGTGATCAGTTC |
| | | ATGTTCAGGAGAATTCTGAATTGTGAATATTACT |
| | | TTATCTCCCCCTGGTGGGATGAAGTGTCTTTAAA |
| | | TGCCAAGGACTTGGTCAAAAAGCTCATTGTTTTG |
| | | GATCCCAAGAAACGGCTGACTACATTTCAAGCC |
| | | CTCCAACACCCATGGGTCACAGGTAAAGCGGCC |
| | | AACTTTGTTCACATGGACACTGCTCAGAAGAAA |
| | | CTTCAAGAATTCAATGCTCGGCGCAAGCTTAAG |
| | | GCAGCGGTGAAGGCTGTGGTGGCCTCTTCTCGG |
| | | CTGGGAAGTGCCAGCAGTAGCCACACCAGCATC |
| | | CAAGAGAACCACAAGGCCAGCTCGGATCCACCT |
| | | TCAACCCAAGATGCCAAGGACAGCACAGATCTT |
| | | CTGGGAAAGAAAATGCAAGAGGAGGACCAAGA |
| | | GGAGGACCAAGTGGAGGCCGAGGCTTCAGCCGA |
| | | TGAGATGAGGAAGCTGCAGTCCGAGGAGGTGGA |
| | | GAAAGATGCAGGTGTAAAAGAGGAGGAGACCT |
| | | CCAGTATGGTGCCTCAGGATCCAGAGGATGAGC |
| | | TGGAAACAGATGACCCAGAGATGAAGAGGGATT |
| | | CAGAGGAGAAGCTGAAGAGTGTGGAGGAAGAA |
| | | ATGGACCCCATGACTGAGGAGGAAGCCCCTGAC |
| | | GCGGGACTTGGGGTTCCACAGCAGGATGCGATT |
| | | CAGCCAGAGTACTAA |
| 15 | MOUSE CaMKIV | MLKVTVPSCPSSPCSSVTASTENLVPDYWIDGSNR |
| | amino acid | DPLGDFFEVESELGRGATSIVYRCKQKGTQKPYAL |
| | | KVLKKTVDKKIVRTEIGVLLRLSHPNIIKLKEIFETP |
| | | TEISLVLELVTGGELFDRIVEKGYYSERDAADAVK |
| | | QILEAVAYLHENGIVHRDLKPENLLYATPAPDAPL |
| | | KIADFGLSKIVEHQVLMKTVCGTPGYCAPEILRGC |
| | | AYGPEVDMWSVGIITYILLCGFEPFYDERGDQFMF |
| | | RRILNCEYYFISPWWDEVSLNAKDLVKKLIVLDPK |
| | | KRLTTFQALQHPWVTGKAANFVHMDTAQKKLQE |
| | | FNARRKLKAAVKAVVASSRLGSASSSHTSIQENHK |
| | | ASSDPPSTQDAKDSTDLLGKKMQEEDQEEDQVEA |
| | | EASADEMRKLQSEEVEKDAGVKEEETSSMVPQDP |
| | | EDELETDDPEMKRDSEEKLKSVEEEMDPMTEEEAP |
| | | DAGLGVPQQDAIQPEY |
| 16 | HUMAN CaMKIV | ATGCTCAAAGTCACGGTGCCCTCCTGCTCCGCCT |
| | N-terminal catalytic | CGTCCTGCTCTTCGGTCACCGCCAGTGCGGCCCC |
| | domain | GGGGACCGCGAGCCTCGTCCCGGATTACTGGAT |
| | nucleotide | CGACGGCTCCAACAGGGATGCGCTGAGCGATTT |
| | | CTTCGAGGTGGAGTCGGAGCTGGGACGGGGTGC |
| | | TACATCCATTGTGTACAGATGCAAACAGAAGGG |
| | | GACCCAGAAGCCTTATGCTCTCAAAGTGTTAAA |
| | | GAAAACAGTGGACAAAAAAATCGTAAGAACTG |
| | | AGATAGGAGTTCTTCTTCGCCTCTCACATCCAAA |
| | | CATTATAAAACTTAAAGAGATATTTGAAACCCCT |
| | | ACAGAAATCAGTCTGGTCCTAGAACTCGTCACA |
| | | GGAGGAGAACTGTTTGATAGGATTGTGGAAAAG |
| | | GGATATTACAGTGAGCGAGATGCTGCAGATGCC |
| | | GTTAAACAAATCCTGGAGGCAGTTGCTTATCTAC |
| | | ATGAAAATGGGATTGTCCATCGTGATCTCAAAC |
| | | CAGAGAATCTTCTTTATGCAACTCCAGCCCCAGA |
| | | TGCACCACTCAAAATCGCTGATTTTGGACTCTCT |
| | | AAAATTGTGGAACATCAAGTGCTCATGAAGACA |
| | | GTATGTGGAACCCCAGGGTACTGCGCACCTGAA |
| | | ATTCTTAGAGGTTGTGCCTATGGACCTGAGGTGG |
| | | ACATGTGGTCTGTAGGAATAATCACCTACATCTT |
| | | ACTTTGTGGATTTGAACCATTCTATGATGAAAGA |
| | | GGCGATCAGTTCATGTTCAGGAGAATTCTGAATT |
| | | GTGAATATTACTTTATCTCCCCCTGGTGGGATGA |
| | | AGTATCTCTAAATGCCAAGGACTTGGTCAGAAA |
| | | ATTAATTGTTTTGGATCCAAAGAAACGGCTGACT |
| | | ACATTTCAAGCTCTCCAGCATCCGTGGGTCACAG |
| | | GTAAAGCAGCCAATTTTGTACACATGGATACCG |
| | | CTCAAAAGAAGCTCTAA |
| 17 | HUMAN CaMKIV | MLKVTVPSCSASSCSSVTASAAPGTASLVPDYWID |
| | N-terminal catalytic | GSNRDALSDFFEVESELGRGATSIVYRCKQKGTQK |
| | domain | PYALKVLKKTVDKKIVRTEIGVLLRLSHPNIIKLKEI |
| | amino acid | FETPTEISLVLELVTGGELFDRIVEKGYYSERDAAD |
| | | AVKQILEAVAYLHENGIVHRDLKPENLLYATPAPD |
| | | APLKIADFGLSKIVEHQVLMKTVCGTPGYCAPEILR |
| | | GCAYGPEVDMWSVGIITYILLCGFEPFYDERGDQF |
| | | MFRRILNCEYYFISPWWDEVSLNAKDLVRKLIVLD |
| | | PKKRLTTFQALQHPWVTGKAANFVHMDTAQKKL |
| 18 | HUMAN CaMKIV | ATGCTCAAAGTCACGGTGCCCTCCTGCTCCGCCT |
| | nucleotide | CGTCCTGCTCTTCGGTCACCGCCAGTGCGGCCCC |
| | | GGGGACCGCGAGCCTCGTCCCGGATTACTGGAT |
| | | CGACGGCTCCAACAGGGATGCGCTGAGCGATTT |
| | | CTTCGAGGTGGAGTCGGAGCTGGGACGGGGTGC |
| | | TACATCCATTGTGTACAGATGCAAACAGAAGGG |
| | | GACCCAGAAGCCTTATGCTCTCAAAGTGTTAAA |
| | | GAAAACAGTGGACAAAAAAATCGTAAGAACTG |
| | | AGATAGGAGTTCTTCTTCGCCTCTCACATCCAAA |
| | | CATTATAAAACTTAAAGAGATATTTGAAACCCCT |
| | | ACAGAAATCAGTCTGGTCCTAGAACTCGTCACA |
| | | GGAGGAGAACTGTTTGATAGGATTGTGGAAAAG |
| | | GGATATTACAGTGAGCGAGATGCTGCAGATGCC |
| | | GTTAAACAAATCCTGGAGGCAGTTGCTTATCTAC |
| | | ATGAAAATGGGATTGTCCATCGTGATCTCAAAC |
| | | CAGAGAATCTTCTTTATGCAACTCCAGCCCCAGA |
| | | TGCACCACTCAAAATCGCTGATTTTGGACTCTCT |
| | | AAAATTGTGGAACATCAAGTGCTCATGAAGACA |
| | | GTATGTGGAACCCCAGGGTACTGCGCACCTGAA |
| | | ATTCTTAGAGGTTGTGCCTATGGACCTGAGGTGG |
| | | ACATGTGGTCTGTAGGAATAATCACCTACATCTT |
| | | ACTTTGTGGATTTGAACCATTCTATGATGAAAGA |
| | | GGCGATCAGTTCATGTTCAGGAGAATTCTGAATT |
| | | GTGAATATTACTTTATCTCCCCCTGGTGGGATGA |
| | | AGTATCTCTAAATGCCAAGGACTTGGTCAGAAA |
| | | ATTAATTGTTTTGGATCCAAAGAAACGGCTGACT |
| | | ACATTTCAAGCTCTCCAGCATCCGTGGGTCACAG |
| | | GTAAAGCAGCCAATTTTGTACACATGGATACCG |
| | | CTCAAAAGAAGCTCCAAGAATTCAATGCCCGGC |
| | | GTAAGCTTAAGGCAGCGGTGAAGGCTGTGGTGG |
| | | CCTCTTCGCGCCTGGGAAGTGCCAGCAGCAGCC |
| | | ATGGCAGCATCCAGGAGAGCCACAAGGCTAGCC |
| | | GAGACCCTTCTCCAATCCAAGATGGCAACGAGG |
| | | ACATGAAAGCTATTCCAGAAGGAGAGAAAATTC |
| | | AAGGCGATGGGGCCCAAGCCGCAGTTAAGGGGG |
| | | CACAGGCTGAGCTGATGAAGGTGCAAGCCTTAG |
| | | AGAAAGTTAAAGGTGCAGATATAAATGCTGAAG |
| | | AGGCCCCCAAAATGGTGCCCAAGGCAGTGGAGG |
| | | ATGGGATAAAGGTGGCTGACCTGGAACTAGAGG |
| | | AGGGCCTAGCAGAGGAGAAGCTGAAGACTGTGG |
| | | AGGAGGCAGCAGCTCCCAGAGAAGGGCAAGGA |
| | | AGCTCTGCTGTGGGTTTTGAAGTTCCACAGCAAG |
| | | ATGTGATCCTGCCAGAGTACTAA |
| 19 | HUMAN CaMKIV | MLKVTVPSCSASSCSSVTASAAPGTASLVPDYWID |
| | amino acid | GSNRDALSDFFEVESELGRGATSIVYRCKQKGTQK |
| | | PYALKVLKKTVDKKIVRTEIGVLLRLSHPNIIKLKEI |
| | | FETPTEISLVLELVTGGELFDRIVEKGYYSERDAAD |
| | | AVKQILEAVAYLHENGIVHRDLKPENLLYATPAPD |
| | | APLKIADFGLSKIVEHQVLMKTVCGTPGYCAPEILR |
| | | GCAYGPEVDMWSVGIITYILLCGFEPFYDERGDQF |
| | | MFRRILNCEYYFISPWWDEVSLNAKDLVRKLIVLD |
| | | PKKRLTTFQALQHPWVTGKAANFVHMDTAQKKL |
| | | QEFNARRKLKAAVKAVVASSRLGSASSSHGSIQES |
| | | HKASRDPSPIQDGNEDMKAIPEGEKIQGDGAQAAV |
| | | KGAQAELMKVQALEKVKGADINAEEAPKMVPKA |
| | | VEDGIKVADLELEEGLAEEKLKTVEEAAAPREGQG |
| | | SSAVGFEVPQQDVILPEY |
| 20 | RAT CaMKIIβ | ATGGCCACCACGGTGACCTGCACCCGTTTCACG |
| | N-terminal catalytic | GACGAGTACCAGCTATACGAGGATATTGGCAAG |
| | domain | GGGGCTTTCTCTGTGGTCCGACGCTGTGTCAAGC |
| | nucleotide | TCTGCACCGGCCATGAGTATGCAGCTAAGATCA |
| | | TTAACACCAAGAAGCTGTCAGCTAGAGATCACC |
| | | AGAAGCTGGAGAGGGAGGCTCGGATCTGCCGCC |
| | | TGCTGAAGCATTCCAACATTGTACGCCTCCATGA |
| | | CAGCATCTCTGAAGAGGGCTTCCACTACCTGGTC |
| | | TTCGACCTGGTCACTGGTGGGGAGCTCTTTGAAG |
| | | ACATTGTGGCGAGAGAGTACTACAGTGAGGCTG |
| | | ACGCCAGTCACTGTATCCAGCAGATCCTGGAGG |
| | | CTGTTCTCCATTGTCACCAAATGGGGGTCGTCCA |
| | | CAGAGACCTCAAGCCTGAAAACCTGCTCCTGGC |
| | | CAGCAAATGCAAAGGGGCCGCAGTGAAACTGGC |
| | | AGACTTCGGCCTGGCCATCGAGGTTCAGGGAGA |
| | | CCAGCAGGCATGGTTTGGATTTGCGGGAACACC |
| | | AGGCTACCTGTCTCCCGAAGTTCTTCGGAAGGA |
| | | GGCCTATGGCAAACCAGTGGATATCTGGGCATG |
| | | TGGGGTGATCCTGTATATCCTGCTGGTGGGATAC |
| | | CCACCTTTCTGGGATGAGGACCAGCACAAGCTG |
| | | TACCAGCAGATCAAGGCTGGGGCCTATGACTTC |
| | | CCATCCCCCGAGTGGGACACCGTTACCCCTGAA |
| | | GCCAAAAACCTCATCAACCAGATGTTGACCATC |
| | | AACCCCGCCAAGCGCATCACGGCCCACGAGGCC |
| | | CTGAAGCACCCATGGGTCTGCCAACGATCCACG |
| | | GTGGCCTCCATGATGCACAGACAGGAGACTGTG |
| | | GAATGTCTGTGA |
| 21 | RAT CaMKIIß | MATTVTCTRFTDEYQLYEDIGKGAFSVVRRCVKL |
| | N-terminal catalytic | CTGHEYAAKIINTKKLSARDHQKLEREARICRLLK |
| | domain | HSNIVRLHDSISEEGFHYLVFDLVTGGELFEDIVAR |
| | amino acid | EYYSEADASHCIQQILEAVLHCHQMGVVHRDLKP |
| | | ENLLLASKCKGAAVKLADFGLAIEVQGDQQAWFG |
| | | FAGTPGYLSPEVLRKEAYGKPVDIWACGVILYILL |
| | | VGYPPFWDEDQHKLYQQIKAGAYDFPSPEWDTVT |
| | | PEAKNLINQMLTINPAKRITAHEALKHPWVCQRST |
| | | VASMMHRQETVECL |
| 22 | RAT CaMKIIß | ATGGCCACCACGGTGACCTGCACCCGTTTCACG |
| | T287D | GACGAGTACCAGCTATACGAGGATATTGGCAAG |
| | nucleotide | GGGGCTTTCTCTGTGGTCCGACGCTGTGTCAAGC |
| | | TCTGCACCGGCCATGAGTATGCAGCTAAGATCA |
| | | TTAACACCAAGAAGCTGTCAGCTAGAGATCACC |
| | | AGAAGCTGGAGAGGGAGGCTCGGATCTGCCGCC |
| | | TGCTGAAGCATTCCAACATTGTACGCCTCCATGA |
| | | CAGCATCTCTGAAGAGGGCTTCCACTACCTGGTC |
| | | TTCGACCTGGTCACTGGTGGGGAGCTCTTTGAAG |
| | | ACATTGTGGCGAGAGAGTACTACAGTGAGGCTG |
| | | ACGCCAGTCACTGTATCCAGCAGATCCTGGAGG |
| | | CTGTTCTCCATTGTCACCAAATGGGGGTCGTCCA |
| | | CAGAGACCTCAAGCCTGAAAACCTGCTCCTGGC |
| | | CAGCAAATGCAAAGGGGCCGCAGTGAAACTGGC |
| | | AGACTTCGGCCTGGCCATCGAGGTTCAGGGAGA |
| | | CCAGCAGGCATGGTTTGGATTTGCGGGAACACC |
| | | AGGCTACCTGTCTCCCGAAGTTCTTCGGAAGGA |
| | | GGCCTATGGCAAACCAGTGGATATCTGGGCATG |
| | | TGGGGTGATCCTGTATATCCTGCTGGTGGGATAC |
| | | CCACCTTTCTGGGATGAGGACCAGCACAAGCTG |
| | | TACCAGCAGATCAAGGCTGGGGCCTATGACTTC |
| | | CCATCCCCCGAGTGGGACACCGTTACCCCTGAA |
| | | GCCAAAAACCTCATCAACCAGATGTTGACCATC |
| | | AACCCCGCCAAGCGCATCACGGCCCACGAGGCC |
| | | CTGAAGCACCCATGGGTCTGCCAACGATCCACG |
| | | GTGGCCTCCATGATGCACAGACAGGAGGATGTG |
| | | GAATGTCTGAAGAAGTTCAATGCAAGGAGGAAG |
| | | CTCAAGGGAGCCATCCTCACCACTATGCTGGCC |
| | | ACACGGAATTTCTCAGTGGGCAGACAGACCACC |
| | | GCTCCGGCCACAATGTCCACCGCGGCCTCCGGC |
| | | ACCACCATGGGGCTGGTGGAACAAGCCAAGAGT |
| | | TTACTCAACAAGAAAGCAGACGGAGTCAAGCCC |
| | | CAGACAAACAGCACCAAAAACAGCTCGGCCATC |
| | | ACCAGCCCCAAAGGATCCCTCCCTCCTGCCGCCC |
| | | TGGAGCCTCAAACCACCGTTATCCATAACCCAGT |
| | | GGACGGCATTAAGGAATCTTCCGACAGCACCAA |
| | | CACAACCATAGAGGACGAAGATGCCAAAGCCCG |
| | | GAAGCAGGAAATCATCAAGACCACAGAGCAGCT |
| | | CATCGAGGCCGTCAACAACGGCGACTTTGAGGC |
| | | CTATGCGAAAATCTGTGACCCAGGCCTGACCTC |
| | | ATTTGAGCCTGAAGCTCTGGGCAACCTGGTCGA |
| | | AGGGATGGATTTCCACAGATTCTACTTTGAGAAC |
| | | CTGCTGGCCAAGAACAGCAAGCCGATCCACACC |
| | | ACTATCCTGAACCCGCACGTGCACGTCATCGGC |
| | | GAGGATGCAGCCTGCATCGCTTACATCCGCCTCA |
| | | CACAGTACATCGACGGCCAGGGCAGACCCCGCA |
| | | CCAGCCAGTCCGAAGAGACCCGTGTGTGGCACC |
| | | GCCGCGACGGCAAGTGGCAGAATGTCCATTTCC |
| | | ACTGCTCGGGCGCTCCAGTGGCCCCACTGCAGT |
| | | GA |
| 23 | RAT CaMKIIß | MATTVTCTRFTDEYQLYEDIGKGAFSVVRRCVKL |
| | T287D | CTGHEYAAKIINTKKLSARDHQKLEREARICRLLK |
| | amino acid | HSNIVRLHDSISEEGFHYLVFDLVTGGELFEDIVAR |
| | | EYYSEADASHCIQQILEAVLHCHQMGVVHRDLKP |
| | | ENLLLASKCKGAAVKLADFGLAIEVQGDQQAWFG |
| | | FAGTPGYLSPEVLRKEAYGKPVDIWACGVILYILL |
| | | VGYPPFWDEDQHKLYQQIKAGAYDFPSPEWDTVT |
| | | PEAKNLINQMLTINPAKRITAHEALKHPWVCQRST |
| | | VASMMHRQEDVECLKKFNARRKLKGAILTTMLAT |
| | | RNFSVGRQTTAPATMSTAASGTTMGLVEQAKSLL |
| | | NKKADGVKPQTNSTKNSSAITSPKGSLPPAALEPQ |
| | | TTVIHNPVDGIKESSDSTNTTIEDEDAKARKQEIIKT |
| | | TEQLIEAVNNGDFEAYAKICDPGLTSFEPEALGNLV |
| | | EGMDFHRFYFENLLAKNSKPIHTTILNPHVHVIGED |
| | | AACIAYIRLTQYIDGQGRPRTSQSEETRVWHRRDG |
| | | KWQNVHFHCSGAPVAPLQ |
| 24 | RAT CaMKIIß | ATGGCCACCACGGTGACCTGCACCCGTTTCACG |
| | nucleotide | GACGAGTACCAGCTATACGAGGATATTGGCAAG |
| | | GGGGCTTTCTCTGTGGTCCGACGCTGTGTCAAGC |
| | | TCTGCACCGGCCATGAGTATGCAGCTAAGATCA |
| | | TTAACACCAAGAAGCTGTCAGCTAGAGATCACC |
| | | AGAAGCTGGAGAGGGAGGCTCGGATCTGCCGCC |
| | | TGCTGAAGCATTCCAACATTGTACGCCTCCATGA |
| | | CAGCATCTCTGAAGAGGGCTTCCACTACCTGGTC |
| | | TTCGACCTGGTCACTGGTGGGGAGCTCTTTGAAG |
| | | ACATTGTGGCGAGAGAGTACTACAGTGAGGCTG |
| | | ACGCCAGTCACTGTATCCAGCAGATCCTGGAGG |
| | | CTGTTCTCCATTGTCACCAAATGGGGGTCGTCCA |
| | | CAGAGACCTCAAGCCTGAAAACCTGCTCCTGGC |
| | | CAGCAAATGCAAAGGGGCCGCAGTGAAACTGGC |
| | | AGACTTCGGCCTGGCCATCGAGGTTCAGGGAGA |
| | | CCAGCAGGCATGGTTTGGATTTGCGGGAACACC |
| | | AGGCTACCTGTCTCCCGAAGTTCTTCGGAAGGA |
| | | GGCCTATGGCAAACCAGTGGATATCTGGGCATG |
| | | TGGGGTGATCCTGTATATCCTGCTGGTGGGATAC |
| | | CCACCTTTCTGGGATGAGGACCAGCACAAGCTG |
| | | TACCAGCAGATCAAGGCTGGGGCCTATGACTTC |
| | | CCATCCCCCGAGTGGGACACCGTTACCCCTGAA |
| | | GCCAAAAACCTCATCAACCAGATGTTGACCATC |
| | | AACCCCGCCAAGCGCATCACGGCCCACGAGGCC |
| | | CTGAAGCACCCATGGGTCTGCCAACGATCCACG |
| | | GTGGCCTCCATGATGCACAGACAGGAGACTGTG |
| | | GAATGTCTGAAGAAGTTCAATGCAAGGAGGAAG |
| | | CTCAAGGGAGCCATCCTCACCACTATGCTGGCC |
| | | ACACGGAATTTCTCAGTGGGCAGACAGACCACC |
| | | GCTCCGGCCACAATGTCCACCGCGGCCTCCGGC |
| | | ACCACCATGGGGCTGGTGGAACAAGCCAAGAGT |
| | | TTACTCAACAAGAAAGCAGACGGAGTCAAGCCC |
| | | CAGACAAACAGCACCAAAAACAGCTCGGCCATC |
| | | ACCAGCCCCAAAGGATCCCTCCCTCCTGCCGCCC |
| | | TGGAGCCTCAAACCACCGTTATCCATAACCCAGT |
| | | GGACGGCATTAAGGAATCTTCCGACAGCACCAA |
| | | CACAACCATAGAGGACGAAGATGCCAAAGCCCG |
| | | GAAGCAGGAAATCATCAAGACCACAGAGCAGCT |
| | | CATCGAGGCCGTCAACAACGGCGACTTTGAGGC |
| | | CTATGCGAAAATCTGTGACCCAGGCCTGACCTC |
| | | ATTTGAGCCTGAAGCTCTGGGCAACCTGGTCGA |
| | | AGGGATGGATTTCCACAGATTCTACTTTGAGAAC |
| | | CTGCTGGCCAAGAACAGCAAGCCGATCCACACC |
| | | ACTATCCTGAACCCGCACGTGCACGTCATCGGC |
| | | GAGGATGCAGCCTGCATCGCTTACATCCGCCTCA |
| | | CACAGTACATCGACGGCCAGGGCAGACCCCGCA |
| | | CCAGCCAGTCCGAAGAGACCCGTGTGTGGCACC |
| | | GCCGCGACGGCAAGTGGCAGAATGTCCATTTCC |
| | | ACTGCTCGGGCGCTCCAGTGGCCCCACTGCAGT |
| | | GA |
| 25 | RAT CaMKIIß | MATTVTCTRFTDEYQLYEDIGKGAFSVVRRCVKL |
| | amino acid | CTGHEYAAKIINTKKLSARDHQKLEREARICRLLK |
| | | HSNIVRLHDSISEEGFHYLVFDLVTGGELFEDIVAR |
| | | EYYSEADASHCIQQILEAVLHCHQMGVVHRDLKP |
| | | ENLLLASKCKGAAVKLADFGLAIEVQGDQQAWFG |
| | | FAGTPGYLSPEVLRKEAYGKPVDIWACGVILYILL |
| | | VGYPPFWDEDQHKLYQQIKAGAYDFPSPEWDTVT |
| | | PEAKNLINQMLTINPAKRITAHEALKHPWVCQRST |
| | | VASMMHRQETVECLKKFNARRKLKGAILTTMLAT |
| | | RNFSVGRQTTAPATMSTAASGTTMGLVEQAKSLL |
| | | NKKADGVKPQTNSTKNSSAITSPKGSLPPAALEPQ |
| | | TTVIHNPVDGIKESSDSTNTTIEDEDAKARKQEIIKT |
| | | TEQLIEAVNNGDFEAYAKICDPGLTSFEPEALGNLV |
| | | EGMDFHRFYFENLLAKNSKPIHTTILNPHVHVIGED |
| | | AACIAYIRLTQYIDGQGRPRTSQSEETRVWHRRDG |
| | | KWQNVHFHCSGAPVAPLQ |
| 26 | RAT CaMKIIα | ATGGCTACCATCACCTGCACCCGATTCACGGAA |
| | T286D | GAGTACCAGCTCTTCGAGGAACTGGGAAAGGGA |
| | nucleotide | GCCTTCTCCGTGGTGCGCAGGTGTGTGAAGGTGC |
| | | TGGCTGGCCAGGAGTATGCTGCCAAGATTATCA |
| | | ACACCAAGAAGCTCTCAGCCAGAGATCACCAGA |
| | | AGTTGGAACGCGAGGCCCGCATCTGCCGCTTGTT |
| | | GAAGCACCCCAATATCGTCCGACTCCATGACAG |
| | | CATCTCCGAGGAGGGGCACCACTACCTTATCTTC |
| | | GATCTGGTCACTGGTGGGGAGCTGTTCGAAGAC |
| | | ATTGTGGCCCGGGAGTATTACAGTGAGGCTGAT |
| | | GCCAGCCACTGTATCCAGCAGATCCTGGAGGCT |
| | | GTGCTACACTGTCACCAGATGGGGGTGGTGCAT |
| | | CGCGACCTGAAGCCTGAGAATCTGTTGCTGGCTT |
| | | CGAAGCTCAAGGGTGCTGCGGTGAAGCTGGCAG |
| | | ACTTTGGCCTGGCCATAGAGGTTGAGGGAGAGC |
| | | AGCAGGCATGGTTTGGGTTCGCAGGGACACCTG |
| | | GATACCTCTCCCCAGAAGTGCTGCGGAAGGACC |
| | | CATACGGGAAGCCTGTGGACCTGTGGGCCTGTG |
| | | GCGTCATCCTGTATATCTTGCTGGTTGGGTATCC |
| | | CCCATTCTGGGATGAGGACCAGCACCGCCTGTA |
| | | CCAGCAGATCAAAGCTGGTGCCTACGATTTCCC |
| | | ATCACCAGAATGGGACACCGTCACCCCGGAAGC |
| | | CAAGGATCTGATCAATAAGATGCTGACCATCAA |
| | | CCCGTCCAAACGCATCACGGCCGCTGAGGCTCT |
| | | CAAGCACCCCTGGATCTCGCACCGCTCCACTGTG |
| | | GCCTCCTGCATGCACAGACAGGAGGACGTGGAC |
| | | TGCCTGAAGAAGTTCAATGCCAGGAGGAAACTG |
| | | AAGGGAGCCATCCTCACCACTATGCTGGCCACC |
| | | AGGAACTTCTCCGGAGGGAAGAGTGGAGGAAAC |
| | | AAGAAGAATGATGGCGTGAAGGAATCCTCTGAG |
| | | AGCACCAACACCACCATCGAGGATGAAGACACC |
| | | AAAGTGCGCAAACAGGAAATTATCAAAGTGACA |
| | | GAGCAGCTGATCGAAGCCATAAGCAATGGAGAC |
| | | TTTGAATCCTACACGAAGATGTGCGACCCTGGA |
| | | ATGACAGCCTTTGAACCGGAGGCCCTGGGGAAC |
| | | CTGGTCGAGGGCCTGGACTTTCATCGATTCTATT |
| | | TTGAAAACCTGTGGTCCCGGAACAGCAAGCCCG |
| | | TGCACACCACCATCCTGAACCCTCACATCCACCT |
| | | GATGGGTGACGAGTCAGCCTGCATCGCCTACAT |
| | | CCGCATCACTCAGTACCTGGATGCGGGTGGCAT |
| | | CCCCCGCACGGCCCAGTCAGAGGAGACCCGTGT |
| | | CTGGCACCGCAGGGATGGAAAATGGCAGATCGT |
| | | CCACTTCCACAGATCTGGGGCGCCCTCCGTCCTG |
| | | CCCCATTGA |
| 27 | RAT CaMKIIα | MATITCTRFTEEYQLFEELGKGAFSVVRRCVKVLA |
| | T286D | GQEYAAKIINTKKLSARDHQKLEREARICRLLKHP |
| | amino acid | NIVRLHDSISEEGHHYLIFDLVTGGELFEDIVAREY |
| | | YSEADASHCIQQILEAVLHCHQMGVVHRDLKPEN |
| | | LLLASKLKGAAVKLADFGLAIEVEGEQQAWFGFA |
| | | GTPGYLSPEVLRKDPYGKPVDLWACGVILYILLVG |
| | | YPPFWDEDQHRLYQQIKAGAYDFPSPEWDTVTPE |
| | | AKDLINKMLTINPSKRITAAEALKHPWISHRSTVAS |
| | | CMHRQEDVDCLKKFNARRKLKGAILTTMLATRNF |
| | | SGGKSGGNKKNDGVKESSESTNTTIEDEDTKVRKQ |
| | | EIIKVTEQLIEAISNGDFESYTKMCDPGMTAFEPEA |
| | | LGNLVEGLDFHRFYFENLWSRNSKPVHTTILNPHI |
| | | HLMGDESACIAYIRITQYLDAGGIPRTAQSEETRV |
| | | WHRRDGKWQIVHFHRSGAPSVLPH |
| 28 | RAT CaMKIIα | ATGGCTACCATCACCTGCACCCGATTCACGGAA |
| | nucleotide | GAGTACCAGCTCTTCGAGGAACTGGGAAAGGGA |
| | | GCCTTCTCCGTGGTGCGCAGGTGTGTGAAGGTGC |
| | | TGGCTGGCCAGGAGTATGCTGCCAAGATTATCA |
| | | ACACCAAGAAGCTCTCAGCCAGAGATCACCAGA |
| | | AGTTGGAACGCGAGGCCCGCATCTGCCGCTTGTT |
| | | GAAGCACCCCAATATCGTCCGACTCCATGACAG |
| | | CATCTCCGAGGAGGGGCACCACTACCTTATCTTC |
| | | GATCTGGTCACTGGTGGGGAGCTGTTCGAAGAC |
| | | ATTGTGGCCCGGGAGTATTACAGTGAGGCTGAT |
| | | GCCAGCCACTGTATCCAGCAGATCCTGGAGGCT |
| | | GTGCTACACTGTCACCAGATGGGGGTGGTGCAT |
| | | CGCGACCTGAAGCCTGAGAATCTGTTGCTGGCTT |
| | | CGAAGCTCAAGGGTGCTGCGGTGAAGCTGGCAG |
| | | ACTTTGGCCTGGCCATAGAGGTTGAGGGAGAGC |
| | | AGCAGGCATGGTTTGGGTTCGCAGGGACACCTG |
| | | GATACCTCTCCCCAGAAGTGCTGCGGAAGGACC |
| | | CATACGGGAAGCCTGTGGACCTGTGGGCCTGTG |
| | | GCGTCATCCTGTATATCTTGCTGGTTGGGTATCC |
| | | CCCATTCTGGGATGAGGACCAGCACCGCCTGTA |
| | | CCAGCAGATCAAAGCTGGTGCCTACGATTTCCC |
| | | ATCACCAGAATGGGACACCGTCACCCCGGAAGC |
| | | CAAGGATCTGATCAATAAGATGCTGACCATCAA |
| | | CCCGTCCAAACGCATCACGGCCGCTGAGGCTCT |
| | | CAAGCACCCCTGGATCTCGCACCGCTCCACTGTG |
| | | GCCTCCTGCATGCACAGACAGGAGACCGTGGAC |
| | | TGCCTGAAGAAGTTCAATGCCAGGAGGAAACTG |
| | | AAGGGAGCCATCCTCACCACTATGCTGGCCACC |
| | | AGGAACTTCTCCGGAGGGAAGAGTGGAGGAAAC |
| | | AAGAAGAATGATGGCGTGAAGGAATCCTCTGAG |
| | | AGCACCAACACCACCATCGAGGATGAAGACACC |
| | | AAAGTGCGCAAACAGGAAATTATCAAAGTGACA |
| | | GAGCAGCTGATCGAAGCCATAAGCAATGGAGAC |
| | | TTTGAATCCTACACGAAGATGTGCGACCCTGGA |
| | | ATGACAGCCTTTGAACCGGAGGCCCTGGGGAAC |
| | | CTGGTCGAGGGCCTGGACTTTCATCGATTCTATT |
| | | TTGAAAACCTGTGGTCCCGGAACAGCAAGCCCG |
| | | TGCACACCACCATCCTGAACCCTCACATCCACCT |
| | | GATGGGTGACGAGTCAGCCTGCATCGCCTACAT |
| | | CCGCATCACTCAGTACCTGGATGCGGGTGGCAT |
| | | CCCCCGCACGGCCCAGTCAGAGGAGACCCGTGT |
| | | CTGGCACCGCAGGGATGGAAAATGGCAGATCGT |
| | | CCACTTCCACAGATCTGGGGCGCCCTCCGTCCTG |
| | | CCCCATTGA |
| 29 | RAT CaMKIIα | MATITCTRFTEEYQLFEELGKGAFSVVRRCVKVLA |
| | amino acid | GQEYAAKIINTKKLSARDHQKLEREARICRLLKHP |
| | | NIVRLHDSISEEGHHYLIFDLVTGGELFEDIVAREY |
| | | YSEADASHCIQQILEAVLHCHQMGVVHRDLKPEN |
| | | LLLASKLKGAAVKLADFGLAIEVEGEQQAWFGFA |
| | | GTPGYLSPEVLRKDPYGKPVDLWACGVILYILLVG |
| | | YPPFWDEDQHRLYQQIKAGAYDFPSPEWDTVTPE |
| | | AKDLINKMLTINPSKRITAAEALKHPWISHRSTVAS |
| | | CMHRQETVDCLKKFNARRKLKGAILTTMLATRNF |
| | | SGGKSGGNKKNDGVKESSESTNTTIEDEDTKVRKQ |
| | | EIIKVTEQLIEAISNGDFESYTKMCDPGMTAFEPEA |
| | | LGNLVEGLDFHRFYFENLWSRNSKPVHTTILNPHI |
| | | HLMGDESACIAYIRITQYLDAGGIPRTAQSEETRV |
| | | WHRRDGKWQIVHFHRSGAPSVLPH |
| 30 | RAT CaMKIIα | ATGGCTACCATCACCTGCACCCGATTCACGGAA |
| | N-terminal catalytic | GAGTACCAGCTCTTCGAGGAACTGGGAAAGGGA |
| | domain nucleotide | GCCTTCTCCGTGGTGCGCAGGTGTGTGAAGGTGC |
| | | TGGCTGGCCAGGAGTATGCTGCCAAGATTATCA |
| | | ACACCAAGAAGCTCTCAGCCAGAGATCACCAGA |
| | | AGTTGGAACGCGAGGCCCGCATCTGCCGCTTGTT |
| | | GAAGCACCCCAATATCGTCCGACTCCATGACAG |
| | | CATCTCCGAGGAGGGGCACCACTACCTTATCTTC |
| | | GATCTGGTCACTGGTGGGGAGCTGTTCGAAGAC |
| | | ATTGTGGCCCGGGAGTATTACAGTGAGGCTGAT |
| | | GCCAGCCACTGTATCCAGCAGATCCTGGAGGCT |
| | | GTGCTACACTGTCACCAGATGGGGGTGGTGCAT |
| | | CGCGACCTGAAGCCTGAGAATCTGTTGCTGGCTT |
| | | CGAAGCTCAAGGGTGCTGCGGTGAAGCTGGCAG |
| | | ACTTTGGCCTGGCCATAGAGGTTGAGGGAGAGC |
| | | AGCAGGCATGGTTTGGGTTCGCAGGGACACCTG |
| | | GATACCTCTCCCCAGAAGTGCTGCGGAAGGACC |
| | | CATACGGGAAGCCTGTGGACCTGTGGGCCTGTG |
| | | GCGTCATCCTGTATATCTTGCTGGTTGGGTATCC |
| | | CCCATTCTGGGATGAGGACCAGCACCGCCTGTA |
| | | CCAGCAGATCAAAGCTGGTGCCTACGATTTCCC |
| | | ATCACCAGAATGGGACACCGTCACCCCGGAAGC |
| | | CAAGGATCTGATCAATAAGATGCTGACCATCAA |
| | | CCCGTCCAAACGCATCACGGCCGCTGAGGCTCT |
| | | CAAGCACCCCTGGATCTCGCACCGCTCCACTGTG |
| | | GCCTCCTGCATGCACAGACAGGAGACCGTGGAC |
| | | TGCCTGTGA |
| 31 | RAT CaMKIIα | MATITCTRFTEEYQLFEELGKGAFSVVRRCVKVLA |
| | N-terminal catalytic | GQEYAAKIINTKKLSARDHQKLEREARICRLLKHP |
| | domain | NIVRLHDSISEEGHHYLIFDLVTGGELFEDIVAREY |
| | amino acid | YSEADASHCIQQILEAVLHCHQMGVVHRDLKPEN |
| | | LLLASKLKGAAVKLADFGLAIEVEGEQQAWFGFA |
| | | GTPGYLSPEVLRKDPYGKPVDLWACGVILYILLVG |
| | | YPPFWDEDQHRLYQQIKAGAYDFPSPEWDTVTPE |
| | | AKDLINKMLTINPSKRITAAEALKHPWISHRSTVAS |
| | | CMHRQETVDCL |
| 32 | HUMAN CaMKIIß | ATGGCCACCACGGTGACCTGCACCCGCTTCACC |
| | T287D | GACGAGTACCAGCTCTACGAGGATATTGGCAAG |
| | nucleotide | GGGGCTTTCTCTGTGGTCCGACGCTGTGTCAAGC |
| | | TCTGCACCGGCCATGAGTATGCAGCCAAGATCA |
| | | TCAACACCAAGAAGCTGTCAGCCAGAGATCACC |
| | | AGAAGCTGGAGAGAGAGGCTCGGATCTGCCGCC |
| | | TTCTGAAGCATTCCAACATCGTGCGTCTCCACGA |
| | | CAGCATCTCCGAGGAGGGCTTCCACTACCTGGTC |
| | | TTCGATCTGGTCACTGGTGGGGAGCTCTTTGAAG |
| | | ACATTGTGGCGAGAGAGTACTACAGCGAGGCTG |
| | | ATGCCAGTCACTGTATCCAGCAGATCCTGGAGG |
| | | CCGTTCTCCATTGTCACCAAATGGGGGTCGTCCA |
| | | CAGAGACCTCAAGCCGGAGAACCTGCTTCTGGC |
| | | CAGCAAGTGCAAAGGGGCTGCAGTGAAGCTGGC |
| | | AGACTTCGGCCTAGCTATCGAGGTGCAGGGGGA |
| | | CCAGCAGGCATGGTTTGGTTTCGCTGGCACACCA |
| | | GGCTACCTGTCCCCTGAGGTCCTTCGCAAAGAG |
| | | GCGTATGGCAAGCCTGTGGACATCTGGGCATGT |
| | | GGGGTGATCCTGTACATCCTGCTCGTGGGCTACC |
| | | CACCCTTCTGGGACGAGGACCAGCACAAGCTGT |
| | | ACCAGCAGATCAAGGCTGGTGCCTATGACTTCC |
| | | CGTCCCCTGAGTGGGACACCGTCACTCCTGAAG |
| | | CCAAAAACCTCATCAACCAGATGCTGACCATCA |
| | | ACCCTGCCAAGCGCATCACAGCCCATGAGGCCC |
| | | TGAAGCACCCGTGGGTCTGCCAACGCTCCACGG |
| | | TAGCATCCATGATGCACAGACAGGAGGATGTGG |
| | | AGTGTCTGAAAAAGTTCAATGCCAGGAGAAAGC |
| | | TCAAGGGAGCCATCCTCACCACCATGCTGGCCA |
| | | CACGGAATTTCTCAGTGGGCAGACAGACCACCG |
| | | CTCCGGCCACAATGTCCACCGCGGCCTCCGGCA |
| | | CCACCATGGGGCTGGTGGAACAAGCCAAGAGTT |
| | | TACTCAACAAGAAAGCAGATGGAGTCAAGCCCC |
| | | AGACGAATAGCACCAAAAACAGTGCAGCCGCCA |
| | | CCAGCCCCAAAGGGACGCTTCCTCCTGCCGCCCT |
| | | GGAGCCTCAAACCACCGTCATCCATAACCCAGT |
| | | GGACGGGATTAAGGAGTCTTCTGACAGTGCCAA |
| | | TACCACCATAGAGGATGAAGACGCTAAAGCCCG |
| | | GAAGCAGGAGATCATTAAGACCACGGAGCAGCT |
| | | CATCGAGGCCGTCAACAACGGTGACTTTGAGGC |
| | | CTACGCGAAAATCTGTGACCCAGGGCTGACCTC |
| | | GTTTGAGCCTGAAGCACTGGGCAACCTGGTTGA |
| | | AGGGATGGACTTCCACAGATTCTACTTCGAGAA |
| | | CCTGCTGGCCAAGAACAGCAAGCCGATCCACAC |
| | | GACCATCCTGAACCCACACGTGCACGTCATTGG |
| | | AGAGGATGCCGCCTGCATCGCTTACATCCGGCTC |
| | | ACGCAGTACATTGACGGGCAGGGCCGGCCCCGC |
| | | ACCAGCCAGTCTGAGGAGACCCGCGTGTGGCAC |
| | | CGCCGCGACGGCAAGTGGCAGAACGTGCACTTC |
| | | CACTGCTCGGGCGCGCCTGTGGCCCCGCTGCAGT |
| | | GA |
| 33 | HUMAN CaMKIIß | MATTVTCTRFTDEYQLYEDIGKGAFSVVRRCVKL |
| | T287D | CTGHEYAAKIINTKKLSARDHQKLEREARICRLLK |
| | amino acid | HSNIVRLHDSISEEGFHYLVFDLVTGGELFEDIVAR |
| | | EYYSEADASHCIQQILEAVLHCHQMGVVHRDLKP |
| | | ENLLLASKCKGAAVKLADFGLAIEVQGDQQAWFG |
| | | FAGTPGYLSPEVLRKEAYGKPVDIWACGVILYILL |
| | | VGYPPFWDEDQHKLYQQIKAGAYDFPSPEWDTVT |
| | | PEAKNLINQMLTINPAKRITAHEALKHPWVCQRST |
| | | VASMMHRQEDVECLKKFNARRKLKGAILTTMLAT |
| | | RNFSVGRQTTAPATMSTAASGTTMGLVEQAKSLL |
| | | NKKADGVKPQTNSTKNSAAATSPKGTLPPAALEP |
| | | QTTVIHNPVDGIKESSDSANTTIEDEDAKARKQEII |
| | | KTTEQLIEAVNNGDFEAYAKICDPGLTSFEPEALGN |
| | | LVEGMDFHRFYFENLLAKNSKPIHTTILNPHVHVIG |
| | | EDAACIAYIRLTQYIDGQGRPRTSQSEETRVWHRR |
| | | DGKWQNVHFHCSGAPVAPLQ |
| 34 | HUMAN CaMKIIß | ATGGCCACCACGGTGACCTGCACCCGCTTCACC |
| | nucleotide | GACGAGTACCAGCTCTACGAGGATATTGGCAAG |
| | | GGGGCTTTCTCTGTGGTCCGACGCTGTGTCAAGC |
| | | TCTGCACCGGCCATGAGTATGCAGCCAAGATCA |
| | | TCAACACCAAGAAGCTGTCAGCCAGAGATCACC |
| | | AGAAGCTGGAGAGAGAGGCTCGGATCTGCCGCC |
| | | TTCTGAAGCATTCCAACATCGTGCGTCTCCACGA |
| | | CAGCATCTCCGAGGAGGGCTTCCACTACCTGGTC |
| | | TTCGATCTGGTCACTGGTGGGGAGCTCTTTGAAG |
| | | ACATTGTGGCGAGAGAGTACTACAGCGAGGCTG |
| | | ATGCCAGTCACTGTATCCAGCAGATCCTGGAGG |
| | | CCGTTCTCCATTGTCACCAAATGGGGGTCGTCCA |
| | | CAGAGACCTCAAGCCGGAGAACCTGCTTCTGGC |
| | | CAGCAAGTGCAAAGGGGCTGCAGTGAAGCTGGC |
| | | AGACTTCGGCCTAGCTATCGAGGTGCAGGGGGA |
| | | CCAGCAGGCATGGTTTGGTTTCGCTGGCACACCA |
| | | GGCTACCTGTCCCCTGAGGTCCTTCGCAAAGAG |
| | | GCGTATGGCAAGCCTGTGGACATCTGGGCATGT |
| | | GGGGTGATCCTGTACATCCTGCTCGTGGGCTACC |
| | | CACCCTTCTGGGACGAGGACCAGCACAAGCTGT |
| | | ACCAGCAGATCAAGGCTGGTGCCTATGACTTCC |
| | | CGTCCCCTGAGTGGGACACCGTCACTCCTGAAG |
| | | CCAAAAACCTCATCAACCAGATGCTGACCATCA |
| | | ACCCTGCCAAGCGCATCACAGCCCATGAGGCCC |
| | | TGAAGCACCCGTGGGTCTGCCAACGCTCCACGG |
| | | TAGCATCCATGATGCACAGACAGGAGACTGTGG |
| | | AGTGTCTGAAAAAGTTCAATGCCAGGAGAAAGC |
| | | TCAAGGGAGCCATCCTCACCACCATGCTGGCCA |
| | | CACGGAATTTCTCAGTGGGCAGACAGACCACCG |
| | | CTCCGGCCACAATGTCCACCGCGGCCTCCGGCA |
| | | CCACCATGGGGCTGGTGGAACAAGCCAAGAGTT |
| | | TACTCAACAAGAAAGCAGATGGAGTCAAGCCCC |
| | | AGACGAATAGCACCAAAAACAGTGCAGCCGCCA |
| | | CCAGCCCCAAAGGGACGCTTCCTCCTGCCGCCCT |
| | | GGAGCCTCAAACCACCGTCATCCATAACCCAGT |
| | | GGACGGGATTAAGGAGTCTTCTGACAGTGCCAA |
| | | TACCACCATAGAGGATGAAGACGCTAAAGCCCG |
| | | GAAGCAGGAGATCATTAAGACCACGGAGCAGCT |
| | | CATCGAGGCCGTCAACAACGGTGACTTTGAGGC |
| | | CTACGCGAAAATCTGTGACCCAGGGCTGACCTC |
| | | GTTTGAGCCTGAAGCACTGGGCAACCTGGTTGA |
| | | AGGGATGGACTTCCACAGATTCTACTTCGAGAA |
| | | CCTGCTGGCCAAGAACAGCAAGCCGATCCACAC |
| | | GACCATCCTGAACCCACACGTGCACGTCATTGG |
| | | AGAGGATGCCGCCTGCATCGCTTACATCCGGCTC |
| | | ACGCAGTACATTGACGGGCAGGGCCGGCCCCGC |
| | | ACCAGCCAGTCTGAGGAGACCCGCGTGTGGCAC |
| | | CGCCGCGACGGCAAGTGGCAGAACGTGCACTTC |
| | | CACTGCTCGGGCGCGCCTGTGGCCCCGCTGCAGT |
| | | GA |
| 35 | HUMAN CaMKIIß | MATTVTCTRFTDEYQLYEDIGKGAFSVVRRCVKL |
| | amino acid | CTGHEYAAKIINTKKLSARDHQKLEREARICRLLK |
| | | HSNIVRLHDSISEEGFHYLVFDLVTGGELFEDIVAR |
| | | EYYSEADASHCIQQILEAVLHCHQMGVVHRDLKP |
| | | ENLLLASKCKGAAVKLADFGLAIEVQGDQQAWFG |
| | | FAGTPGYLSPEVLRKEAYGKPVDIWACGVILYILL |
| | | VGYPPFWDEDQHKLYQQIKAGAYDFPSPEWDTVT |
| | | PEAKNLINQMLTINPAKRITAHEALKHPWVCQRST |
| | | VASMMHRQETVECLKKFNARRKLKGAILTTMLAT |
| | | RNFSVGRQTTAPATMSTAASGTTMGLVEQAKSLL |
| | | NKKADGVKPQTNSTKNSAAATSPKGTLPPAALEP |
| | | QTTVIHNPVDGIKESSDSANTTIEDEDAKARKQEII |
| | | KTTEQLIEAVNNGDFEAYAKICDPGLTSFEPEALGN |
| | | LVEGMDFHRFYFENLLAKNSKPIHTTILNPHVHVIG |
| | | EDAACIAYIRLTQYIDGQGRPRTSQSEETRVWHRR |
| | | DGKWQNVHFHCSGAPVAPLQ |
| 36 | HUMAN CaMKIIα | ATGGCCACCATCACCTGCACCCGCTTCACGGAA |
| | T286D | GAGTACCAGCTCTTCGAGGAATTGGGCAAGGGA |
| | nucleotide | GCCTTCTCGGTGGTGCGAAGGTGTGTGAAGGTG |
| | | CTGGCTGGCCAGGAGTATGCTGCCAAGATCATC |
| | | AACACAAAGAAGCTGTCAGCCAGAGACCATCAG |
| | | AAGCTGGAGCGTGAAGCCCGCATCTGCCGCCTG |
| | | CTGAAGCACCCCAACATCGTCCGACTACATGAC |
| | | AGCATCTCAGAGGAGGGACACCACTACCTGATC |
| | | TTCGACCTGGTCACTGGTGGGGAACTGTTTGAAG |
| | | ATATCGTGGCCCGGGAGTATTACAGTGAGGCGG |
| | | ATGCCAGTCACTGTATCCAGCAGATCCTGGAGG |
| | | CTGTGCTGCACTGCCACCAGATGGGGGTGGTGC |
| | | ACCGGGACCTGAAGCCTGAGAATCTGTTGCTGG |
| | | CCTCCAAGCTCAAGGGTGCCGCAGTGAAGCTGG |
| | | CAGACTTTGGCCTGGCCATAGAGGTGGAGGGGG |
| | | AGCAGCAGGCATGGTTTGGGTTTGCAGGGACTC |
| | | CTGGATATCTCTCCCCAGAAGTGCTGCGGAAGG |
| | | ACCCGTACGGGAAGCCTGTGGACCTGTGGGCTT |
| | | GTGGGGTCATCCTGTACATCCTGCTGGTTGGGTA |
| | | CCCCCCGTTCTGGGATGAGGACCAGCACCGCCT |
| | | GTACCAGCAGATCAAAGCCGGCGCCTATGATTT |
| | | CCCATCGCCGGAATGGGACACTGTCACCCCGGA |
| | | AGCCAAGGATCTGATCAATAAGATGCTGACCAT |
| | | TAACCCATCCAAACGCATCACAGCTGCCGAAGC |
| | | CCTTAAGCACCCCTGGATCTCGCACCGCTCCACC |
| | | GTGGCATCCTGCATGCACAGACAGGAGGACGTG |
| | | GACTGCCTGAAGAAGTTCAATGCCAGGAGGAAA |
| | | CTGAAGGGAGCCATTCTCACCACGATGCTGGCC |
| | | ACCAGGAACTTCTCCGGAGGGAAGAGTGGGGGA |
| | | AACAAGAAGAGCGATGGTGTGAAGGAATCCTCA |
| | | GAGAGCACCAACACCACCATCGAGGATGAAGAC |
| | | ACCAAAGTGCGGAAACAGGAAATTATAAAAGTG |
| | | ACAGAGCAGCTGATTGAAGCCATAAGCAATGGA |
| | | GATTTTGAGTCCTACACGAAGATGTGCGACCCTG |
| | | GCATGACAGCCTTCGAACCTGAGGCCCTGGGGA |
| | | ACCTGGTTGAGGGCCTGGACTTCCATCGATTCTA |
| | | TTTTGAAAACCTGTGGTCCCGGAACAGCAAGCC |
| | | CGTGCACACCACCATCCTGAATCCCCACATCCAC |
| | | CTGATGGGCGACGAGTCAGCCTGCATCGCCTAC |
| | | ATCCGCATCACGCAGTACCTGGACGCTGGCGGC |
| | | ATCCCACGCACCGCCCAGTCGGAGGAGACCCGT |
| | | GTCTGGCACCGCCGGGATGGCAAATGGCAGATC |
| | | GTCCACTTCCACAGATCTGGGGCGCCCTCCGTCC |
| | | TGCCCCACTGA |
| 37 | HUMAN CaMKIIα | MATITCTRFTEEYQLFEELGKGAFSVVRRCVKVLA |
| | T286D | GQEYAAKIINTKKLSARDHQKLEREARICRLLKHP |
| | amino acid | NIVRLHDSISEEGHHYLIFDLVTGGELFEDIVAREY |
| | | YSEADASHCIQQILEAVLHCHQMGVVHRDLKPEN |
| | | LLLASKLKGAAVKLADFGLAIEVEGEQQAWFGFA |
| | | GTPGYLSPEVLRKDPYGKPVDLWACGVILYILLVG |
| | | YPPFWDEDQHRLYQQIKAGAYDFPSPEWDTVTPE |
| | | AKDLINKMLTINPSKRITAAEALKHPWISHRSTVAS |
| | | CMHRQEDVDCLKKFNARRKLKGAILTTMLATRNF |
| | | SGGKSGGNKKSDGVKESSESTNTTIEDEDTKVRKQ |
| | | EIIKVTEQLIEAISNGDFESYTKMCDPGMTAFEPEA |
| | | LGNLVEGLDFHRFYFENLWSRNSKPVHTTILNPHI |
| | | HLMGDESACIAYIRITQYLDAGGIPRTAQSEETRV |
| | | WHRRDGKWQIVHFHRSGAPSVLPH |
| 38 | HUMAN CaMKIIα | ATGGCCACCATCACCTGCACCCGCTTCACGGAA |
| | nucleotide | GAGTACCAGCTCTTCGAGGAATTGGGCAAGGGA |
| | | GCCTTCTCGGTGGTGCGAAGGTGTGTGAAGGTG |
| | | CTGGCTGGCCAGGAGTATGCTGCCAAGATCATC |
| | | AACACAAAGAAGCTGTCAGCCAGAGACCATCAG |
| | | AAGCTGGAGCGTGAAGCCCGCATCTGCCGCCTG |
| | | CTGAAGCACCCCAACATCGTCCGACTACATGAC |
| | | AGCATCTCAGAGGAGGGACACCACTACCTGATC |
| | | TTCGACCTGGTCACTGGTGGGGAACTGTTTGAAG |
| | | ATATCGTGGCCCGGGAGTATTACAGTGAGGCGG |
| | | ATGCCAGTCACTGTATCCAGCAGATCCTGGAGG |
| | | CTGTGCTGCACTGCCACCAGATGGGGGTGGTGC |
| | | ACCGGGACCTGAAGCCTGAGAATCTGTTGCTGG |
| | | CCTCCAAGCTCAAGGGTGCCGCAGTGAAGCTGG |
| | | CAGACTTTGGCCTGGCCATAGAGGTGGAGGGGG |
| | | AGCAGCAGGCATGGTTTGGGTTTGCAGGGACTC |
| | | CTGGATATCTCTCCCCAGAAGTGCTGCGGAAGG |
| | | ACCCGTACGGGAAGCCTGTGGACCTGTGGGCTT |
| | | GTGGGGTCATCCTGTACATCCTGCTGGTTGGGTA |
| | | CCCCCCGTTCTGGGATGAGGACCAGCACCGCCT |
| | | GTACCAGCAGATCAAAGCCGGCGCCTATGATTT |
| | | CCCATCGCCGGAATGGGACACTGTCACCCCGGA |
| | | AGCCAAGGATCTGATCAATAAGATGCTGACCAT |
| | | TAACCCATCCAAACGCATCACAGCTGCCGAAGC |
| | | CCTTAAGCACCCCTGGATCTCGCACCGCTCCACC |
| | | GTGGCATCCTGCATGCACAGACAGGAGACCGTG |
| | | GACTGCCTGAAGAAGTTCAATGCCAGGAGGAAA |
| | | CTGAAGGGAGCCATTCTCACCACGATGCTGGCC |
| | | ACCAGGAACTTCTCCGGAGGGAAGAGTGGGGGA |
| | | AACAAGAAGAGCGATGGTGTGAAGGAATCCTCA |
| | | GAGAGCACCAACACCACCATCGAGGATGAAGAC |
| | | ACCAAAGTGCGGAAACAGGAAATTATAAAAGTG |
| | | ACAGAGCAGCTGATTGAAGCCATAAGCAATGGA |
| | | GATTTTGAGTCCTACACGAAGATGTGCGACCCTG |
| | | GCATGACAGCCTTCGAACCTGAGGCCCTGGGGA |
| | | ACCTGGTTGAGGGCCTGGACTTCCATCGATTCTA |
| | | TTTTGAAAACCTGTGGTCCCGGAACAGCAAGCC |
| | | CGTGCACACCACCATCCTGAATCCCCACATCCAC |
| | | CTGATGGGCGACGAGTCAGCCTGCATCGCCTAC |
| | | ATCCGCATCACGCAGTACCTGGACGCTGGCGGC |
| | | ATCCCACGCACCGCCCAGTCGGAGGAGACCCGT |
| | | GTCTGGCACCGCCGGGATGGCAAATGGCAGATC |
| | | GTCCACTTCCACAGATCTGGGGCGCCCTCCGTCC |
| | | TGCCCCACTGA |
| 39 | HUMAN CaMKIIα | MATITCTRFTEEYQLFEELGKGAFSVVRRCVKVLA |
| | amino acid | GQEYAAKIINTKKLSARDHQKLEREARICRLLKHP |
| | | NIVRLHDSISEEGHHYLIFDLVTGGELFEDIVAREY |
| | | YSEADASHCIQQILEAVLHCHQMGVVHRDLKPEN |
| | | LLLASKLKGAAVKLADFGLAIEVEGEQQAWFGFA |
| | | GTPGYLSPEVLRKDPYGKPVDLWACGVILYILLVG |
| | | YPPFWDEDQHRLYQQIKAGAYDFPSPEWDTVTPE |
| | | AKDLINKMLTINPSKRITAAEALKHPWISHRSTVAS |
| | | CMHRQETVDCLKKFNARRKLKGAILTTMLATRNF |
| | | SGGKSGGNKKSDGVKESSESTNTTIEDEDTKVRKQ |
| | | EIIKVTEQLIEAISNGDFESYTKMCDPGMTAFEPEA |
| | | LGNLVEGLDFHRFYFENLWSRNSKPVHTTILNPHI |
| | | HLMGDESACIAYIRITQYLDAGGIPRTAQSEETRV |
| | | WHRRDGKWQIVHFHRSGAPSVLPH |
| 40 | HUMAN CaMKIIß | ATGGCCACCACGGTGACCTGCACCCGCTTCACC |
| | N-terminal catalytic | GACGAGTACCAGCTCTACGAGGATATTGGCAAG |
| | domain | GGGGCTTTCTCTGTGGTCCGACGCTGTGTCAAGC |
| | nucleotide | TCTGCACCGGCCATGAGTATGCAGCCAAGATCA |
| | | TCAACACCAAGAAGCTGTCAGCCAGAGATCACC |
| | | AGAAGCTGGAGAGAGAGGCTCGGATCTGCCGCC |
| | | TTCTGAAGCATTCCAACATCGTGCGTCTCCACGA |
| | | CAGCATCTCCGAGGAGGGCTTCCACTACCTGGTC |
| | | TTCGATCTGGTCACTGGTGGGGAGCTCTTTGAAG |
| | | ACATTGTGGCGAGAGAGTACTACAGCGAGGCTG |
| | | ATGCCAGTCACTGTATCCAGCAGATCCTGGAGG |
| | | CCGTTCTCCATTGTCACCAAATGGGGGTCGTCCA |
| | | CAGAGACCTCAAGCCGGAGAACCTGCTTCTGGC |
| | | CAGCAAGTGCAAAGGGGCTGCAGTGAAGCTGGC |
| | | AGACTTCGGCCTAGCTATCGAGGTGCAGGGGGA |
| | | CCAGCAGGCATGGTTTGGTTTCGCTGGCACACCA |
| | | GGCTACCTGTCCCCTGAGGTCCTTCGCAAAGAG |
| | | GCGTATGGCAAGCCTGTGGACATCTGGGCATGT |
| | | GGGGTGATCCTGTACATCCTGCTCGTGGGCTACC |
| | | CACCCTTCTGGGACGAGGACCAGCACAAGCTGT |
| | | ACCAGCAGATCAAGGCTGGTGCCTATGACTTCC |
| | | CGTCCCCTGAGTGGGACACCGTCACTCCTGAAG |
| | | CCAAAAACCTCATCAACCAGATGCTGACCATCA |
| | | ACCCTGCCAAGCGCATCACAGCCCATGAGGCCC |
| | | TGAAGCACCCGTGGGTCTGCCAACGCTCCACGG |
| | | TAGCATCCATGATGCACAGACAGGAGACTGTGG |
| | | AGTGTCTGTGA |
| 41 | HUMAN CaMKIIß | MATTVTCTRFTDEYQLYEDIGKGAFSVVRRCVKL |
| | N-terminal catalytic | CTGHEYAAKIINTKKLSARDHQKLEREARICRLLK |
| | domain | HSNIVRLHDSISEEGFHYLVFDLVTGGELFEDIVAR |
| | amino acid | EYYSEADASHCIQQILEAVLHCHQMGVVHRDLKP |
| | | ENLLLASKCKGAAVKLADFGLAIEVQGDQQAWFG |
| | | FAGTPGYLSPEVLRKEAYGKPVDIWACGVILYILL |
| | | VGYPPFWDEDQHKLYQQIKAGAYDFPSPEWDTVT |
| | | PEAKNLINQMLTINPAKRITAHEALKHPWVCQRST |
| | | VASMMHRQETVECL |
| 42 | HUMAN CaMKIIα | ATGGCCACCATCACCTGCACCCGCTTCACGGAA |
| | N-terminal catalytic | GAGTACCAGCTCTTCGAGGAATTGGGCAAGGGA |
| | domain | GCCTTCTCGGTGGTGCGAAGGTGTGTGAAGGTG |
| | nucleotide | CTGGCTGGCCAGGAGTATGCTGCCAAGATCATC |
| | | AACACAAAGAAGCTGTCAGCCAGAGACCATCAG |
| | | AAGCTGGAGCGTGAAGCCCGCATCTGCCGCCTG |
| | | CTGAAGCACCCCAACATCGTCCGACTACATGAC |
| | | AGCATCTCAGAGGAGGGACACCACTACCTGATC |
| | | TTCGACCTGGTCACTGGTGGGGAACTGTTTGAAG |
| | | ATATCGTGGCCCGGGAGTATTACAGTGAGGCGG |
| | | ATGCCAGTCACTGTATCCAGCAGATCCTGGAGG |
| | | CTGTGCTGCACTGCCACCAGATGGGGGTGGTGC |
| | | ACCGGGACCTGAAGCCTGAGAATCTGTTGCTGG |
| | | CCTCCAAGCTCAAGGGTGCCGCAGTGAAGCTGG |
| | | CAGACTTTGGCCTGGCCATAGAGGTGGAGGGGG |
| | | AGCAGCAGGCATGGTTTGGGTTTGCAGGGACTC |
| | | CTGGATATCTCTCCCCAGAAGTGCTGCGGAAGG |
| | | ACCCGTACGGGAAGCCTGTGGACCTGTGGGCTT |
| | | GTGGGGTCATCCTGTACATCCTGCTGGTTGGGTA |
| | | CCCCCCGTTCTGGGATGAGGACCAGCACCGCCT |
| | | GTACCAGCAGATCAAAGCCGGCGCCTATGATTT |
| | | CCCATCGCCGGAATGGGACACTGTCACCCCGGA |
| | | AGCCAAGGATCTGATCAATAAGATGCTGACCAT |
| | | TAACCCATCCAAACGCATCACAGCTGCCGAAGC |
| | | CCTTAAGCACCCCTGGATCTCGCACCGCTCCACC |
| | | GTGGCATCCTGCATGCACAGACAGGAGACCGTG |
| | | GACTGCCTGTGA |
| 43 | HUMAN CaMKIIα | MATITCTRFTEEYQLFEELGKGAFSVVRRCVKVLA |
| | N-terminal catalytic | GQEYAAKIINTKKLSARDHQKLEREARICRLLKHP |
| | domain | NIVRLHDSISEEGHHYLIFDLVTGGELFEDIVAREY |
| | amino acid | YSEADASHCIQQILEAVLHCHQMGVVHRDLKPEN |
| | | LLLASKLKGAAVKLADFGLAIEVEGEQQAWFGFA |
| | | GTPGYLSPEVLRKDPYGKPVDLWACGVILYILLVG |
| | | YPPFWDEDQHRLYQQIKAGAYDFPSPEWDTVTPE |
| | | AKDLINKMLTINPSKRITAAEALKHPWISHRSTVAS |
| | | CMHRQETVDCL |
| 44 | HUMAN CaMKI | ATGCTGGGGGCAGTGGAAGGCCCCAGGTGGAAG |
| | N-terminal catalytic | CAGGCGGAGGACATTAGAGACATCTACGACTTC |
| | domain | CGAGATGTTCTGGGCACGGGGGCCTTCTCGGAG |
| | nucleotide | GTGATCCTGGCAGAAGATAAGAGGACGCAGAAG |
| | | CTGGTGGCCATCAAATGCATTGCCAAGGAGGCC |
| | | CTGGAGGGCAAGGAAGGCAGCATGGAGAATGA |
| | | GATTGCTGTCCTGCACAAGATCAAGCACCCCAA |
| | | CATTGTAGCCCTGGATGACATCTATGAGAGTGG |
| | | GGGCCACCTCTACCTCATCATGCAGCTGGTGTCG |
| | | GGTGGGGAGCTCTTTGACCGTATTGTGGAAAAA |
| | | GGCTTCTACACGGAGCGGGACGCCAGCCGCCTC |
| | | ATCTTCCAGGTGCTGGATGCTGTGAAATACCTGC |
| | | ATGACCTGGGCATTGTACACCGGGATCTCAAGC |
| | | CAGAGAATCTGCTGTACTACAGCCTGGATGAAG |
| | | ACTCCAAAATCATGATCTCCGACTTTGGCCTCTC |
| | | CAAGATGGAGGACCCGGGCAGTGTGCTCTCCAC |
| | | CGCCTGTGGAACTCCGGGATACGTGGCCCCTGA |
| | | AGTCCTGGCCCAGAAGCCCTACAGCAAGGCTGT |
| | | GGATTGCTGGTCCATAGGTGTCATCGCCTACATC |
| | | TTGCTCTGCGGTTACCCTCCCTTCTATGACGAGA |
| | | ATGATGCCAAACTCTTTGAACAGATTTTGAAGGC |
| | | CGAGTACGAGTTTGACTCTCCTTACTGGGACGAC |
| | | ATCTCTGACTCTGCCAAAGATTTCATCCGGCACT |
| | | TGATGGAGAAGGACCCAGAGAAAAGATTCACCT |
| | | GTGAGCAGGCCTTGCAGCACCCATGGATTGCAG |
| | | GAGATACAGCTCTAGATAAGAATATCCACCAGT |
| | | CGGTGAGTGAGCAGTAG |
| 45 | HUMAN CaMKI | MLGAVEGPRWKQAEDIRDIYDFRDVLGTGAFSEVI |
| | N-terminal catalytic | LAEDKRTQKLVAIKCIAKEALEGKEGSMENEIAVL |
| | domain | HKIKHPNIVALDDIYESGGHLYLIMQLVSGGELFDR |
| | amino acid | IVEKGFYTERDASRLIFQVLDAVKYLHDLGIVHRD |
| | | LKPENLLYYSLDEDSKIMISDFGLSKMEDPGSVLST |
| | | ACGTPGYVAPEVLAQKPYSKAVDCWSIGVIAYILL |
| | | CGYPPFYDENDAKLFEQILKAEYEFDSPYWDDISD |
| | | SAKDFIRHLMEKDPEKRFTCEQALQHPWIAGDTAL |
| | | DKNIHQSVSEQ |
| 46 | HUMAN CaMKI | ATGCTGGGGGCAGTGGAAGGCCCCAGGTGGAAG |
| | nucleotide | CAGGCGGAGGACATTAGAGACATCTACGACTTC |
| | | CGAGATGTTCTGGGCACGGGGGCCTTCTCGGAG |
| | | GTGATCCTGGCAGAAGATAAGAGGACGCAGAAG |
| | | CTGGTGGCCATCAAATGCATTGCCAAGGAGGCC |
| | | CTGGAGGGCAAGGAAGGCAGCATGGAGAATGA |
| | | GATTGCTGTCCTGCACAAGATCAAGCACCCCAA |
| | | CATTGTAGCCCTGGATGACATCTATGAGAGTGG |
| | | GGGCCACCTCTACCTCATCATGCAGCTGGTGTCG |
| | | GGTGGGGAGCTCTTTGACCGTATTGTGGAAAAA |
| | | GGCTTCTACACGGAGCGGGACGCCAGCCGCCTC |
| | | ATCTTCCAGGTGCTGGATGCTGTGAAATACCTGC |
| | | ATGACCTGGGCATTGTACACCGGGATCTCAAGC |
| | | CAGAGAATCTGCTGTACTACAGCCTGGATGAAG |
| | | ACTCCAAAATCATGATCTCCGACTTTGGCCTCTC |
| | | CAAGATGGAGGACCCGGGCAGTGTGCTCTCCAC |
| | | CGCCTGTGGAACTCCGGGATACGTGGCCCCTGA |
| | | AGTCCTGGCCCAGAAGCCCTACAGCAAGGCTGT |
| | | GGATTGCTGGTCCATAGGTGTCATCGCCTACATC |
| | | TTGCTCTGCGGTTACCCTCCCTTCTATGACGAGA |
| | | ATGATGCCAAACTCTTTGAACAGATTTTGAAGGC |
| | | CGAGTACGAGTTTGACTCTCCTTACTGGGACGAC |
| | | ATCTCTGACTCTGCCAAAGATTTCATCCGGCACT |
| | | TGATGGAGAAGGACCCAGAGAAAAGATTCACCT |
| | | GTGAGCAGGCCTTGCAGCACCCATGGATTGCAG |
| | | GAGATACAGCTCTAGATAAGAATATCCACCAGT |
| | | CGGTGAGTGAGCAGATCAAGAAGAACTTTGCCA |
| | | AGAGCAAGTGGAAGCAAGCCTTCAATGCCACGG |
| | | CTGTGGTGCGGCACATGAGGAAACTGCAGCTGG |
| | | GCACCAGCCAGGAGGGGCAGGGGCAGACGGCG |
| | | AGCCATGGGGAGCTGCTGACACCAGTGGCTGGG |
| | | GGGCCGGCAGCTGGCTGTTGCTGTCGAGACTGC |
| | | TGCGTGGAGCCGGGCACAGAACTGTCCCCCACA |
| | | CTGCCCCACCAGCTCTAG |
| 47 | HUMAN CaMKI | MLGAVEGPRWKQAEDIRDIYDFRDVLGTGAFSEVI |
| | amino acid | LAEDKRTQKLVAIKCIAKEALEGKEGSMENEIAVL |
| | | HKIKHPNIVALDDIYESGGHLYLIMQLVSGGELFDR |
| | | IVEKGFYTERDASRLIFQVLDAVKYLHDLGIVHRD |
| | | LKPENLLYYSLDEDSKIMISDFGLSKMEDPGSVLST |
| | | ACGTPGYVAPEVLAQKPYSKAVDCWSIGVIAYILL |
| | | CGYPPFYDENDAKLFEQILKAEYEFDSPYWDDISD |
| | | SAKDFIRHLMEKDPEKRFTCEQALQHPWIAGDTAL |
| | | DKNIHQSVSEQIKKNFAKSKWKQAFNATAVVRHM |
| | | RKLQLGTSQEGQGQTASHGELLTPVAGGPAAGCC |
| | | CRDCCVEPGTELSPTLPHQL |

In an example, a CaMK or polynucleotide encoding a CaMK may have less than 100% sequence homology with a CaMK or polynucleotide encoding a CaMK disclosed in Table 2. In an example, a CaMK or polynucleotide encoding a CaMK may have at least about 60% sequence homology with a CaMK or polynucleotide encoding a CaMK disclosed in Table 2, at least about 65% sequence homology with a CaMK or polynucleotide encoding a CaMK disclosed in Table 2, at least about 70% sequence homology with a CaMK or polynucleotide encoding a CaMK disclosed in Table 2, at least about 75% sequence homology with a CaMK or polynucleotide encoding a CaMK disclosed in Table 2, at least about 80% sequence homology with a CaMK or polynucleotide encoding a CaMK disclosed in Table 2, at least about 85% sequence homology with a CaMK or polynucleotide encoding a CaMK disclosed in Table 2, at least about 90% sequence homology with a CaMK or polynucleotide encoding a CaMK disclosed in Table 2, at least about 95% sequence homology with a CaMK or polynucleotide encoding a CaMK disclosed in Table 2, at least about 97% sequence homology with a CaMK or polynucleotide encoding a CaMK disclosed in Table 2, or at least about 99% sequence homology with a CaMK or polynucleotide encoding a CaMK disclosed in Table 2.

In an example, a CREB or polynucleotide encoding a CREB may have less than 100% sequence homology with a CREB or polynucleotide encoding a CREB disclosed in Table 2. In an example, a CREB or polynucleotide encoding a CREB may have at least about 60% sequence homology with a CREB or polynucleotide encoding a CREB disclosed in Table 2, at least about 65% sequence homology with a CREB or polynucleotide encoding a CREB disclosed in Table 2, at least about 70% sequence homology with a CREB or polynucleotide encoding a CREB disclosed in Table 2, at least about 75% sequence homology with a CREB or polynucleotide encoding a CREB disclosed in Table 2, at least about 80% sequence homology with a CREB or polynucleotide encoding a CREB disclosed in Table 2, at least about 85% sequence homology with a CREB or polynucleotide encoding a CREB disclosed in Table 2, at least about 90% sequence homology with a CREB or polynucleotide encoding a CREB disclosed in Table 2, at least about 95% sequence homology with a CREB or polynucleotide encoding a CREB disclosed in Table 2, at least about 97% sequence homology with a CREB or polynucleotide encoding a CREB disclosed in Table 2, or at least about 99% sequence homology with a CREB or polynucleotide encoding a CREB disclosed in Table 2.

Explicitly disclosed herein, without limitation, are examples including any of the promoters disclosed in Table 1, or any other RGC promoter, driving expression of any of the CaMK or CREB disclosed in Table. Also explicitly included herein, without limitation, is use of a compound in the manufacture of a medicament for use of prevention of RGCs, or in treating vision loss, in any subject as disclosed herein, wherein the compound comprises a CaMK or CREB protein or variations thereof, or nucleotide sequence or variation thereof, and optionally any RGC promoter or other promoter disclosed herein, and may include a vector including any combination of any of the foregoing, as disclosed herein for use in preventing degeneration of RGC or treatment of vision loss herein.

### NON-LIMITING WORKING EXAMPLES

The following examples are intended to illustrate particular embodiments of the present disclosure, but are by no means intended to limit the scope thereof.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

### Materials and Methods

### Animals

C57BL/6 mice were purchased from The Jackson Laboratory (Bar Harbor, Maine). GLAST−/− mice were provided by Dr. Kohichi Tanaka, Tokyo Medical & Dental University (TMDU). All studies adhered to the procedures consistent with animal protocols approved by the IACUC at the Icahn School of Medicine at Mount Sinai. Male mice were used for the magnetic microbeads occlusion model; mice of either sex were randomly assigned to different groups for other experiments.

### AAV Plasmids Construction and AAV Preparation

pAAV-GFP plasmids were kindly provided by Dr. Kevin Park (University of Miami). For AAV plasmid construction, the protein-coding region of pAAV-GFP was replaced by cDNAs of the following plasmids: CaMKIIα WT (Addgene #21226), CaMKIIα K42R (Addgene #21221), CaMKIIα T286D (Addgene #16736), CaMKIIβ WT (Addgene #21227), CaMKIIβ K43R (Addgene #21225), CaMKIIβ T287D (Addgene #21223) and VP16-CREB and A-CREB (both from Dr. Hongbing Wang, Michigan State University). GenScript (Piscataway, NJ) generated CaMKIIα K42D and CaMKIIα T286A from CaMKIIα WT, CaMKIIα T286D/T305A/T306A and CaMKIIα T286D/T305D/T306D from CaMKIIα T286D, CaMKIIβ K43D and CaMKIIβ T287A from CaMKIIβ WT, CaMKIIβ T287D/T306A/T307A and CaMKIIβ T287D/T306D/T307D from CaMKIIβ T287D. AAV-mSncg-GFP was kindly provided by Dr. Yang Hu (Stanford University) and used to generate AAV-mSncg-EBFP and AAV-mSncg-CaMKIIα T286D. AAV Rap-Cap and Helper plasmids were used for co-transfection in AAVpro 293T Cell Line (Takara Bio, 632273). Discontinuous iodixanol gradient ultracentrifugation was used to purify AAV. AAV titers, determined by real-time PCR, were in the range of 1-4×10¹³ genome copies per milliliter.

### Intravitreal Injection and Optic Nerve Crush

Adult mice were anesthetized with a mixture of ketamine (100 mg/kg) and xylazine (10 mg/kg) by intraperitoneal injection. GLAST−/− mouse pups were anesthetized by chilling on ice, and a small incision was made in the eyelid with a 30-gauge needle to expose the eyeball. For intravitreal injection, the micropipette was inserted just behind the ora serrata, and AAV or other solution was injected into the vitreous body. N-Methyl-D-aspartic Acid (Millipore Sigma, 454575) and Myristoylated Autocamtide-2-Related Inhibitory Peptide (Millipore Sigma, 189482) was prepared in PBS. For optic nerve crush, the optic nerve was exposed and crushed intraorbitally with jeweler's forceps for 5 s approximately 1 mm behind the globe. Ophthalmic ointment was applied to protect the cornea after surgery.

### Induction of IOP Elevation in Mice

To elevate IOP, 3-month-old mice were anesthetized by a mixture of ketamine and xylazine. Proparacaine Hydrochloride eye drops were used before surgery. Magnetic microbeads (Dynabeads® M-450 Epoxy, Thermo Fisher Scientific) were injected unilaterally into the anterior chamber and distributed evenly around the circumference of the anterior chamber using magnets, as described recently (Ito et al., 2016). The IOP of both eyes was monitored using the TonoLab tonometer according to manufacturer's instructions.

### Histology and Microscopy

For immunohistochemistry, eyes with the attached optic nerve segment, surgically removed from perfused mice, were post-fixed in 4% PFA. Retinas were dissected out for whole-mount staining. Retinal whole-mounts were blocked in the staining buffer containing 5% normal donkey serum and 0.1% Triton X-100 in PBS for 1 hr. Retinas were incubated with primary antibodies overnight and washed 3×15 minutes with PBS before 2 hours of incubation with secondary antibodies at room temperature. Retinas were again washed 3×15 minutes with PBS and then mounted with Fluoromount-G. Primary antibodies used: Tuj1 (1:250, Biolegend, 801202), pCaMKII (1:100, Abcam, ab32678), pan-CaMKII (1:100, Abcam, ab52476), pCREB (1:100, Abcam, ab32096), pTrkB (1:100, Thermo Fisher Scientific, MA5-32207), DLK (1:100, Thermo Fisher Scientific, PA5-32173), p-c-Jun (1:100, Cell Signaling Technology, 2361). The peptide used: CaMKII alpha (phospho T286) peptide (12.5 μg/ml, Abcam, ab 115237). Secondary antibodies used: Alexa Fluor® 488 AffiniPure Donkey Anti-Mouse IgG (1:500, Jackson ImmunoResearch Labs, 715-545-151), Alexa Fluor® 594 AffiniPure Donkey Anti-Mouse IgG (1:500, Jackson ImmunoResearch Labs, 715-585-151), Cy™3 AffiniPure Donkey Anti-Rabbit IgG (1:500, Jackson ImmunoResearch Labs, 711-165-152), Alexa Fluor® 594 AffiniPure Donkey Anti-Rabbit IgG (1:500, Jackson ImmunoResearch Labs, 711-585-152), Alexa Fluor® 647 AffiniPure Donkey Anti-Rabbit IgG (1:500, Jackson ImmunoResearch Labs, 711-605-152). Confocal images were acquired using a Zeiss LSM 800 microscope. For RGC counting and signaling pathway studies, squares (320×320 μm) were sampled around the peripheral region of each retina whole-mount (˜500 μm from square center to retina edge) for analysis.

For visual pathway CTB tracing, 1.5 μl of Cholera Toxin Subunit B (CTB, Alexa Fluor™ 488 Conjugate, Thermo Fisher Scientific, C22841) (2 μg/μl in PBS) was injected into the vitreous. Three days after CTB injection, animals were perfused with 4% PFA. Optic nerves were dissected, fixed, and mounted for imaging. Brains were dissected, fixed and mounted in 3% agarose. Brain slices (150 μm thickness) were sectioned coronally for LGN or sagittally for superior colliculus using a vibratome (1000VT, Leica) and mounted for imaging as previously reported (Zhang et al., 2011). Confocal images were acquired using a Zeiss LSM 800 microscope.

For visualization of microbeads accumulation at the iridocorneal angle, eyes from perfused mice were collected and post-fixed in 4% PFA. The anterior part of each eye was dissected out, embedded in OCT compound, sectioned using a cryostat, stained with H&E, and imaged with Zeiss LSM 800 microscope equipped with a color camera.

For axon survival examination of microbeads-injected eyes, optic nerves were fixed in 2.5% glutaraldehyde and 2% paraformaldehyde in 0. IM sodium cacodylate buffer for 0.5 hours at room temperature and then 2 hours at 4 deg C. Optic nerve regions 1 mm distal to the eyeball were embedded in resin. Semithin sections of the optic nerve were stained with toluidine blue and imaged with Zeiss LSM 800 microscope equipped with a color camera through a 100× lens. Square areas (22×22 μm) were sampled around the peripheral region of each nerve section (˜50 μm from square center to nerve edge) for analysis.

For optic nerve head analysis, eyes with the attached optic nerve segment, surgically removed from perfused mice, were post-fixed in 4% PFA. After removal of cornea, iris, and lens, eyes were embedded in OCT compound for cryosection. Sections through the optic nerve were collected, stained with DAPI (Thermo Fisher Scientific, 62248), and imaged with Zeiss LSM 800 microscope.

Images were analyzed and organized using ImageJ (Schindelin et al., 2012) and Photoshop.

### RGC Purification and Immunoblotting

For RGC purification, dissected retinas were digested in papain, and dissociated to single cells by gentle pipetting. Retinal cell suspensions were washed in HBSS once, resuspended in HBSS+4% BSA and incubated with PE-Cyanine7 conjugated CD90.2 (Thy-1.2) Antibody (1:2,000, Thermo Fisher Scientific, 25-0902-81) for 15 min to label RGCs for cell sorting (Lu et al., 2020). After another wash with excess HBSS and resuspension in HBSS+4% BSA, DAPI (1 mg/ml, 1:1000, Thermo Fisher Scientific, 62248) was added before filtering to label dead cells. Fluorescence activated cell sorting (FACS) were performed on BD FACSAria II sorter (BD Biosciences) to collect RGCs.

For immunoblotting, purified RGCs were lysed by heated at 95° C. in Laemmli Sample Buffer. Proteins were separated by SDS-PAGE and electro-transferred onto PVDF membranes. Antibodies: pCaMKII (1:1000, Abcam, ab32678), WesternSure Goat anti-Rabbit HRP (1:50000, LI-COR Biosciences, 926-80011), Recombinant HRP Anti-GAPDH antibody (1:400000, Abcam, ab201822). SuperSignal™ West Atto Ultimate Sensitivity Substrate (Thermo Fisher Scientific, A38555) and ChemiDoc Touch Imaging System (Bio-Rad) were used for chemiluminescence detection. Images were analyzed using ImageJ (Schindelin et al., 2012) and Photoshop.

### Pattern Electroretinography (PERG)

PERG was recorded as previously reported using the JORVEC PERG system (Chou et al., 2014; Williams et al., 2017). Mice were anesthetized using a mixture of ketamine/xylazine. The body temperature of the animal was maintained at 37° C. with a feedback-controlled heating stage and monitored using a rectal probe. A small drop of balanced saline was applied topically as necessary to prevent corneal desiccation. The PERG signals were recorded from a subcutaneous stainless steel needle placed in the snout in response to contrast-reversal of gratings (0.05 cycles/degree, 100% contrast) generated on two LED tablets alternating at slightly different frequencies. The reference and ground electrodes were similar needles placed medially on the back of the head and at the root of the tail, respectively. Electrical signals were amplified 10,000 times and band-pass filtered (1-300 Hz). Independent PERG responses were retrieved using an asynchronous averaging method. AAV injection and NMDA treatment was performed unilaterally on one eye for each mouse.

### Pattern Visually Evoked Potentials (PVEP)

For PVEP recordings, stainless-steel microscrews (0.8 mm OD, 120 threads per inch, NAS721CE80-120, Antrin Miniature Specialties) were implanted in the mouse skull at 2 mm rostral to bregma for reference electrode) and 2 mm horizontal to lambda (overlying the contralateral primary visual cortex, for the active electrodes) 3 days after intravitreal NMDA injection. Microscrew advancement was set to 400 μm cortical depth, since at this depth PVEP has the maximal amplitude (Porciatti et al., 1999). PVEPs were recorded one week after the embedding surgery. Mice were anesthetized and kept warm during the recording process. A small drop of balanced saline was applied topically as necessary to prevent corneal desiccation. Electrodes with an alligator clip (ETL-36RSAF, The Electrode Store) were used to connect screws in the skull. The ground electrode was placed at the root of the tail. The JORVEC system was used to display patterned stimuli (gratings of 0.05 cycles/degree, 100% contrast) and collect PVEP signals. Electrical signals were amplified 10,000 times and band-pass filtered (1-100 Hz). Typical PVEP response displays a major negative wave peaking at about 100 ms (Porciatti et al., 1999). AAV injection and NMDA treatment was performed unilaterally on one eye for each mouse. The contralateral eye was light-blocked, and its display screen was turned off to eliminate any possible contribution.

### Acuity Measurement in the Visual Water Task

Visual acuity was measured using the Acumen system (Cerebral Mechanics) as previously reported (Ecker et al., 2010; Prusky et al., 2000). The task was performed in a trapezoidal-shaped tank containing shallow water. There are two display screens on either side of the wide end of the tank. A stationary, vertically oriented sinusoidal grating was displayed randomly on one of two screens, while a homogeneous gray image of the same mean luminance was displayed on the other screen. Animals are trained to swim from the narrow end toward the wide end screens, and at a fixed distance, choose the screen displaying the grating and escape to a submerged platform hidden below it. The training grating was set at the spatial frequency of 0.054 cycles/degree. During testing, the spatial frequency of the grating was increased slowly at the interval of 0.018 cycles/degree until a break, where the animal made fewer than 7 correct choices in 10 trials. At least 4 breaks close together are required to determine that the animal has reached its visual acuity (spatial frequency threshold). The cumulative percentage of correct choices at each spatial frequency was calculated for a scatter plot. Trendline of best fit was generated and the point on the curve that intersected with 70% correct was adopted as the acuity threshold. Mice were trained and their visual acuity was measured before NMDA injection to induce damage. From day 1 to day 3 after NMDA injection, mice were tested daily at low (training) spatial frequency to maintain their training activity. From day 4 to day 14 after NMDA injection, we changed the spatial frequency until the threshold (acuity) was determined for each mouse.

### Visual Cliff Test

The visual cliff apparatus was purchased from Conduct Science (Boston, MA). The Visual cliff test apparatus consists of a clear plexiglass box, in a dimension of 62×62×19 cm, separated by a center platform (1.5 inches high and 2 inches wide) into two regions, the shallow side with a checkered pattern immediately under it, and the deep side with a same checkered pattern placed 2 feet under it to create the illusion of depth (Fox, 1965; Gu et al., 2018). Mice were placed onto the center platform, and their choices to step down were recorded. Each mouse was subjected to the test once. The box and central platform were thoroughly cleaned after each test.

### Looming Visual Stimulus Response Test

The test for looming visual stimulus response was conducted in an enclosure with dimensions of 17 inches×20 inches×12 inches, built with materials purchased from 80/20 Inc. (Columbia City, IN) as described (Koehler et al., 2019). A 5-inch wide board was placed at one end of the enclosure at the height of 3 inches to act as a hideout. Food pieces were placed at the side opposite the hideout to encourage mice to explore their environment and remain outside of the hideout. A monitor was placed on top of the enclosure to display the looming stimulus, a video of an expanding black disk on a gray background made using Blender software. The stimulus parameters were adapted from a previous study (Yilmaz and Meister, 2013), consisting of a circle expanding from a radius of 2-degrees to 20-degrees in 250 milliseconds, where it remained for 250 milliseconds. The stimulus was displayed 15 times, with a 500-millisecond interval between presentations. An overhead camera recorded mouse behavior. Mice were placed in the enclosure for 10 minutes prior to stimulus onset to allow time to acclimate. Three responses were assessed during the looming stimulus: freezing, fleeing, and tail rattling (Koehler et al., 2019; Lim et al., 2016; Salay et al., 2018; Yilmaz and Meister, 2013). If a mouse demonstrated at least one of these behaviors over the course of the stimulus, it was tallied as a positive looming responder. Each mouse was subjected to the test once. The enclosure was thoroughly cleaned after each test.

### Quantification and Statistical Analysis

Excel and GraphPad Prism 9 were used for statistical analysis. All of the statistical details for each experiment were described in the figure legends.

For behavioral tests where injection and injury treatment was performed bilaterally on both eyes for each mouse, the indicated “n” represents individual mice; for other tests where injection and injury treatment was performed unilaterally on one eye for each mouse, the indicated “n” represents individual eyes and thus only one eye per mouse was used for statistical analysis.

An unpaired t-test was used to compare two groups. One-way ANOVA with Tukey's multiple comparisons test, with follow-up tests to compare each group with every other group, was used to compare multiple groups. Fisher's exact test was used to compare groups in visual cliff test and looming visual stimulus response test. A P-value ≤0.05 was considered statistically significant.

### Results

### 1. Excitotoxic and Axonal Injuries Lead to Loss of CaMKII Activity in RGCs.

CaMKII has four isoforms (α, β, γ, and δ) in mammals, with each isoform expressed from a different gene (Hudmon and Schulman, 2002b). CaMKIIα and CaMKIIβ are the two major isoforms highly expressed in the rodent retina (Terashima et al., 1994). Activation of CaMKII is initiated by Ca²⁺ influx through NMDARs and subsequent Ca²⁺/Calmodulin binding; the resultant conformation change of CaMKII allows its autophosphorylation at either Threonine 286 (T286) for CaMKIIα or Threonine 287 (T287) for CaMKIIβ, which is crucial for the persistent activation of both isoforms (Miller et al., 1988; Schworer et al., 1988; Thiel et al., 1988). If autophosphorylation occurs, CaMKII remains active even after Ca²⁺ concentration falls (Lisman et al., 2002).

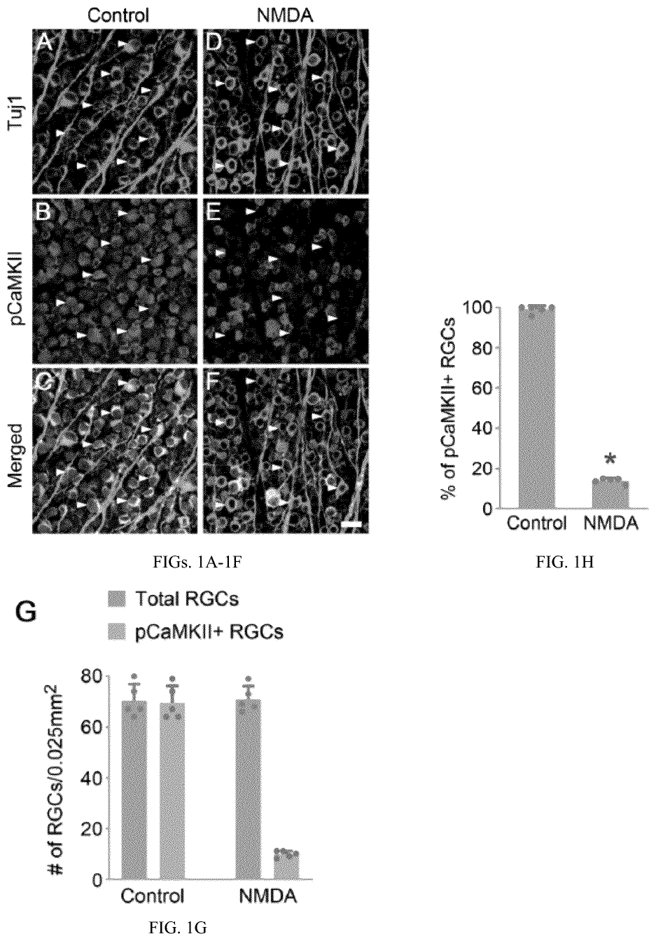

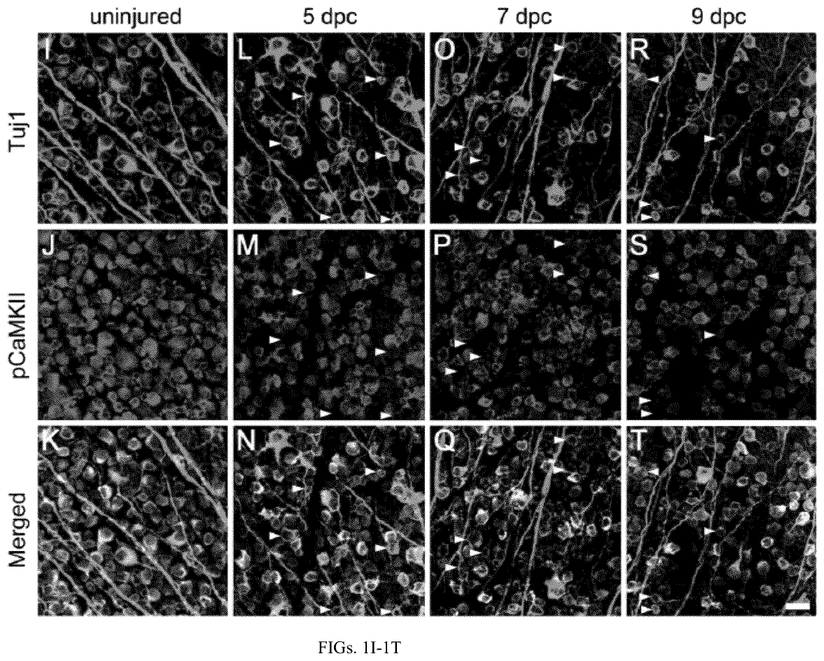

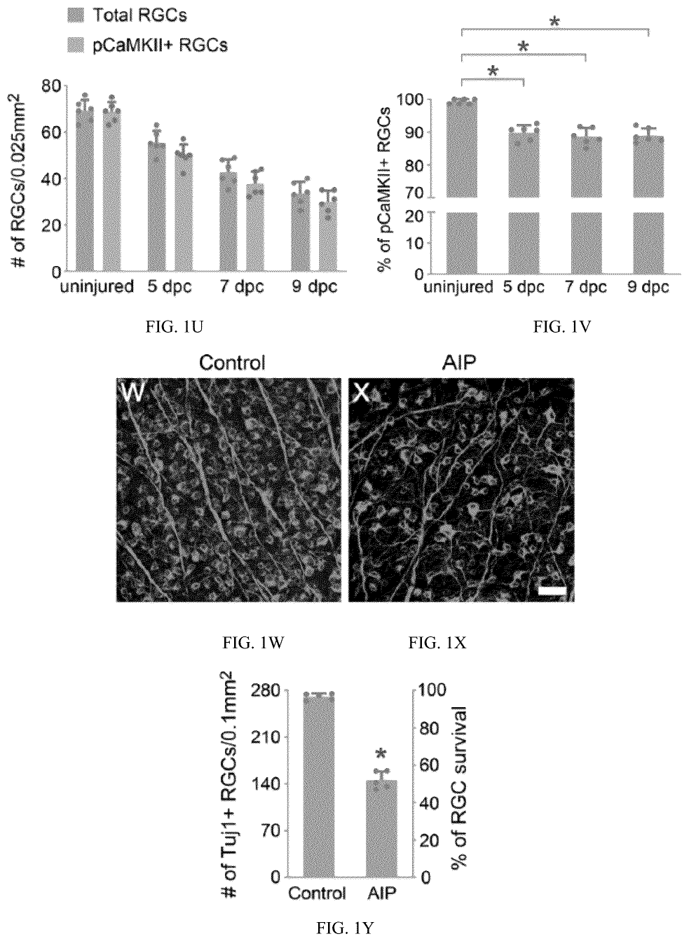

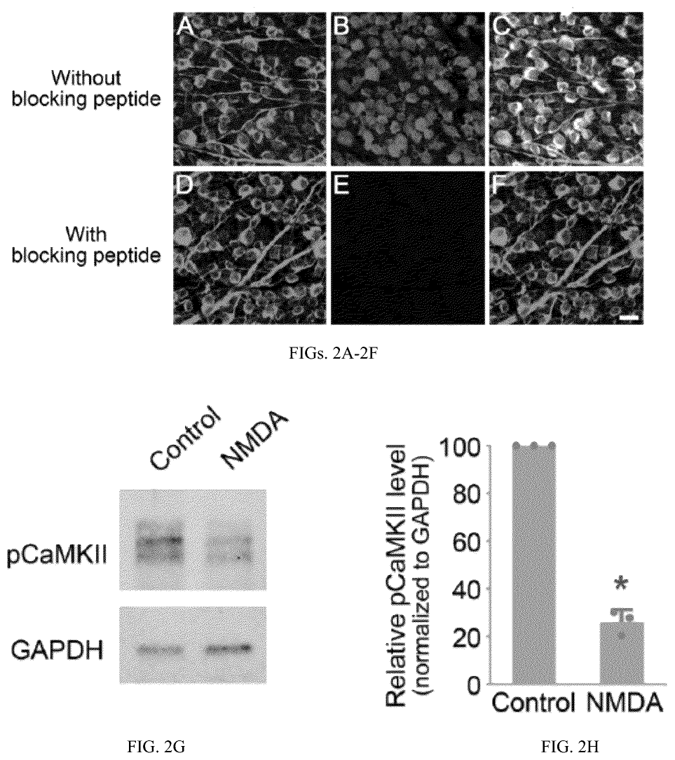

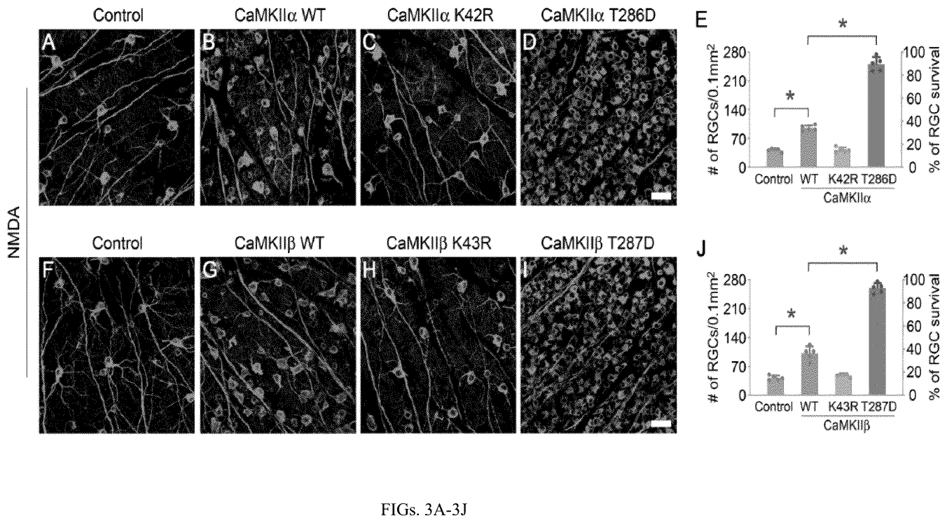

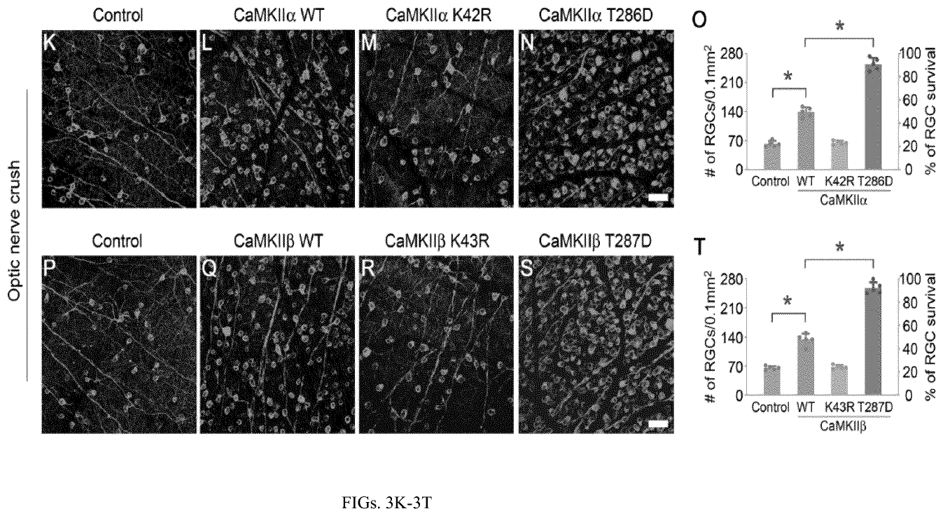

Using an antibody specifically recognizing CaMKIIα/β autophosphorylation (FIGS. 2A-2F), we examined CaMKII phosphorylation in RGCs after retinal injuries. First, we used NMDA-induced excitotoxicity to damage RGC somas after injecting toxic levels of NMDA (20 mM, 1.5 μl) into the vitreous chamber of 8-week-old C57BL/6 mice to injure RGCs (Manabe and Lipton, 2003; Seitz and Tamm, 2013). In the control retina with PBS injection, CaMKII was highly phosphorylated in RGCs labeled by Tuj1 immunoreactivity in retinal flat-mount preparations (FIGS. 1A-1C); whereas in the retina with NMDA injection, CaMKII became massively de-phosphorylated in Tuj1+RGCs 2 hours after injection (FIGS. 1D-1H). Immunoblotting using purified RGCs corroborated the significant decrease in CaMKII phosphorylation after NMDA-induced excitotoxicity (FIGS. 2G and 2H). The loss of CaMKII activity correlated with more than 80% RGC loss at 1 week after NMDA injection (FIGS. 3A and 3E).

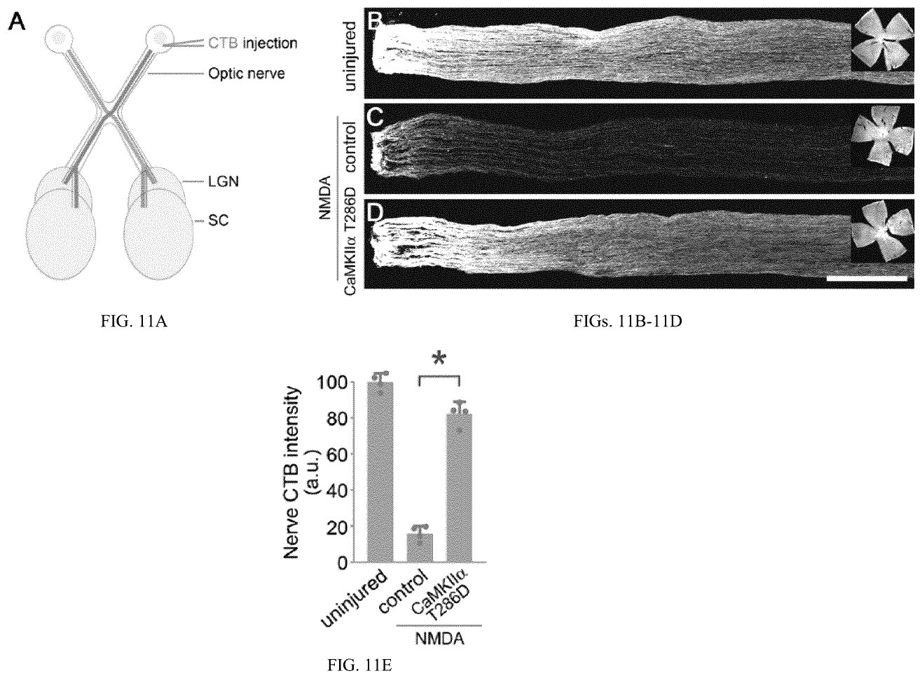

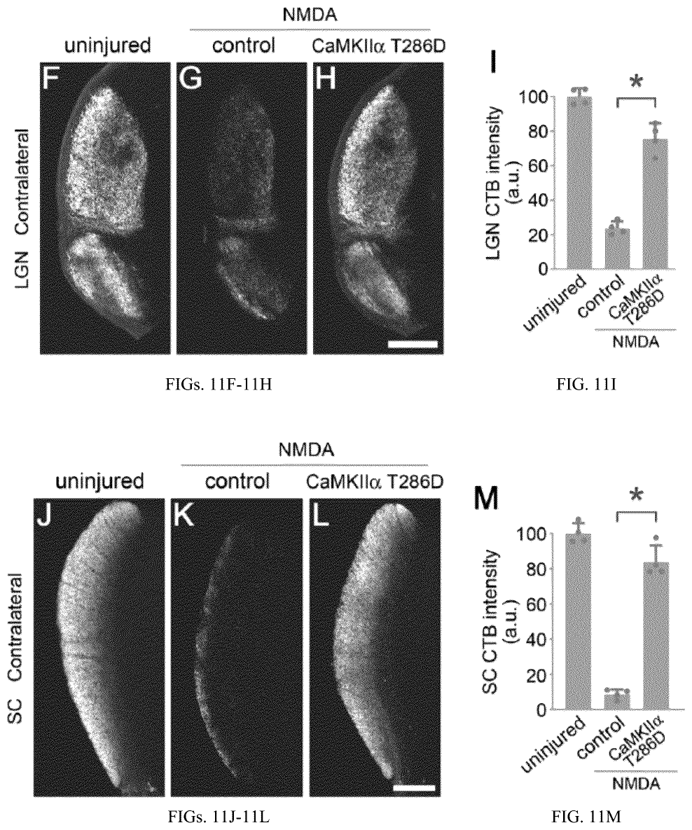

Next, we used the optic nerve crush (ONC) model to examine whether CaMKII activity changes after RGC axonal damage. Axotomy caused by ONC results in delayed death of RGCs, with nearly 80% of RGC loss two weeks after injury (Berkelaar et al., 1994; Hu et al., 2012). We performed ONC in 8-week-old C57BL/6 mice and analyzed CaMKII phosphorylation at 5, 7 and 9 days post crush (dpc) during the course of RGC degeneration. In comparison to the uniform phosphorylation of CaMKII in all RGCs in the uninjured retina (FIGS. 11-1K), at each time point after injury, a portion of RGCs (˜10%) lost CaMKII activity, consistent with the gradual loss of RGCs after axotomy (FIGS. 1L-1V). Overall, these results show that excitotoxicity to RGC somas or optic nerve injury to RGC axons resulted in a loss of CaMKII activity prior to the loss of RGCs.

As CaMKII activity is highly detectable in all RGCs in uninjured retinas (FIGS. 1B and 1J), we investigated whether CaMKII activity is required for normal RGC survival. CaMKII activity was inhibited by daily intravitreal injection (7 injections over 7 days) of myristoylated Autocamtide-2-Related Inhibitory Peptide (AIP, 1 mM, 1.5 μl), a highly potent and specific substrate competitive inhibitor of CaMKII (Goebel, 2009; Laabich and Cooper, 2000). One week after AIP injection, about half of RGCs were lost compared to the vehicle control (FIGS. 1W-1Y). These results indicate that CaMKII activity is essential for the survival of RGCs in the normal retina.

2. Reactivation of CaMKII Protects RGCs from Excitotoxic or Axonal Injuries.

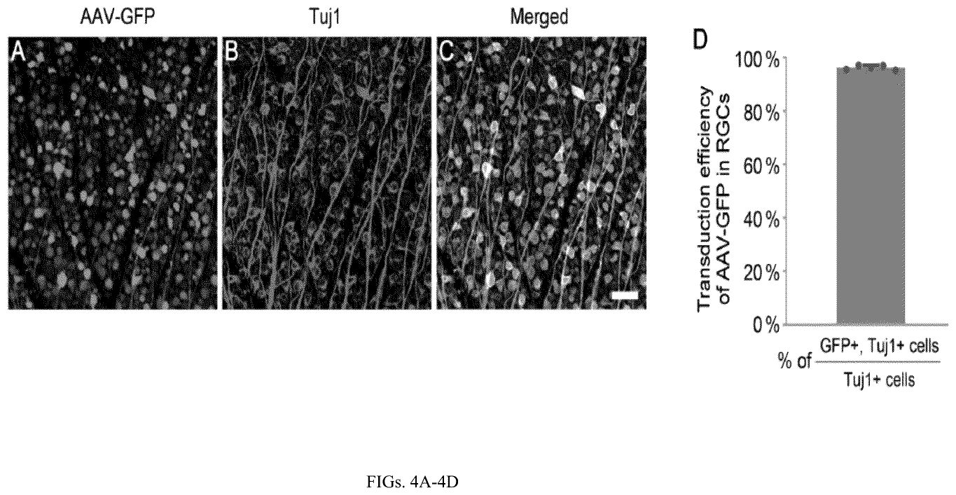

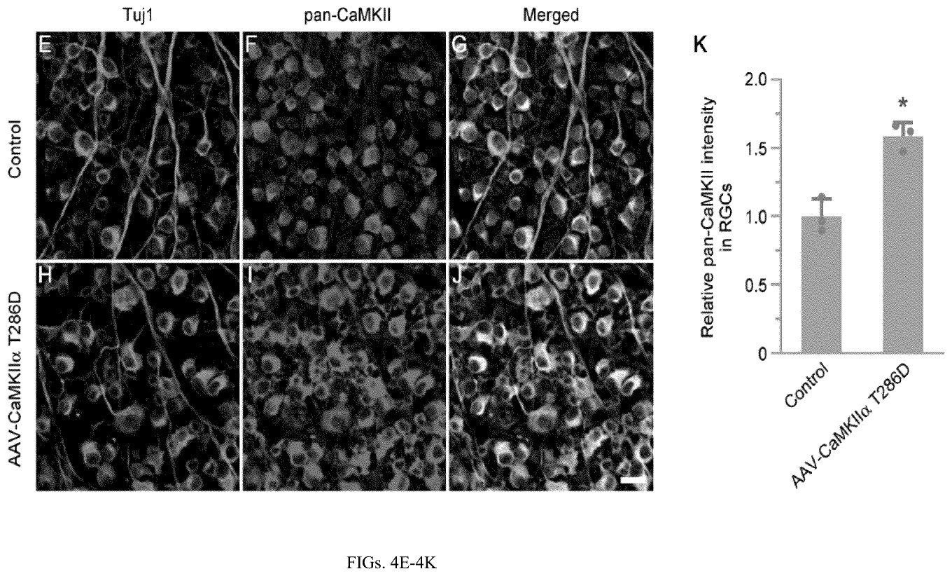

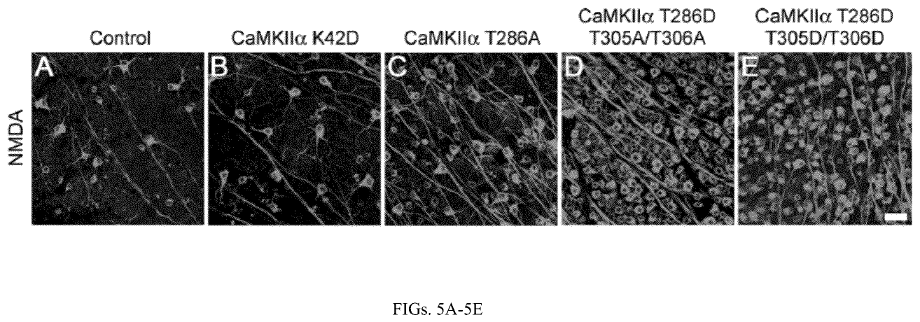

To investigate whether enhancing the activity of CaMKII is sufficient to protect RGCs from excitotoxic or axonal injuries, we performed intravitreal injection for AAV2-mediated gene transfer of CaMKII variants into RGCs in 8-week-old C57BL/6 mice at two weeks before injury onset through NMDA injection or ONC. AAV2 was effective in transducing more than 95% of RGCs (FIGS. 4A-4D) (Park et al., 2008). The expression level of CaMKII variants in RGCs was ˜60% of the endogenous CaMKII based on relative pan-CaMKII immunofluorescence intensity two weeks after AAV injection (FIGS. 4E-4K).

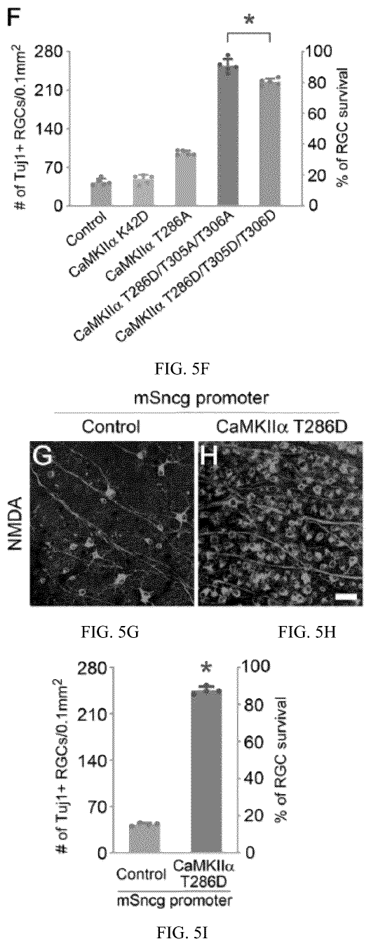

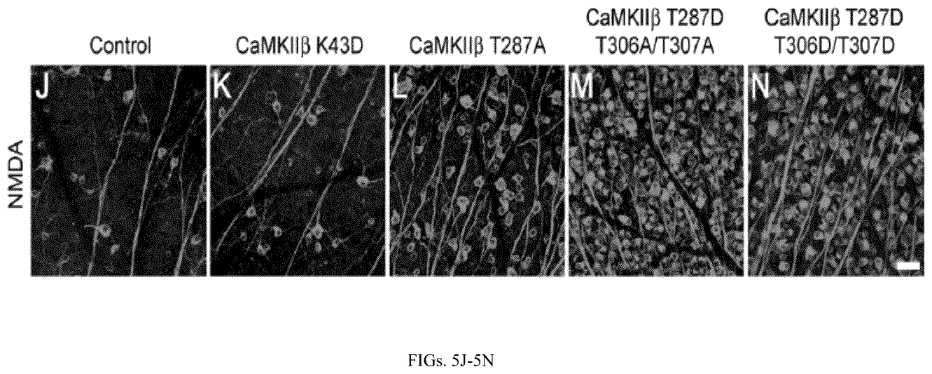

First, we examined the protective effects of CaMKIIα variants when RGC somas were damaged by NMDA-induced excitotoxicity. One week after NMDA administration, the number of Tuj1⁺ RGCs nearly doubled (˜34%) in the wild-type CaMKIIα-treated retinas compared to the control group injected with AAV2-EBFP, in which case only ˜15% of RGCs remained (FIGS. 3A, 3B, and 3E). To examine whether the kinase activity is essential for CaMKIIα-mediated RGC protection, we tested CaMKIIα K42R and CaMKIIα K42D, two kinase-dead mutants by replacing the conserved Lysine residue with Arginine or Aspartic acid within the catalytic core of the enzyme to prevent ATP-binding necessary for its kinase activity (Hanson et al., 1994). We observed no improvement of RGC survival with either CaMKIIα K42R (FIGS. 3A, 3C, and 3E) or CaMKIIα K42D treatment (FIGS. 5A, 5B, and 5F), indicating that CaMKII kinase activity is required for protecting RGCs from NMDA-induced excitotoxic RGC death.

To examine whether further enhancement of CaMKII activity could be more effective in countering excitotoxic injury, we tested CaMKIIα T286D, a constitutively active mutant of CaMKIIα simulating its autophosphorylated state (Fong et al., 1989). Remarkably, CaMKIIα T286D robustly protected the vast majority of RGCs (˜90%) at 1 week after NMDA injection compared to a small proportion of surviving RGCs (˜15%) in the control group (FIGS. 3A, 3D, and 3E). However, the autophosphorylation-defective mutant CaMKIIα T286A (Fong et al., 1989) was far less effective than CaMKIIα T286D in protecting RGCs (FIGS. 55A and 5F).

In addition to autophosphorylation of CaMKIIα at T286, phosphorylation at T305 and T306 within the calmodulin-binding domain also regulates its kinase activity. Following T286 phosphorylation and Ca²⁺/CaM dissociation, autophosphorylation of CaMKIIα at T305 and T306 abolishes its sensitivity to Calmodulin binding and thus prevents its further activation (Coultrap et al., 2010; Patton et al., 1990). In hippocampal neurons, phosphorylation at T305 and T306 participates in CaMKIIα T286D-mediated control of the spine size and synaptic strength, as well as long-term potentiation (LTP) or long-term depression (LTD) induction (Pi et al., 2010a; Pi et al., 2010b). To examine whether further phosphorylation of CaMKIIα at T305 and T306 affects RGC survival, we tested the treatment effects of CaMKIIα T286D/T305A/T306A (a non-phosphorylated form of T305/T306) and CaMKIIα T286D/T305D/T306D (a pseudophosphorylated form of T305/T306) after excitotoxic injury to RGC somas. CaMKIIα T286D/T305A/T306A protected ˜90% of RGCs at 1 week after NMDA injection (FIGS. 5D and 5F); whereas CaMKIIα T286D/T305D/T306D protected ˜80% of RGCs, slightly less effective in protecting RGCs compared to T286D/T305A/T306A (FIGS. 5E and 5F). These results suggest that phosphorylation at T305 and T306 may interfere modestly with the maximum effects of CaMKIIα T286D in protecting RGCs.

The ubiquitous CAG promoter in our AAV2 expression system drives transgene expression in more than 95% of RGCs as well as a few other non-RGC cells (Park et al., 2008). To examine whether CaMKII-mediated RGC protection was indeed due to transgene expression in a cell-autonomous manner, we used the mouse γ-synuclein promoter (mSncg), a recently developed RGC-specific promoter that drives AAV2-mediated transgene expression in mouse RGCs (Wang et al., 2020), to restrict CaMKII expression to RGCs. One week after NMDA injection, AAV2-mSncg-mediated expression of CaMKIIα T286D protected RGCs as effectively as that mediated by AAV2-CAG (FIGS. 5G-5I), indicating that cell-autonomous expression of CaMKIIα T286D in RGCs is essential for protecting them from NMDA-induced excitotoxicity.

As CaMKIIβ is the other major isoform expressed in the mouse retina, we examined the protective effects of CaMKIIβ variants after NMDA-induced excitotoxicity. Similar to the protective effects obtained from CaMKIIα treatment, the wild-type CaMKIIβ showed a moderate protective effect, while the constitutively active mutant CaMKIIβ T287D was far more potent in protecting RGCs a week after NMDA injection (FIGS. 3F, 3G, 3I, and 3J). As expected, the autophosphorylation-defective mutant CaMKIIβ T287A protected many fewer RGCs relative to CaMKIIβ T287D (FIGS. 5L and 50). Phosphorylation at T306 and T307 within the calmodulin-binding domain also slightly reduced the protective effects of CaMKIIβ T287D, evidenced by treatment effects with the triple mutants CaMKIIβ T287D/T306A/T307A or CaMKIIβ T287D/T306D/T307D (Figures S3M-S3O). CaMKIIβ-mediated protection of RGCs apparently also relied on its kinase activity as the kinase-dead mutant CaMKIIβ K43R (FIGS. 3H and 3J) or CaMKIIβ K43D (FIGS. 5K and 5O) failed to protect RGCs under the same experimental conditions.

Next, we examined the protective effects of CaMKIIα variants when RGC axons were damaged by ONC. Two weeks after ONC, the number of surviving RGCs (˜49%) was more than doubled with the wild-type CaMKIIα treatment compared to surviving RGCs (˜22%) in the control group injected with AAV-EBFP (FIGS. 3K, 3L, and 3O). However, the kinase-dead mutant CaMKIIα K42R showed no protection (FIGS. 3M and 3O), indicating that the kinase activity of CaMKII is also required to protect RGCs from axotomy-induced cell death. Remarkably, the constitutively active mutant CaMKIIα T286D exhibited the most effective protective effect, with ˜90% of RGCs surviving two weeks after injury (FIGS. 3N and 3O). As expected, CaMKIIβ variants exhibited similar protective effects as their CaMKIIα counterparts, confirming the necessity for its kinase activity and the remarkable protective effect when CaMKIIβ activity was further enhanced with the constitutively active mutant CaMKIIβ T287D (FIGS. 3P-3T).

Using two injury models, our results demonstrate that excitotoxic damage to RGC somas or ONC damage to RGC axons inevitably leads to CaMKII inactivation prior to RGC death. Reactivation of CaMKII via gene transfer of constitutively active CaMKII mutants robustly protects RGCs from both injuries. Since CaMKIIα T286D manifested the most robust protection for RGCs, it became the focus of our subsequent studies.

### 3. CaMKII Reactivation Provides Post-Injury and Long-Term Protection of RGCs.

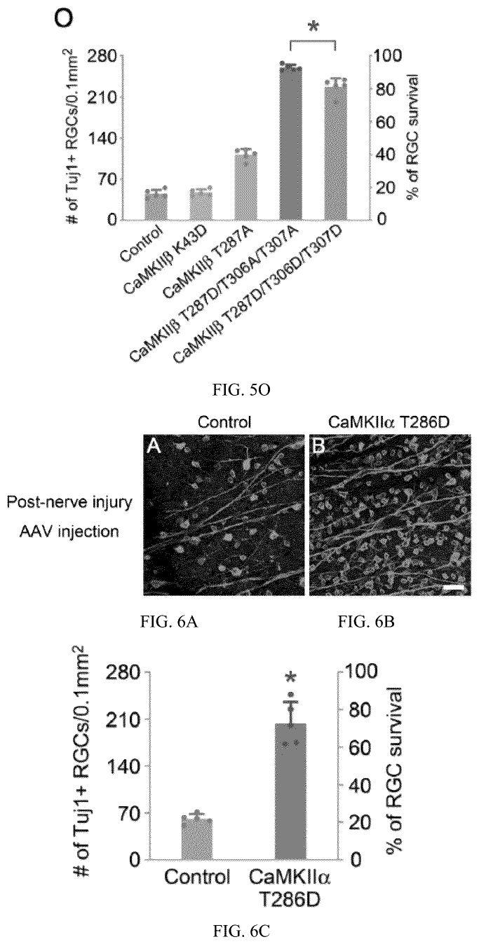

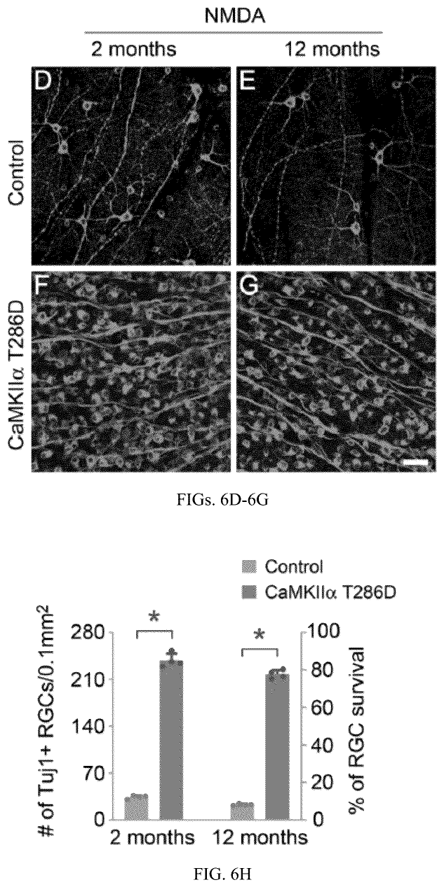

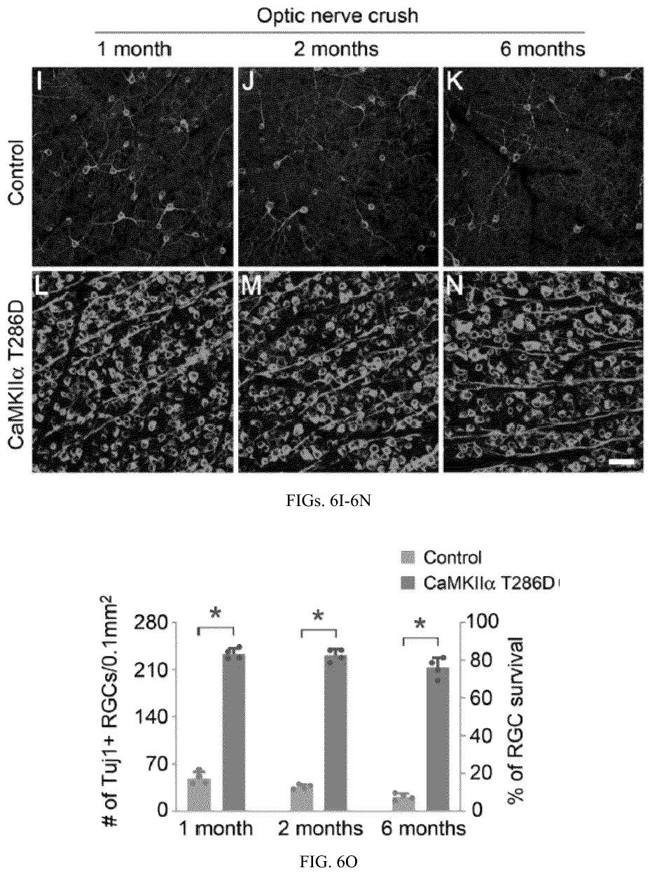

To mimic a clinically relevant setting, we tested whether CaMKII could protect RGCs when reactivated following the injury. While RGC degeneration in the excitotoxicity model is too fast for AAV-mediated gene expression to take effect, the relatively slower degeneration of RGCs in the optic nerve crush model may allow a time window necessary for AAV-mediated gene therapy (Sun et al., 2011). Therefore, we performed intravitreal AAV injections immediately after ONC. Two weeks later, ˜70% of RGCs remained in the CaMKIIα T286D treatment group, tripling the survival rate relative to the control group receiving AAV-mediated gene transfer of EBFP (FIGS. 6A-6C). These results indicate that delayed reactivation of CaMKII may robustly protect RGCs after injury onset.

CaMKII reactivation protected the vast majority of RGCs 1 week after NMDA-induced excitotoxicity or 2 weeks after ONC. To evaluate the long-term protective effects of CaMKIIα T286D treatment, we assayed RGC survival at a much later time after excitotoxic or axonal injuries. After the excitotoxic injury to RGC somas, RGC numbers in the control group receiving AAV-EBFP continued to decline from ˜12% at 2 months (FIG. 6D) to ˜8% RGCs remaining at 12 months (FIG. 6E). Remarkably, CaMKIIα T286D treatment resulted in ˜84% and ˜77% of RGCs surviving 2 and 12 months after injury, respectively (FIG. 6F-6H). Next, we examined the long-term RGC survival after ONC to injure RGC axons. While only ˜17%, ˜12%, and ˜7% of RGCs remained from the control group receiving AAV-EBFP at 1, 2, and 6 months after ONC (FIGS. 6I-6K and 6O), CaMKIIα T286D treatment protected ˜82%, ˜81%, and ˜77% of RGCs (FIGS. 6L-6O) at the same time points. Taken together, these results demonstrate that CaMKII reactivation may provide long-term protection of RGCs from excitotoxic or axonal injuries.

### 4. CREB Acts Downstream of CaMKII in Protecting RGCs.

To delineate the downstream signaling of CaMKII-mediated RGC protection, CREB (cAMP response element binding protein), a stimulus-induced transcription factor, was of particular interest because CREB plays an important role downstream of CaMKII in regulating synaptic plasticity and long-term memory formation (Deisseroth et al., 1996; Ma et al., 2014). CREB is also known to regulate the survival of brain neurons (Lonze and Ginty, 2002). However, the role of CREB in regulating the survival of retinal neurons remains to be determined. CREB is phosphorylated at Ser133 by CaMKII and other kinases, and the phosphorylation event is required for the transcriptional activity of CREB (Sheng et al., 1991).

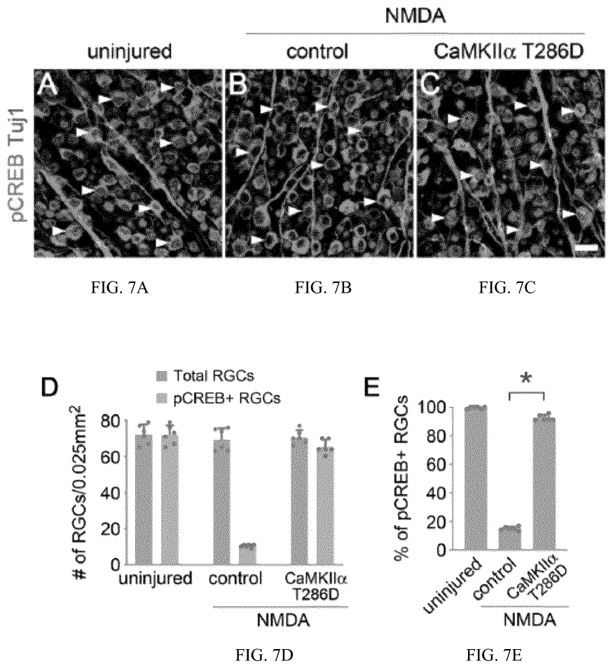

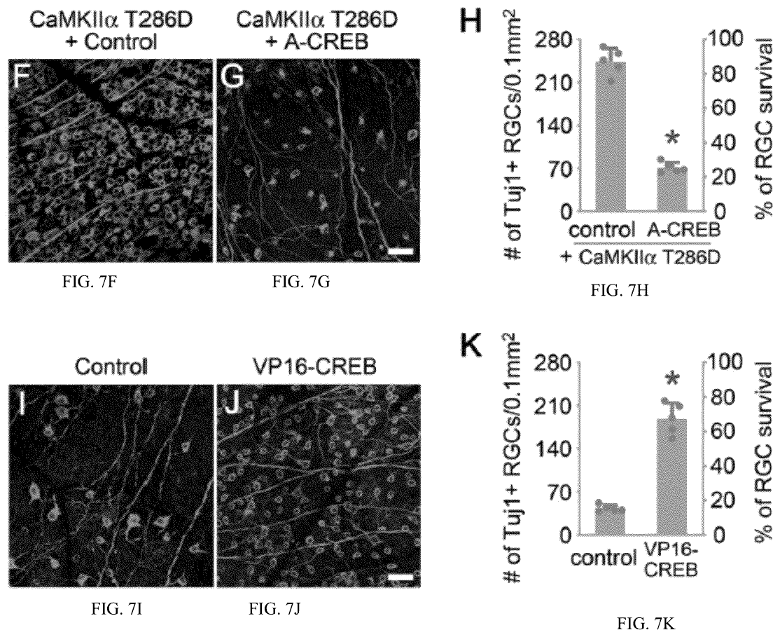

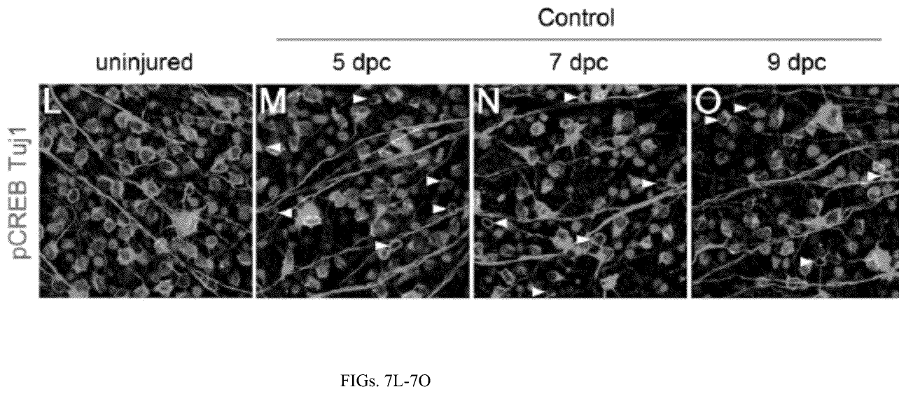

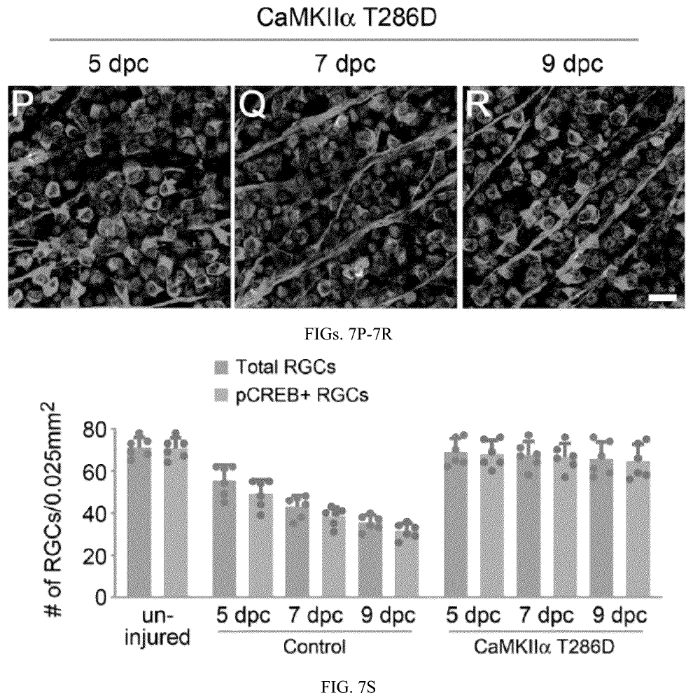

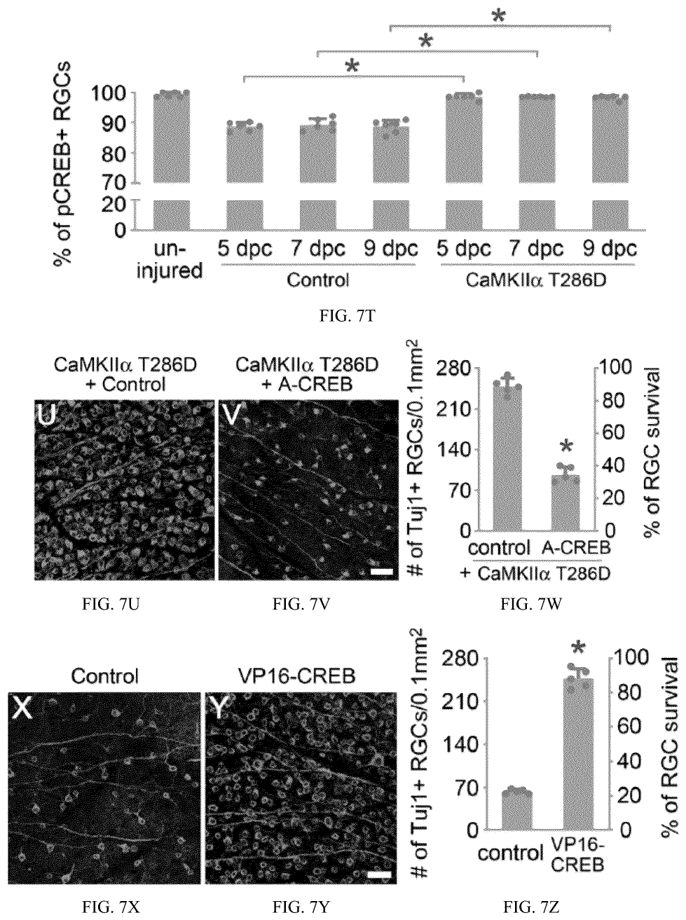

First, we examined whether CREB acts downstream of CaMKII to protect RGCs in the NMDA-induced excitotoxicity model. Using an antibody recognizing phosphorylated CREB at Ser133, we found that CREB was highly phosphorylated in nearly all Tuj1+RGCs in uninjured retinas (FIGS. 7A, 7D, and 7E). However, a massive loss of CREB phosphorylation occurred in RGCs 2 hours after NMDA injection (FIGS. 7B, 7D, and 7E), indicating that compromised CREB activity may be downstream of CaMKII inactivation in RGCs after excitotoxic injury. Then we examined whether CaMKII reactivation could restore CREB activity following NMDA injection. Indeed, AAV2-mediated gene transfer of CaMKIIα T286D was sufficient to maintain CREB phosphorylation in nearly all RGCs exposed to NMDA insults (FIGS. 7C, 7D, and 7E).

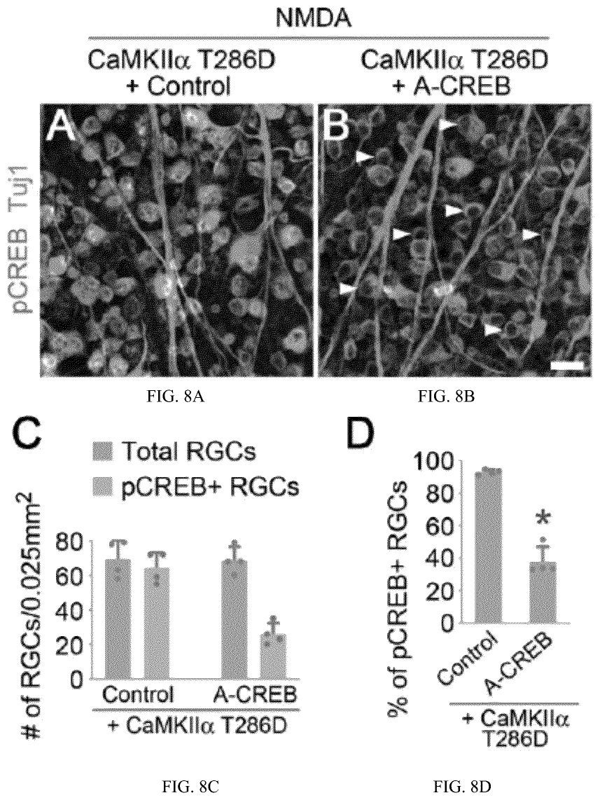

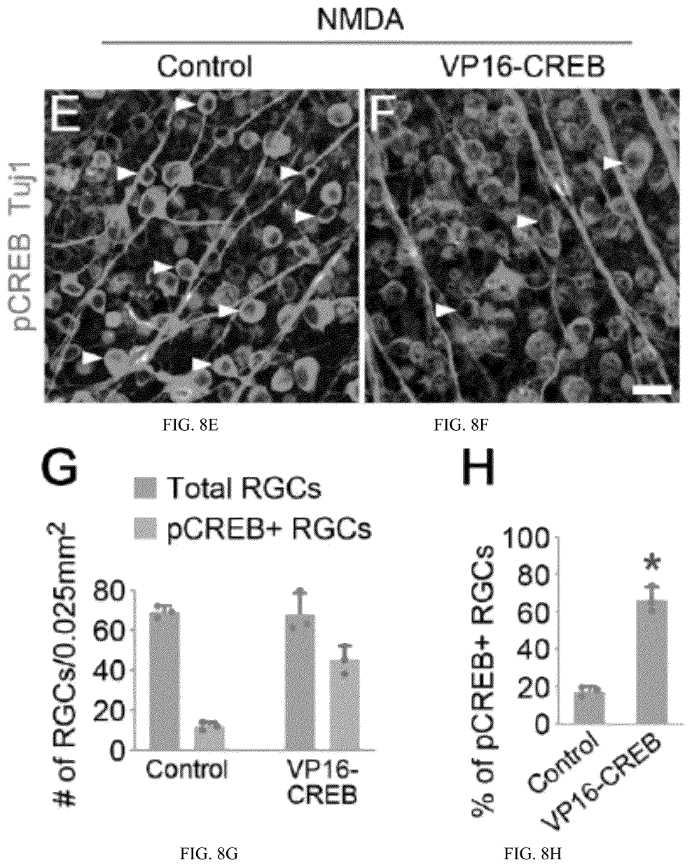

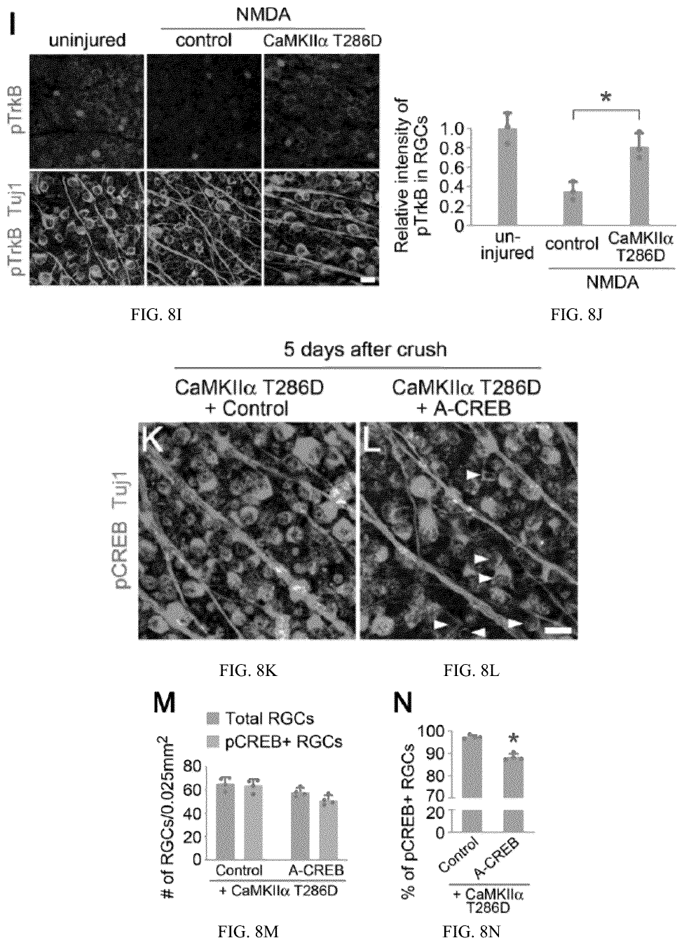

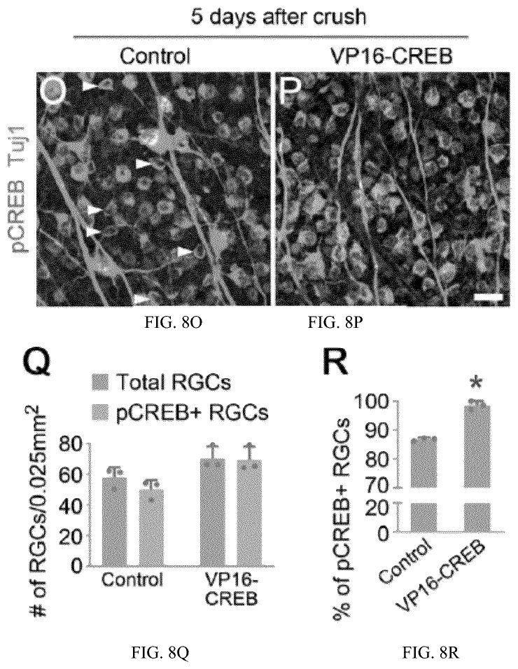

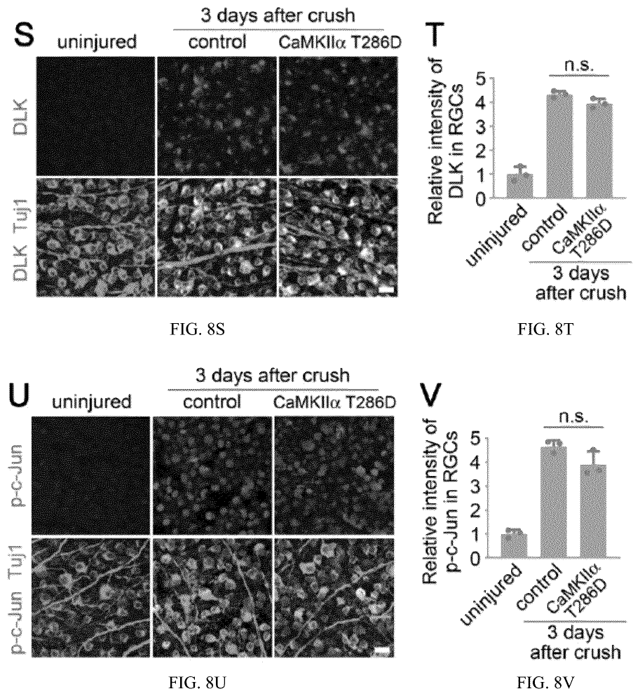

To further investigate the role of CREB in protecting RGCs downstream of CaMKII, we performed AAV2-mediated gene transfer of CaMKIIα T286D together with A-CREB, a dominant negative variant of CREB that binds to endogenous CREB protein and prevents CREB from binding to DNA (Ahn et al., 1998). AAV2 delivery was performed in 8-week-old C57BL/6 mice at 2 weeks before NMDA injection, and we analyzed RGC survival 1 week after excitotoxic injury. CaMKIIα T286D-mediated RGC protection was nearly neutralized with A-CREB co-treatment (FIGS. 7F-7H), indicating that CREB activation is required for CaMKII-mediated RGC protection from excitotoxic damage. Indeed, A-CREB co-treatment significantly compromised CREB phosphorylation by CaMKIIα T286D in RGCs 2 hours after NMDA injection (FIGS. 8A-8D). To test whether activation of CREB alone, independent of CaMKII activation, is sufficient to protect RGCs from excitotoxicity, we performed AAV2-mediated gene transfer of VP16-CREB, a constitutively active variant of CREB (Barco et al., 2002) at 2 weeks before NMDA injection, and analyzed RGC survival 1 week after injury. Evidently, VP16-CREB treatment alone protected a majority of RGCs (˜65%) (FIGS. 7I-7K). Consistent with a similar role in the hippocampus (Zhang et al., 2016), VP16-CREB maintained CREB phosphorylation in RGCs 2 hours after NMDA injection (FIGS. 8E-8H). The protective effect of VP16-CREB (188.7±25.4 RGCs/0.1 mm²) was weaker than that of CaMKIIα T286D (251.2±16.9 RGCs/0.1 mm²), suggesting that there might be other unidentified factors downstream of CaMKII in protecting RGCs from excitotoxicity.

BDNF (Brain-derived neurotrophic factor)/TrkB (tropomyosin-related kinase receptor type B) signaling contributes to neuroprotection of RGCs, and excitotoxicity downregulates this pathway (Chitranshi et al., 2019; Gomes et al., 2012). To investigate whether CaMKIIα T286D treatment affects BDNF/TrkB signaling, we examined TrkB phosphorylation, which is essential for the downstream signal transduction initiated by BDNF binding (Nagahara and Tuszynski, 2011). Phosphorylation of TrkB at Tyr817 was readily detectable in RGCs of the uninjured retina; NMDA insult resulted in a significant reduction of phospho-TrkB in RGCs, which was rescued by CaMKIIα T286D treatment (FIGS. 81 and 8J). These results indicate that, in addition to CREB, CaMKII reactivation may affect other pathways such as BDNF/TrkB signaling in promoting RGC survival after excitotoxic injury.

Next, we examined whether CREB acts downstream of CaMKII to protect RGCs in the ONC-induced axonal injury model. We analyzed CREB activity using the antibody recognizing CREB phosphorylation at Ser133 at 5, 7, and 9 days after ONC. In comparison to uniform CREB phosphorylation in RGCs of the uninjured retina (FIG. 7L), a proportion (˜10%) of RGCs lost CREB phosphorylation at each time point examined (FIGS. 7M-7O, 7S, and 7T). The progressive loss of CREB activity is consistent with the gradual loss of CaMKII activity after ONC (FIGS. 1L-1V), indicating that compromised CREB activity may be downstream of CaMKII inactivation after RGC axonal injury. Next, we examined whether CaMKII reactivation could rescue CREB phosphorylation after ONC. Indeed, AAV-mediated gene transfer of CaMKIIα T286D maintained CREB phosphorylation in nearly all Tuj1⁺ RGCs after injury (FIGS. 7P-7T). Similar to the observation in the excitotoxicity model, CREB transcriptional activity was necessary for CaMKII-mediated RGC protection 2 weeks after RGC axonal injury, as the dominant negative variant A-CREB neutralized the protective effects mediated by CaMKIIα T286D (FIGS. 7U-7W). As expected, A-CREB co-treatment significantly compromised CaMKIIα T286D-mediated CREB phosphorylation in RGCs 5 days after ONC (FIGS. 8K-8N). Conversely, enhancing CREB activity alone by gene transfer of the constitutively active variant VP16-CREB maintained CREB phosphorylation in RGCs 5 days after ONC (FIGS. 8O-8R) and efficiently protected RGCs from axonal injury (FIGS. 7X-7Z).

The DLK (dual leucine zipper kinase)/c-Jun pathway is a prominent mediator of RGC cell death after optic nerve injury (Watkins et al., 2013; Welsbie et al., 2013). To investigate whether CaMKIIα T286D treatment modulates the DLK/c-Jun pathway, we examined DLK expression and c-Jun phosphorylation at Serine 63. As previously reported, DLK and c-Jun phosphorylation levels were very low in uninjured retinas and dramatically upregulated 3 days after ONC. However, CaMKIIα T286D treatment did not affect these changes (FIGS. 8S-8V), indicating that CaMKIIα-mediated RGC protection from optic nerve injury may not act by suppressing the DLK/c-Jun pathway.

Taken together, our results demonstrate that CREB is a key downstream effector of CaMKII as it is both necessary and sufficient in protecting RGCs from excitotoxicity to their somas or a crush injury to their axons.

### 5. CaMKII-Mediated Protection of RGCs in Induced and Genetic Models of Glaucoma.

Glaucoma, characterized by the progressive degeneration of RGC axons with subsequent loss of the respective somas, is a leading cause of irreversible blindness worldwide. The pathogenesis of glaucoma is not well understood, but it is typically associated with elevated intraocular pressure (IOP), which leads to RGC axonal damage and the resultant death of RGCs (Calkins, 2012; Nickells et al., 2012; Weinreb et al., 2016).

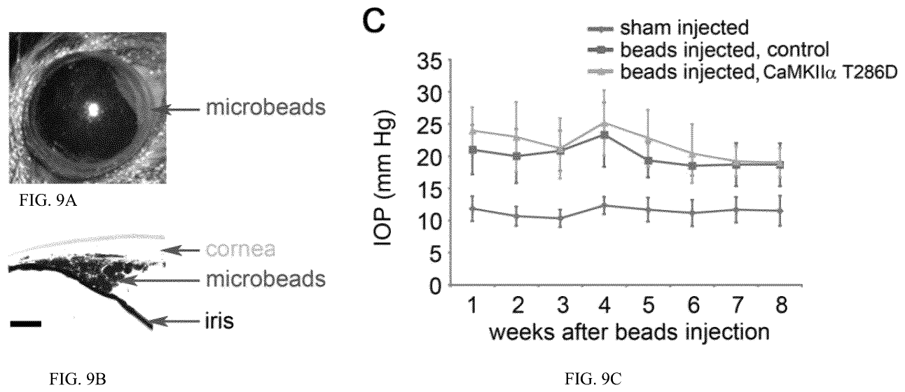

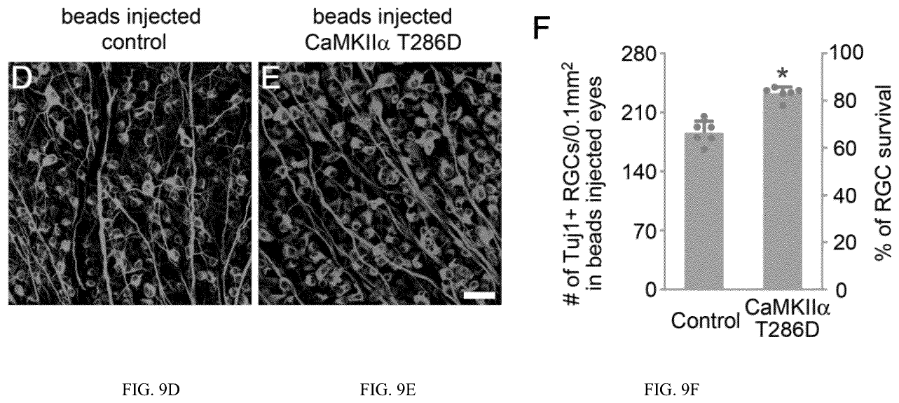

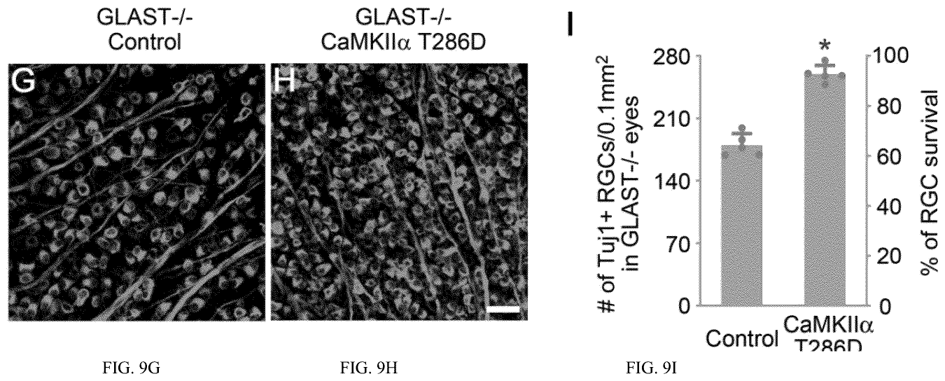

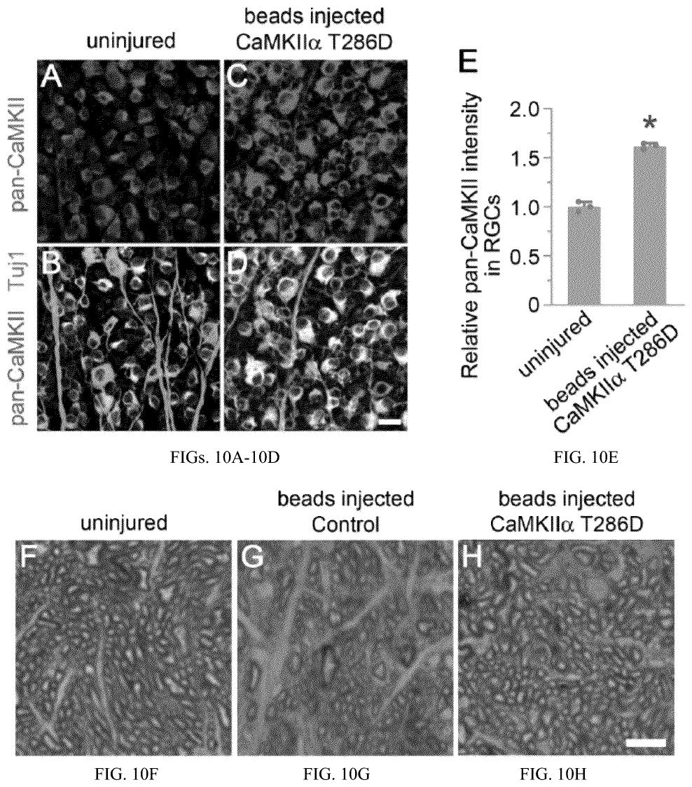

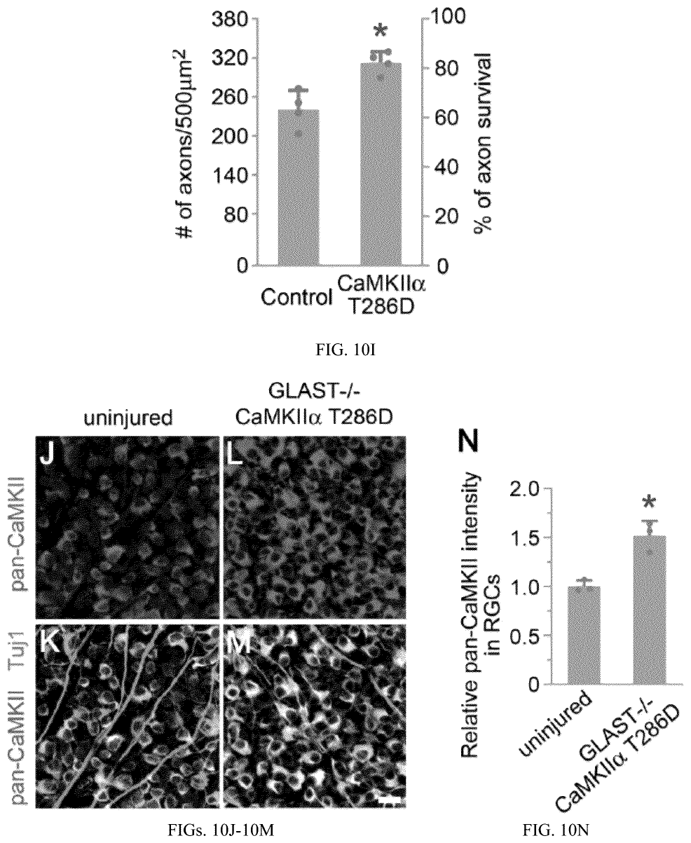

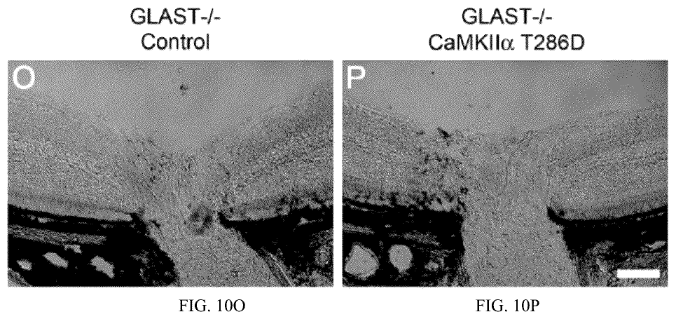

We first tested whether gene transfer of CaMKIIα T286D protects RGCs in a mouse model of hypertension-dependent glaucoma. To induce ocular hypertension, we injected magnetic microbeads into the anterior chamber to occlude aqueous outflow (FIGS. 9A and 9B) (Ito et al., 2016). As a result, IOP showed a sustained elevation for the subsequent eight weeks (FIG. 9C). We performed intravitreal injection of AAV for either the treatment of CaMKIIα T286D or EBFP as a control 2 weeks prior to microbeads injection, and analyzed RGC survival 8 weeks following the induction of ocular hypertension. CaMKII expression levels increased ˜60% based on relative pan-CaMKII immunofluorescence intensity at 2 weeks after microbeads injection (FIGS. 10A-10E). In comparison to ˜66% of surviving RGCs in the control group, CaMKIIα T286D treatment protected ˜82% of RGCs (FIGS. 9D-9F), indicating that CaMKII augmentation is effective at protecting RGCs when they sustain ongoing damage from elevated IOP. We also examined axon survival in optic nerve sections collected at 1 mm behind the eyeball (Yang et al., 2016) and found that CaMKIIα T286D treatment provided significant protection of RGC axons (FIGS. 10F-10I).

Although elevated IOP is the best-known risk factor for glaucoma, a significant number of patients develop the disease despite normal pressure. Mice deficient in the glutamate transporter Glast (GLAST−/−), a model of normal tension glaucoma (Harada et al., 2007), show characteristic degeneration of RGCs due to accumulation of glutamate in the extracellular fluid, as well as increased oxidative stress. We tested whether gene transfer of CaMKIIα T286D protects RGCs in the normal tension glaucoma model of GLAST−/− mice. The onset of RGC death in GLAST deficient mice starts at approximately postnatal day 7. Thus we performed intravitreal AAV injection for CaMKIIα T286D treatment at postnatal day 1 and analyzed the animals at 2 months of age when the degeneration of RGCs has stabilized (Harada et al., 2007). CaMKII expression levels increased ˜50% based on relative pan-CaMKII immunofluorescence intensity at 3 weeks after AAV injection (FIGS. 10J-ION). Significantly, CaMKIIα, T286D treatment protected greater than 90% of RGCs compared to ˜65% remaining in the control group (FIGS. 9G-9I). Consistent with RGC loss, optic nerve degeneration and cupping became apparent in 8-month-old GLAST−/− mice (Harada et al., 2007). CaMKIIα, T286D treatment also alleviated the optic nerve head depletion of RGC axons in these mice (FIGS. 10O and 10P).

Taken together, our results show that CaMKII could be a valuable therapeutic target to slow down the disease progression of glaucoma.

6. CaMKII Reactivation Protects RGC Axons and their Projections in the Brain.

RGC axons are the sole pathway transmitting visual information from the retina to the brain. As RGC axons are rarely able to regenerate after damage, degeneration of RGC axons results in permanent vision loss (Tran et al., 2019). Therefore, protecting the integrity of RGC axons is critical for vision preservation. Although we did not expect that CaMKII reactivation would make RGC axons resistant against severe mechanical damages such as those inflicted by optic nerve crush, it is pivotal to examine whether CaMKII treatment protects RGC axons from pathophysiological insults such as excitotoxicity. Indeed, in addition to damaging RGC somas, excitotoxic insults lead to Wallerian-like degeneration of RGC axons in the optic nerve and loss of target innervation in the brain (Saggu et al., 2010).

To investigate whether reactivation of CaMKII protects RGC axons and their axonal projections to the brain, we injected Alexa Fluor 488-Conjugated Cholera Toxin Subunit B (CTB) into the vitreous to anterogradely trace RGC axons to the lateral geniculate nucleus (LGN) and Superior Colliculus (SC), two main projection targets of RGC axons in the brain (FIG. 11A). One week after NMDA injection, RGC axons were severely damaged, and the CTB labeling intensity decreased to ˜17% in the optic nerve compared to the uninjured control (FIGS. 11B, 11C, and 11E). By contrast, a significant majority of axons were protected from excitotoxicity after CaMKIIα, T286D treatment with the CTB labeling intensity recovered to ˜84% (FIGS. 11D and 11E).

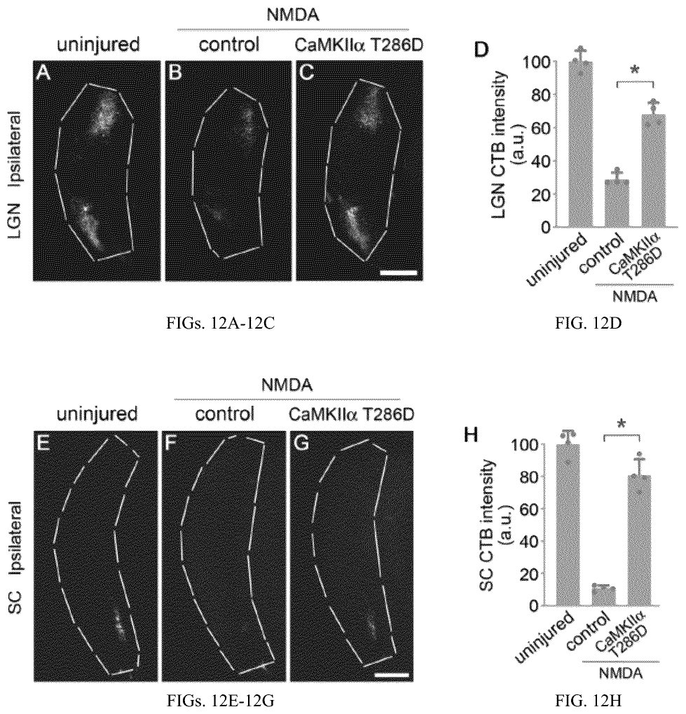

In mice, RGC axons predominantly project to the contralateral hemisphere (Herrera and Mason, 2007). Severe loss of RGC axonal projections in their brain targets was observed 1 week after NMDA injection, with only ˜24% and ˜9% of the CTB labeling intensity remaining in the contralateral LGN (FIGS. 11F, 11G, and 11I) and SC (FIGS. 11J, 11K, and 11M), respectively. By contrast, CaMKIIα T286D treatment protected the majority of RGC axonal projections to the contralateral LGN (˜73%, FIGS. 6H and 61) and SC (˜85%, FIGS. 11L and 11M). There was a similar protection of RGC axonal projections to the ipsilateral LGN (FIGS. 12A-12D) and SC (FIGS. 12E-12H). Collectively, our results demonstrate that CaMKII reactivation not only protects RGC somas, but also robustly preserves the integrity of RGC axons in the optic nerve and their distal projection targets in the brain, a necessity for ultimately preserving functional vision.

### 7. CaMKII Reactivation Preserves Visual Function.

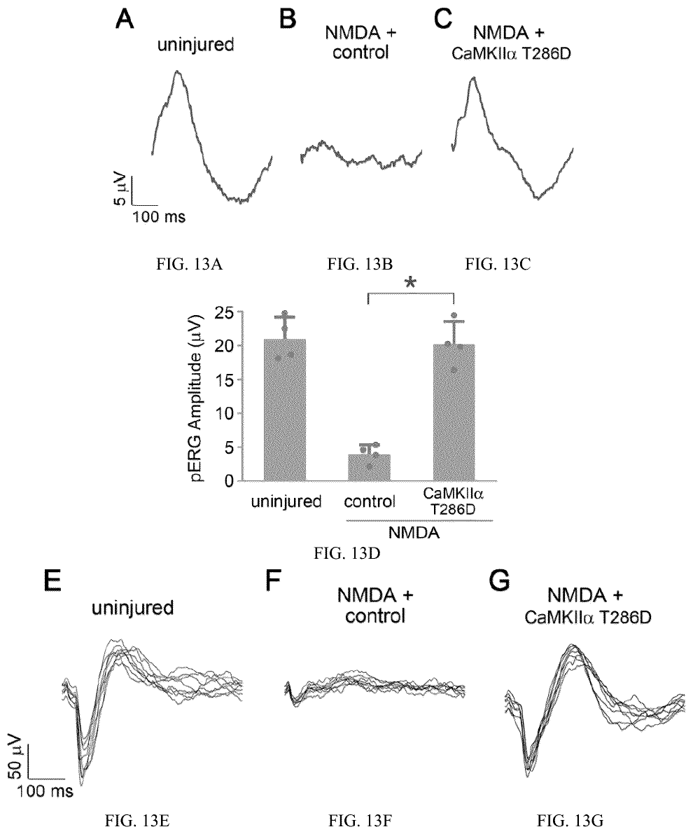

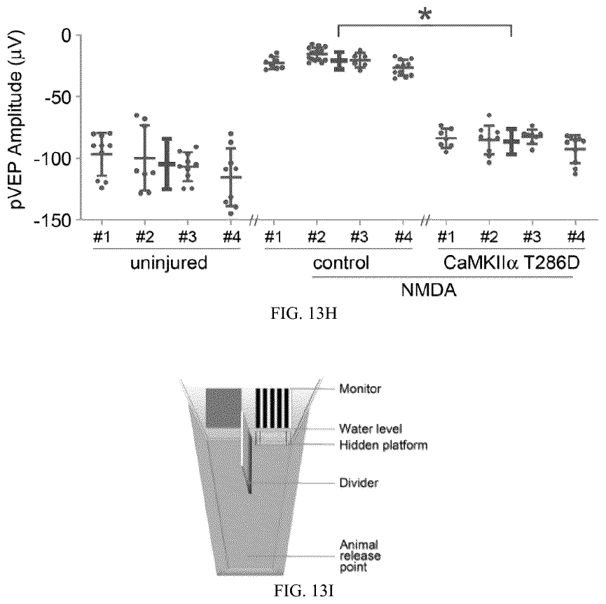

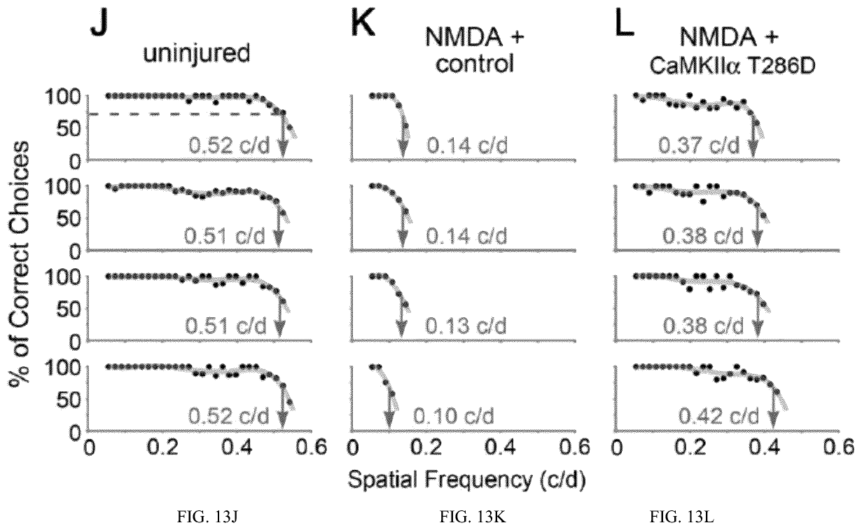

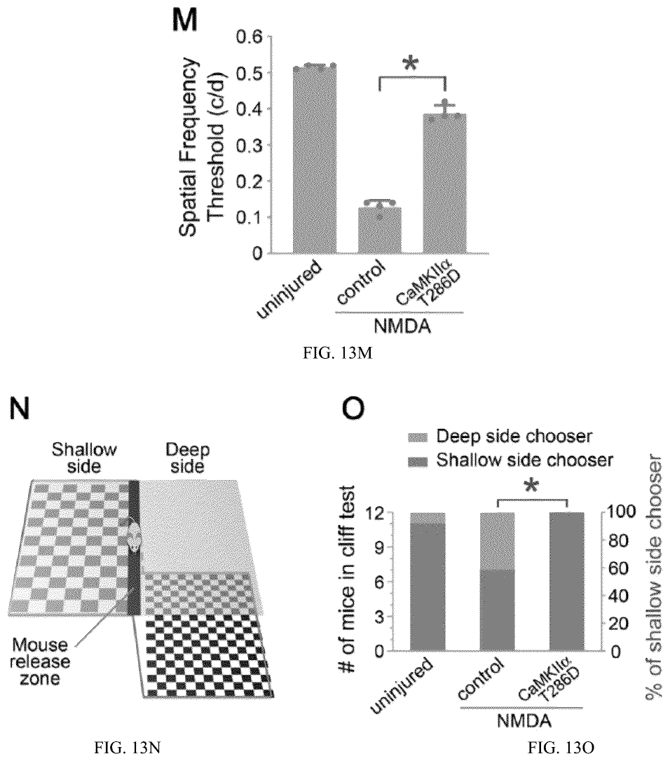

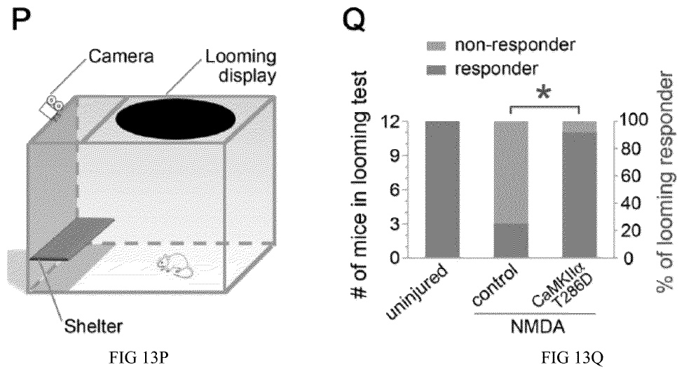

To evaluate whether CaMKII-mediated protection of RGCs from excitotoxicity can preserve vision, first tested was whether treatment of CaMKIIα T286D maintains RGC function using pattern electroretinogram (PERG), which measures RGC activity in response to contrast modulation of patterned visual stimuli (Porciatti, 2007). PERGs were derived simultaneously from each eye in mice using binocular stimulation and a common snout electrode (Chou et al., 2014). PERG responses (21.4 μV) were readily detectable in uninjured retinas (FIG. 13A), which were significantly reduced 7 days after NMDA injection (4.0 μV) (FIGS. 13B and 13D), reflecting a severe loss of RGC functionality after NMDA-induced excitotoxicity. Significantly, CaMKII reactivation via gene transfer of CaMKIIα T286D preserved PERG responses to levels similar to those recorded in the uninjured retina (FIGS. 13C and 13D).

After leaving the eye, visual information travels through several relay centers of the brain such as LGN and SC, and ultimately reaches the primary visual cortex. Next tested whether preserved RGC responses could be transmitted to the primary visual cortex in vivo. We recorded pattern visually evoked potentials (PVEPs) in primary visual cortices (Porciatti et al., 1999) from the uninjured, NMDA injured, and CaMKIIα T286D treatment groups. Patterned visual stimuli elicited prominent responses (104.8 μV) in the uninjured animals (FIG. 7E), which were markedly reduced (20.9 μV) after NMDA damage (FIG. 13F). Remarkably, CaMKIIα T286D treatment preserved PVEP responses to levels that were comparable to the uninjured condition (FIGS. 13G and 13H). Our results demonstrate that CaMKII reactivation preserves functionality from excitotoxic damages for the entire visual pathway, from the retina to the primary visual cortex in the brain.

To test whether CaMKII-mediated protection of the visual pathway truly preserves vision, we carried out multiple vision-based behavioral tests. First, we used the visual water task to quantitatively measure visual acuity of mice after treatment with CaMKIIα, T286D. The visual water task, a two alternative forced-choice visual behavioral test (Ecker et al., 2010; Prusky et al., 2000), takes advantage of rodents' trained ability to swim to a submerged hidden platform, the location of which is associated with visual stimuli displayed on a computer monitor (FIG. 13I). Mice were trained to swim toward a low spatial frequency (0.05 cycle/degree) grating. Subsequently, the spatial frequency was gradually increased. The visual acuity (i.e., the spatial frequency threshold) is determined when animals make fewer than 70% correct choices. The visual acuity was measured at ˜0.515 c/d in the uninjured mice (FIGS. 13J ad 13M), which dropped to ˜0.128 c/d following NMDA damage (FIGS. 13K and 13M). Importantly, CaMKIIα, T286D treatment significantly improved the acuity to ˜0.388 c/d (FIGS. 13L and 13M).

Then we performed the visual cliff test to assess the maintained ability to discriminate visual depth after CaMKIIα, T286D treatment. This test is based on mice's innate tendency to avoid the deep side and step on to the shallow side of a visual cliff (Fox, 1965; Gu et al., 2018). Mice were placed on the center platform between the deep side and the shallow side of the cliff, and their choices to step towards either side were recorded (FIG. 13N). In the uninjured group, 11 out of 12 mice chose the shallow (safe) side, consistent with previous reports (Fox, 1965; Gu et al., 2018). Significantly worse performance was recorded after NMDA damage, with 7 out of 12 mice choosing the shallow side. By contrast, all 12 CaMKIIα, T286D-treated mice choose the shallow side (FIG. 13O).

Lastly, we evaluated innate defensive responses of mice to looming visual stimuli representing environmental threats. The looming experiment was conducted in an enclosure with a shelter for the mouse to hide, a camera to record the mouse behavior, and an overhead monitor to display looming stimuli (FIG. 13P) (Koehler et al., 2019; Lim et al., 2016; Yilmaz and Meister, 2013). In response to looming stimuli, mice with normal vision consistently displayed one or more of the following behaviors: freezing, fleeing to the shelter, and tail rattling, consistent with previous studies (Koehler et al., 2019; Lim et al., 2016; Salay et al., 2018; Yilmaz and Meister, 2013). As a result, we recorded the mouse as a responder to looming stimuli if it reacted with one or more of these behaviors. In the uninjured group, all 12 mice were responders. After NMDA damage, only 3 out of 12 mice responded to looming stimuli. Remarkably, 11 out of 12 CaMKIIα T286D-treated mice responded to looming stimuli (FIG. 13Q).

Taken together, our results for the first time provide in vivo evidence that CaMKII-mediated RGC protection is capable of preserving functional vision.

Although examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

### REFERENCES

Ahn, S., Olive, M., Aggarwal, S., Krylov, D., Ginty, D. D., and Vinson, C. (1998). A dominant-negative inhibitor of CREB reveals that it is a general mediator of stimulus-dependent transcription of c-fos. Mol Cell Biol 18, 967-977.
Almasieh, M., and Levin, L. A. (2017). Neuroprotection in Glaucoma: Animal Models and Clinical Trials. Annu Rev Vis Sci 3, 91-120.
Ashpole, N. M., and Hudmon, A. (2011). Excitotoxic neuroprotection and vulnerability with CaMKII inhibition. Mol Cell Neurosci 46, 720-730.
Ashpole, N. M., Song, W., Brustovetsky, T., Engleman, E. A., Brustovetsky, N., Cummins, T. R., and Hudmon, A. (2012). Calcium/calmodulin-dependent protein kinase II (CaMKII) inhibition induces neurotoxicity via dysregulation of glutamate/calcium signaling and hyperexcitability. J Biol Chem 287, 8495-8506.
Barco, A., Alarcon, J. M., and Kandel, E. R. (2002). Expression of constitutively active CREB protein facilitates the late phase of long-term potentiation by enhancing synaptic capture. Cell 108, 689-703.
Benowitz, L. I., He, Z., and Goldberg, J. L. (2017). Reaching the brain: Advances in optic nerve regeneration. Exp Neurol 287, 365-373.
Berkelaar, M., Clarke, D. B., Wang, Y. C., Bray, G. M., and Aguayo, A. J. (1994). Axotomy results in delayed death and apoptosis of retinal ganglion cells in adult rats. J Neurosci 14, 4368-4374.
Berridge, M. J., Lipp, P., and Bootman, M. D. (2000). The versatility and universality of calcium signalling. Nat Rev Mol Cell Bio 1, 11-21.
Berry, M., Ahmed, Z., and Logan, A. (2018). Return of function after CNS axon regeneration: Lessons from injury-responsive intrinsically photosensitive and alpha retinal ganglion cells. Prog Retin Eye Res.
Calkins, D. J. (2012). Critical pathogenic events underlying progression of neurodegeneration in glaucoma. Prog Retin Eye Res 31, 702-719.
Chitranshi, N., Dheer, Y., Mirzaei, M., Wu, Y., Salekdeh, G. H., Abbasi, M., Gupta, V., Vander Wall, R., You, Y., Graham, S. L., et al. (2019). Loss of Shp2 Rescues BDNF/TrkB Signaling and Contributes to Improved Retinal Ganglion Cell Neuroprotection. Mol Ther 27, 424-441.
Chou, T. H., Bohorquez, J., Toft-Nielsen, J., Ozdamar, O., and Porciatti, V. (2014). Robust Mouse Pattern Electroretinograms Derived Simultaneously From Each Eye Using a Common Snout Electrode. Invest Ophth Vis Sci 55, 2469-2475.
Coultrap, S. J., Buard, I., Kulbe, J. R., Dell'Acqua, M. L., and Bayer, K. U. (2010). CaMKII autonomy is substrate-dependent and further stimulated by Ca2+/calmodulin. J Biol Chem 285, 17930-17937.
Deisseroth, K., Bito, H., and Tsien, R. W. (1996). Signaling from synapse to nucleus: postsynaptic CREB phosphorylation during multiple forms of hippocampal synaptic plasticity. Neuron 16, 89-101.
Dhande, O. S., Stafford, B. K., Lim, J. H. A., and Huberman, A. D. (2015). Contributions of Retinal Ganglion Cells to Subcortical Visual Processing and Behaviors. Annu Rev Vis Sc 1, 291-328.
Dong, Z., Saikumar, P., Weinberg, J. M., and Venkatachalam, M. A. (2006). Calcium in cell injury and death. Annu Rev Pathol-Mech 1, 405-434.
Ecker, J. L., Dumitrescu, O. N., Wong, K. Y., Alam, N. M., Chen, S. K., LeGates, T., Renna, J. M., Prusky, G. T., Berson, D. M., and Hattar, S. (2010). Melanopsin-expressing retinal ganglion-cell photoreceptors: cellular diversity and role in pattern vision. Neuron 67, 49-60.
Fong, Y. L., Taylor, W. L., Means, A. R., and Soderling, T. R. (1989). Studies of the regulatory mechanism of Ca2+/calmodulin-dependent protein kinase II. Mutation of threonine 286 to alanine and aspartate. J Biol Chem 264, 16759-16763.
Fox, M. W. (1965). The visual cliff test for the study of visual depth perception in the mouse. Anim Behav 13, 232-233.
Goebel, D. J. (2009). Selective blockade of CaMKII-alpha inhibits NMDA-induced caspase-3-dependent cell death but does not arrest PARP-1 activation or loss of plasma membrane selectivity in rat retinal neurons. Brain Res 1256, 190-204.
Gomes, J. R., Costa, J. T., Melo, C. V., Felizzi, F., Monteiro, P., Pinto, M. J., Inacio, A. R., Wieloch, T., Almeida, R. D., Graos, M., et al. (2012). Excitotoxicity downregulates TrkB. FL signaling and upregulates the neuroprotective truncated TrkB receptors in cultured hippocampal and striatal neurons. J Neurosci 32, 4610-4622.
Gu, L., Bok, D., Yu, F., Caprioli, J., and Piri, N. (2018). Downregulation of splicing regulator RBFOX1 compromises visual depth perception. PLoS One 13, e0200417.
Hanson, P. I., Meyer, T., Stryer, L., and Schulman, H. (1994). Dual Role of Calmodulin in Autophosphorylation of Multifunctional Cam Kinase May Underlie Decoding of Calcium Signals. Neuron 12, 943-956.
Harada, T., Harada, C., Nakamura, K., Quah, H. M., Okumura, A., Namekata, K., Saeki, T., Aihara, M., Yoshida, H., Mitani, A., et al. (2007). The potential role of glutamate transporters in the pathogenesis of normal tension glaucoma. J Clin Invest 117, 1763-1770.
Hardingham, G. E., and Bading, H. (2010). Synaptic versus extrasynaptic NMDA receptor signalling: implications for neurodegenerative disorders. Nat Rev Neurosci 11, 682-696.
Hartwick, A. T. E., Hamilton, C. M., and Baldridge, W. H. (2008). Glutamatergic calcium dynamics and deregulation of rat retinal ganglion cells. J Physiol-London 586, 3425-3446.
He, Z., and Jin, Y. (2016). Intrinsic Control of Axon Regeneration. Neuron 90, 437-451.
Herrera, E., and Mason, C. A. (2007). 3.23—The Evolution of Crossed and Uncrossed Retinal Pathways in Mammals. In Evolution of Nervous Systems, J. H. Kaas, ed. (Oxford: Academic Press), pp. 307-317.
Hu, Y., Park, K. K., Yang, L., Wei, X., Yang, Q., Cho, K. S., Thielen, P., Lee, A. H., Cartoni, R., Glimcher, L. H., et al. (2012). Differential effects of unfolded protein response pathways on axon injury-induced death of retinal ganglion cells. Neuron 73, 445-452.
Hudmon, A., and Schulman, H. (2002a). Neuronal Ca2+/calmodulin-dependent protein kinase II: The role of structure and autoregulation in cellular function. Annu Rev Biochem 71, 473-510.
Hudmon, A., and Schulman, H. (2002b). Structure-function of the multifunctional Ca2+/calmodulin-dependent protein kinase II. Biochem J 364, 593-611.
Ito, Y. A., Belforte, N., Cueva Vargas, J. L., and Di Polo, A. (2016). A Magnetic Microbead Occlusion Model to Induce Ocular Hypertension-Dependent Glaucoma in Mice. J Vis Exp, e53731.
Khatib, T. Z., and Martin, K. R. (2017). Protecting retinal ganglion cells. Eye (Lond) 31, 218-224.
Koehler, C. C., Hall, L. M., Hellmer, C. B., and Ichinose, T. (2019). Using Looming Visual Stimuli to Evaluate Mouse Vision. J Vis Exp.
Laabich, A., and Cooper, N. G. (2000). Neuroprotective effect of AIP on N-methyl-D-aspartate-induced cell death in retinal neurons. Brain Res Mol Brain Res 85, 32-40.
Laabich, A., Li, G., and Cooper, N. G. (2000). Calcium/calmodulin-dependent protein kinase II containing a nuclear localizing signal is altered in retinal neurons exposed to N-methyl-D-aspartate. Brain Res Mol Brain Res 76, 253-265.
Lafuente, M. P., Villegas-Perez, M. P., Selles-Navarro, I., Mayor-Torroglosa, S., De Imperial, J. M., and Vidal-Sanz, M. (2002). Retinal ganglion cell death after acute retinal ischemia is an ongoing process whose severity and duration depends on the duration of the insult. Neuroscience 109, 157-168.
Laha, B., Stafford, B. K., and Huberman, A. D. (2017). Regenerating optic pathways from the eye to the brain. Science 356, 1031-1034.
Levin, L. A., and Gordon, L. K. (2002). Retinal ganglion cell disorders: types and treatments. Prog Retin Eye Res 21, 465-484.
Lim, J. H., Stafford, B. K., Nguyen, P. L., Lien, B. V., Wang, C., Zukor, K., He, Z., and Huberman, A. D. (2016). Neural activity promotes long-distance, target-specific regeneration of adult retinal axons. Nat Neurosci 19, 1073-1084.
Lipton, S. A., and Rosenberg, P. A. (1994). Excitatory amino acids as a final common pathway for neurologic disorders. N Engl J Med 330, 613-622.
Lisman, J., Schulman, H., and Cline, H. (2002). The molecular basis of CaMKII function in synaptic and behavioural memory. Nat Rev Neurosci 3, 175-190.
Lonze, B. E., and Ginty, D. D. (2002). Function and regulation of CREB family transcription factors in the nervous system. Neuron 35, 605-623.
Lucas, D. R., and Newhouse, J. P. (1957). The toxic effect of sodium L-glutamate on the inner layers of the retina. AMA Arch Ophthalmol 58, 193-201.
Ma, H., Groth, R. D., Cohen, S. M., Emery, J. F., Li, B., Hoedt, E., Zhang, G., Neubert, T. A., and Tsien, R. W. (2014). gammaCaMKII shuttles Ca(2)(+)/CaM to the nucleus to trigger CREB phosphorylation and gene expression. Cell 159, 281-294.
Malihi, M., Moura Filho, E. R., Hodge, D. O., and Sit, A. J. (2014). Long-term trends in glaucoma-related blindness in Olmsted County, Minnesota. Ophthalmology 121, 134-141.
Manabe, S., and Lipton, S. A. (2003). Divergent NMDA signals leading to proapoptotic and antiapoptotic pathways in the rat retina. Invest Ophth Vis Sci 44, 385-392.
Massey, S. C., and Miller, R. F. (1987). Excitatory amino acid receptors of rod- and cone-driven horizontal cells in the rabbit retina. J Neurophysiol 57, 645-659.
Massey, S. C., and Miller, R. F. (1990). N-methyl-D-aspartate receptors of ganglion cells in rabbit retina. J Neurophysiol 63, 16-30.
Miller, S. G., Patton, B. L., and Kennedy, M. B. (1988). Sequences of Autophosphorylation Sites in Neuronal Type-Ii Cam Kinase That Control Ca-2+-Independent Activity. Neuron 1, 593-604.
Nagahara, A. H., and Tuszynski, M. H. (2011). Potential therapeutic uses of BDNF in neurological and psychiatric disorders. Nat Rev Drug Discov 10, 209-219.
Nickells, R. W., Howell, G. R., Soto, I., and John, S. W. (2012). Under pressure: cellular and molecular responses during glaucoma, a common neurodegeneration with axonopathy. Annu Rev Neurosci 35, 153-179.
Olney, J. W. (1982). The toxic effects of glutamate and related compounds in the retina and the brain. Retina 2, 341-359.
Park, K. K., Liu, K., Hu, Y., Smith, P. D., Wang, C., Cai, B., Xu, B., Connolly, L., Kramvis, I., Sahin, M., et al. (2008). Promoting axon regeneration in the adult CNS by modulation of the PTEN/mTOR pathway. Science 322, 963-966.
Parsons, M. P., and Raymond, L. A. (2014). Extrasynaptic NMDA receptor involvement in central nervous system disorders. Neuron 82, 279-293.
Patton, B. L., Miller, S. G., and Kennedy, M. B. (1990). Activation of type II calcium/calmodulin-dependent protein kinase by Ca2+/calmodulin is inhibited by autophosphorylation of threonine within the calmodulin-binding domain. J Biol Chem 265, 11204-11212.
Pi, H. J., Otmakhov, N., El Gaamouch, F., Lemelin, D., De Koninck, P., and Lisman, J. (2010a). CaMKII control of spine size and synaptic strength: role of phosphorylation states and nonenzymatic action. Proc Natl Acad Sci USA 107, 14437-14442.
Pi, H. J., Otmakhov, N., Lemelin, D., De Koninck, P., and Lisman, J. (2010b). Autonomous CaMKII can promote either long-term potentiation or long-term depression, depending on the state of T305/T306 phosphorylation. J Neurosci 30, 8704-8709.
Porciatti, V. (2007). The mouse pattern electroretinogram. Doc Ophthalmol 115, 145-153.
Porciatti, V., Pizzorusso, T., and Maffei, L. (1999). The visual physiology of the wild type mouse determined with pattern VEPs. Vision Res 39, 3071-3081.
Prilloff, S., Noblejas, M. I., Chedhomme, V., and Sabel, B. A. (2007). Two faces of calcium activation after optic nerve trauma: life or death of retinal ganglion cells in vivo depends on calcium dynamics. Eur J Neurosci 25, 3339-3346.
Prusky, G. T., West, P. W. R., and Douglas, R. M. (2000). Behavioral assessment of visual acuity in mice and rats. Vision Research 40, 2201-2209.
Saggu, S. K., Chotaliya, H. P., Blumbergs, P. C., and Casson, R. J. (2010). Wallerian-like axonal degeneration in the optic nerve after excitotoxic retinal insult: an ultrastructural study. BMC Neurosci 11, 97.
Salay, L. D., Ishiko, N., and Huberman, A. D. (2018). A midline thalamic circuit determines reactions to visual threat. Nature 557, 183-189.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat Methods 9, 676-682.
Schworer, C. M., Colbran, R. J., Keefer, J. R., and Soderling, T. R. (1988). Ca2+/calmodulin-dependent protein kinase II. Identification of a regulatory autophosphorylation site adjacent to the inhibitory and calmodulin-binding domains. J Biol Chem 263, 13486-13489.
Seitz, R., and Tamm, E. R. (2013). N-methyl-D-aspartate (NMDA)-mediated excitotoxic damage: a mouse model of acute retinal ganglion cell damage. Methods Mol Biol 935, 99-109.
Sheng, M., Thompson, M. A., and Greenberg, M. E. (1991). Creb—a Ca2+-Regulated Transcription Factor Phosphorylated by Calmodulin-Dependent Kinases. Science 252, 1427-1430.
Sisk, D. R., and Kuwabara, T. (1985). Histologic changes in the inner retina of albino rats following intravitreal injection of monosodium L-glutamate. Graefes Arch Clin Exp Ophthalmol 223, 250-258.
Sun, F., Park, K. K., Belin, S., Wang, D., Lu, T., Chen, G., Zhang, K., Yeung, C., Feng, G., Yankner, B. A., et al. (2011). Sustained axon regeneration induced by co-deletion of PTEN and SOCS3. Nature 480, 372-375.
Terashima, T., Ochiishi, T., and Yamauchi, T. (1994). Immunocytochemical Localization of Calcium/Calmodulin-Dependent Protein-Kinase-Ii Isoforms in the Ganglion-Cells of the Rat Retina—Immunofluorescence Histochemistry Combined with a Fluorescent Retrograde Tracer. Brain Res 650, 133-139.
Tham, Y. C., Li, X., Wong, T. Y., Quigley, H. A., Aung, T., and Cheng, C. Y. (2014). Global prevalence of glaucoma and projections of glaucoma burden through 2040: a systematic review and meta-analysis. Ophthalmology 121, 2081-2090.
Thiel, G., Czernik, A. J., Gorelick, F., Nairn, A. C., and Greengard, P. (1988). Ca2+/calmodulin-dependent protein kinase II. identification of threonine-286 as the autophosphorylation site in the alpha subunit associated with the generation of Ca2+-independent activity. Proc Natl Acad Sci USA 85, 6337-6341.
Thoreson, W. B., and Witkovsky, P. (1999). Glutamate receptors and circuits in the vertebrate retina. Prog Retin Eye Res 18, 765-810.
Tran, N. M., Shekhar, K., Whitney, I. E., Jacobi, A., Benhar, I., Hong, G., Yan, W., Adiconis, X., Arnold, M. E., Lee, J. M., et al. (2019). Single-Cell Profiles of Retinal Ganglion Cells Differing in Resilience to Injury Reveal Neuroprotective Genes. Neuron 104, 1039-1055 e1012.
Vorwerk, C. K., Lipton, S. A., Zurakowski, D., Hyman, B. T., Sabel, B. A., and Dreyer, E. B. (1996). Chronic low-dose glutamate is toxic to retinal ganglion cells. Toxicity blocked by memantine. Invest Ophthalmol Vis Sci 37, 1618-1624.
Wang, Q., Zhuang, P., Huang, H., Li, L., Liu, L., Webber, H. C., Dalal, R., Siew, L., Fligor, C. M., Chang, K. C., et al. (2020). Mouse gamma-Synuclein Promoter-Mediated Gene Expression and Editing in Mammalian Retinal Ganglion Cells. J Neurosci 40, 3896-3914.
Watkins, T. A., Wang, B., Huntwork-Rodriguez, S., Yang, J., Jiang, Z., Eastham-Anderson, J., Modrusan, Z., Kaminker, J. S., Tessier-Lavigne, M., and Lewcock, J. W. (2013). DLK initiates a transcriptional program that couples apoptotic and regenerative responses to axonal injury. Proc Natl Acad Sci USA 110, 4039-4044.
Weinreb, R. N., Leung, C. K., Crowston, J. G., Medeiros, F. A., Friedman, D. S., Wiggs, J. L., and Martin, K. R. (2016). Primary open-angle glaucoma. Nat Rev Dis Primers 2, 16067.
Welsbie, D. S., Yang, Z., Ge, Y., Mitchell, K. L., Zhou, X., Martin, S. E., Berlinicke, C. A., Hackler, L., Jr., Fuller, J., Fu, J., et al. (2013). Functional genomic screening identifies dual leucine zipper kinase as a key mediator of retinal ganglion cell death. Proc Natl Acad Sci USA 110, 4045-4050.
Williams, P. A., Harder, J. M., Foxworth, N. E., Cochran, K. E., Philip, V. M., Porciatti, V., Smithies, O., and John, S. W. (2017). Vitamin B3 modulates mitochondrial vulnerability and prevents glaucoma in aged mice. Science 355, 756-760.
Yang, L., Li, S., Miao, L., Huang, H., Liang, F., Teng, X., Xu, L., Wang, Q., Xiao, W., Ridder, W. H., 3rd, et al. (2016). Rescue of Glaucomatous Neurodegeneration by Differentially Modulating Neuronal Endoplasmic Reticulum Stress Molecules. J Neurosci 36, 5891-5903.
Yilmaz, M., and Meister, M. (2013). Rapid innate defensive responses of mice to looming visual stimuli. Curr Biol 23, 2011-2015.
Zhang, J., Ackman, J. B., Xu, H. P., and Crair, M. C. (2011). Visual map development depends on the temporal pattern of binocular activity in mice. Nat Neurosci 15, 298-307.
Zhang, J., Cai, C. Y., Wu, H. Y., Zhu, L. J., Luo, C. X., and Zhu, D. Y. (2016). CREB-mediated synaptogenesis and neurogenesis is crucial for the role of 5-HTla receptors in modulating anxiety behaviors. Sci Rep 6, 29551.

What is claimed is:

1. A method of decreasing degeneration of retinal ganglion cells in a subject, comprising
administering to the subject a composition to increase activity of a calcium-calmodulin dependent kinase (CaMK), wherein the composition comprises the CaMK or a polynucleotide encoding the CaMK.

2. The method of claim 1, wherein the composition further comprises a vector.

3. The method of claim 2, wherein the vector comprises a viral vector.

4. The method of claim 3, wherein the vector comprises an adeno-associated vector.

5. The method of any one of claim 1 through 4, wherein the CaMK is selected from one or more of CaMKI, CaMKII, and CaMKIV.

6. The method of any one of claims 1 through 5, wherein the CaMK is selected from one or more of CaMKIIα, CaMKIIβ, CaMKIIγ, and CaMKIIδ.

7. The method of any one of claims 1-6, wherein the CaMK is constitutively active.

8. The method of claim 7, wherein the CaMKII is selected from one or both of a CaMKIIα comprising a T286D substitution and a CaMKIIβ comprising a T287D substitution.

9. The method of any one of claims 1 through 8, wherein the composition comprises a polynucleotide encoding the CaMK.

10. The method of claim 9, wherein the polynucleotide further comprises a retinal ganglion cell promoter.

11. The method of claim 10, wherein the retinal ganglion cell promoter comprises a gamma-Synuclein promoter, or a Synapsin 1 promoter, or a Neurofilament Heavy promoter, or a Thy-I cell surface antigen promoter.

12. The method of claim 10, wherein the retinal ganglion cell promoter comprises a gamma-Synuclein promoter.

13. The method of any one of claims 1, 2, and 5 through 8, wherein the composition comprises the CaMK.

14. The method of any one of claims 1 through 13, wherein the administering comprises intraocular administration.

15. The method of any one of claims 1 through 13, wherein the administering comprises systemic administration.

16. The method of any one of claims 1 through 15, wherein the subject has or is at risk for having one or more of glaucoma, diabetic retinopathy, retinal ischemia, and optic nerve injury.

17. The method of any one of claims 1 through 16, wherein preventing degeneration comprises preventing reduction of one or both of retinal ganglion cell somata and retinal ganglion call axons.

18. A method of treating vision loss in a subject, comprising
administering to the subject a composition to increase activity of a calcium-calmodulin dependent kinase (CaMK), wherein the composition comprises the CaMK or a polynucleotide encoding the CaMK.

19. The method of claim 18, wherein the composition further comprises a vector.

20. The method of claim 19, wherein the vector comprises a viral vector.

21. The method of claim 19, wherein the vector comprises an adeno-associated vector.

22. The method of any one of claim 18 through 21, wherein the CaMK is selected from one or more of CaMKI, CaMKII, and CaMKIV.

23. The method of any one of claims 18 through 22, wherein the CaMK is selected from one or more of CaMKIIα, CaMKIIβ, CaMKIIγ, and CaMKIIδ.

24. The method of any one of claims 18 through 23, wherein the CaMK is constitutively active.

25. The method of claim 24, wherein the CaMKII is selected from one or both of a CaMKIIα comprising a T286D substitution and a CaMKIIβ comprising a T287D substitution.

26. The method of any one of claims 18 through 25, wherein the composition comprises a polynucleotide encoding the CaMK.

27. The method of claim 26, wherein the polynucleotide further comprises a retinal ganglion cell promoter.

28. The method of claim 27, wherein the retinal ganglion cell promoter comprises a gamma-Synuclein promoter, or a Synapsin 1 promoter, or a Neurofilament Heavy promoter, or a Thy-1 cell surface antigen promoter.

29. The method of claim 27, wherein the retinal ganglion cell promoter comprises a gamma-Synuclein promoter.

30. The method of any one of claims 18, 19, and 22 through 25, wherein the composition comprises the CaMK.

31. The method of any one of claims 18 through 30, wherein the administering comprises intraocular administration.

32. The method of any one of claims 18 through 30, wherein the administering comprises systemic administration.

33. The method of any one of claims 18 through 32, wherein the subject has or is at risk for having one or more of glaucoma, diabetic retinopathy, retinal ischemia, and optic nerve injury.

34. The method of any one of claims 18 through 32, wherein treating comprises preventing reduction of one or both of retinal ganglion cell somata and retinal ganglion call axons.

35. The method of any one of claims 18 through 34, wherein treating comprises preventing vision loss.

36. A pharmaceutical composition, comprising
a polynucleotide and a vector, wherein the polynucleotide comprises a retinal ganglion cell promoter and encodes a calcium-calmodulin dependent kinase (CaMK).

37. The pharmaceutical composition of claim 36, wherein the retinal ganglion cell promoter comprises a gamma-Synuclein promoter, or a Synapsin 1 promoter, or a Neurofilament Heavy promoter, or a Thy-1 cell surface antigen promoter.

38. The pharmaceutical composition of claim 36, wherein the retinal ganglion cell promoter comprises a gamma-Synuclein promoter.

39. The pharmaceutical composition of any one of claims 36 through 38, wherein the vector comprises a viral vector.

40. The pharmaceutical composition of claim 39, wherein the vector comprises an adeno-associated vector.

41. The pharmaceutical composition of any one of claim 36 through 40, wherein the CaMK is selected from one or more of CaMKI, CaMKII, and CaMKIV.

42. The pharmaceutical composition of any one of claim 36 through 41, wherein the CaMK is selected from one or more of CaMKIIα, CaMKIIβ, CaMKIIγ, and CaMKIIδ.

43. The pharmaceutical composition of any one of claims 36 through 42, wherein the CaMK is constitutively active.

44. The pharmaceutical composition of claim 43, wherein the CaMKII is selected from one or both of a CaMKIIα comprising a T286D substitution and a CaMKIIβ comprising a T287D substitution.

45. A method of decreasing degeneration of retinal ganglion cells in a subject, comprising
administering to the subject a composition to increase activity of a cyclic-AMP response element-binding protein (CREB) activity, wherein the composition comprises the CREB or a polynucleotide encoding the CREB.

46. The method of claim 45, wherein the composition further comprises a vector.

47. The method of claim 46, wherein the vector comprises a viral vector.

48. The method of claim 47, wherein the vector comprises an adeno-associated vector.

49. The method of any one of claim 45 through 48, wherein the CREB is constitutively active.

50. The method of claim 49, wherein the CREB comprises VP-16 CREB.

51. The method of any one of claims 45 through 50, wherein the composition comprises a polynucleotide encoding the CREB.

52. The method of claim 51, wherein the polynucleotide further comprises a retinal ganglion cell promoter.

53. The method of claim 52, wherein the retinal ganglion cell promoter comprises a gamma-Synuclein promoter, or a Synapsin 1 promoter, or a Neurofilament Heavy promoter, or a Thy-I cell surface antigen promoter.

54. The method of claim 52, wherein the retinal ganglion cell promoter comprises a gamma-Synuclein promoter.

55. The method of any one of claims 45, 46, 49, and 50, wherein the composition comprises the CREB.

56. The method of any one of claims 45 through 55, wherein the administering comprises intraocular administration.

57. The method of any one of claims 45 through 55, wherein the administering comprises systemic administration.

58. The method of any one of claims 45 through 57, wherein the subject has or is at risk for having one or more of glaucoma, diabetic retinopathy, retinal ischemia, and optic nerve injury.

59. The method of any one of claims 45 through 58, wherein preventing degeneration comprises preventing reduction of one or both of retinal ganglion cell somata and retinal ganglion call axons.

60. A method of treating vision loss in a subject, comprising administering to the subject a composition to increase activity of a cyclic-AMP response element-binding protein (CREB) activity, wherein the composition comprises the CREB or a polynucleotide encoding the CREB.

61. The method of claim 60, wherein the composition further comprises a vector.

62. The method of claim 61, wherein the vector comprises a viral vector.

63. The method of claim 61, wherein the vector comprises an adeno-associated vector.

64. The method of any one of claims 60 through 63, wherein the CREB is constitutively active.

65. The method of claim 64, wherein the CREB comprises VP-16 CREB.

66. The method of any one of claims 60 through 65, wherein the composition comprises a polynucleotide encoding the CREB.

67. The method of claim 66, wherein the polynucleotide further comprises a retinal ganglion cell promoter.

68. The method of claim 67, wherein the retinal ganglion cell promoter comprises a gamma-Synuclein promoter, or a Synapsin 1 promoter, or a Neurofilament Heavy promoter, or a Thy-1 cell surface antigen promoter.

69. The method of claim 67, wherein the retinal ganglion cell promoter comprises a gamma-Synuclein promoter.

70. The method of any one of claims 60, 61, 64, and 65, wherein the composition comprises the CREB.

71. The method of any one of claims 60 through 70, wherein the administering comprises intraocular administration.

72. The method of any one of claims 60 through 70, wherein the administering comprises systemic administration.

73. The method of any one of claims 60 through 72, wherein the subject has or is at risk for having one or more of glaucoma, diabetic retinopathy, retinal ischemia, and optic nerve injury.

74. The method of any one of claims 60 through 73, wherein treating comprises preventing reduction of one or both of retinal ganglion cell somata and retinal ganglion call axons.

75. The method of any one of claims 60 through 74, wherein treating comprises preventing vision loss.

76. A pharmaceutical composition, comprising
a polynucleotide and a vector, wherein the polynucleotide comprises a retinal ganglion cell promoter and encodes a cyclic-AMP response element-binding protein (CREB).

77. The method of claim 76, wherein the retinal ganglion cell promoter comprises a gamma-Synuclein promoter, or a Synapsin 1 promoter, or a Neurofilament Heavy promoter, or a Thy-1 cell surface antigen promoter.

78. The method of claim 76, wherein the retinal ganglion cell promoter comprises a gamma-Synuclein promoter.

79. The pharmaceutical composition of any one of claims 76 through 78, wherein the vector comprises a viral vector.

80. The pharmaceutical composition of claim 79, wherein the vector comprises an adeno-associated vector.

81. The pharmaceutical composition of any one of claims 76 through 80, wherein the CREB is constitutively active.

82. The pharmaceutical composition of claim 81, wherein the CREB comprises VP-16 CREB.
